(19) United States
(12) Patent Application Publication
(10) Pub. No.: US 2009/0192199 A1
(43) Pub. Date: Jul. 30, 2009

(54) PHARMACOLOGICAL MODULATION OF POSITIVE AMPA RECEPTOR MODULATOR EFFECTS ON NEUROTROPHIN EXPRESSION

(75) Inventors: Julie C. Lauterborn, Huntington Beach, CA (US); Christine M. Gall, Irvine, CA (US); Gary Lynch, Irvine, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: The Regents of the University of California, Oakland, CA (US)

(21) Appl. No.: 12/297,616

(22) PCT Filed: Apr. 19, 2007
(86) PCT No.: PCT/US2007/066947

§ 371 Date: Jan. 26, 2009

Related U.S. Application Data
(60) Provisional application No. 60/793,966, filed on Apr. 20, 2006.

(51) Int. Cl. A61K 31/4439; A61K 31/42; A61K 31/435; A61K 31/4164; A61P 25/28

(52) U.S. Cl. 514/342; 514/380; 514/277; 514/399

(57) ABSTRACT

Antagonists of group 1 metabotropic glutamate receptors (mGluR) potentiate the effect of positive AMPA receptor modulators on neurotrophin expression, such as brain-derived neurotrophic factor (BDNF). The findings described herein suggest a combinatorial approach for drug therapies, using both positive AMPA receptor modulators and mGluR antagonists. to enhance brain neurotrophism.

### CROSS-REFERENCES TO RELATED APPLICATIONS

This application claims benefit of U.S. provisional application Ser. No. 60/793,966, filed Apr. 20, 2006, the disclosure of which is incorporated herein in its entirety by reference.

### STATEMENT AS TO RIGHTS TO INVENTIONS MADE UNDER FEDERALLY SPONSORED RESEARCH AND DEVELOPMENT

This invention was made with Government support under Grant No. NS45260, awarded by the NIH. The Government has certain rights in this invention.

### FIELD OF THE INVENTION

The present invention relates generally to compositions and methods useful for the modulation of mammalian neurotrophic factor expression.

### BACKGROUND OF THE INVENTION

Release of glutamate (Glu), the most abundant excitatory neurotransmitter, at synapses at many sites in the mammalian brain stimulates two classes of postsynaptic glutamate receptors: ionotropic receptors that form membrane ion channels and metabotropic receptors coupled to G proteins. Glu activation of the ionotropic receptors constitutes a base for all brain functions. Ionotropic receptors include the β-amino-3-hydroxy-5-methyl-isoxazole-4-propionic acid (AMPA), or AMPA/quisqualate, receptors, N-methyl-D-aspartic acid (NMDA) receptors and kainite receptors. The first of these mediates a voltage independent fast excitatory post-synaptic current (the fast EPSC) while the NMDA receptor generates a voltage dependent, slow excitatory current. Studies carried out in slices of hippocampus or cortex indicate that the AMPA receptor-mediated fast EPSC is by far the dominant component at most glutaminergic synapses under most circumstances. AMPA receptors are not evenly distributed across the brain but instead are largely restricted to telencephalon (cortex, limbic system, striatum; about 90% of human brain) and cerebellum (Gold et al., 1996, J Comp Neurol 365:541-555). They are found in high concentrations in the superficial layers of neocortex, in each of the major synaptic zones of hippocampus, and in the striatal complex (see, for example, Monaghan et al., 1984, Brain Research 324:160-164; Monyer et al., 1991, Neuron 6:799-810; Geiger et al., 1995, Neuron 15:193-204). Studies in animals and humans indicate that these structures organize complex perceptual-motor processes and provide the substrates for higher-order behaviors. Thus, AMPA receptors mediate transmission in those brain networks responsible for a host of cognitive activities. Further, there is experimental data to suggest that drugs enhancing these receptor currents facilitate communication in brain networks responsible for perceptual-motor integration and higher order behaviors by inducing expression of neurotrophin genes (Lauterborn et al., 2000, J Neurosci 20(1):8-21).

Neurotrophic factors include a number of families of endogenous substances that protect neurons from a variety of pathogenic conditions, support the survival and, in some instances, the growth and biosynthetic activities of neurons (Lindvall et al., 1994, Trends Neurosci 17:490-496; Mattson and Scheff, 1994, J Neurotrauma 11:3-33). A tremendous interest in neurotrophic factors has developed in the hope that they might be used to protect against the neurodegenerative effects of disease (e.g., Parkinson's disease. amyotrophic lateral sclerosis, Alzheimer's disease), normal aging, and physical trauma to the brain (See, e.g., Barinaga et al., 1994, Science 264:772-774; Eide et al., 1993, Exp Neurol 121:200-214).

Given the beneficial function of neurotrophins, there is considerable therapeutic interest in finding novel means to increase their availability in the brain, particularly in a brain of a mammal afflicted with a pathology. The therapeutic use of neurotrophic factors has centered around (i) infusion of exogenous factors into the brain (Fischer et al., 1987, Nature 329(6134):65-68), (ii) implantation of cells genetically engineered to secrete factors into the brain (Gage et al., 1991, Trends Neurosci 14:328-333); Stromberg et: al., 1990, J Neurosci Res 25:405-411), and (iii) the design of techniques for the transport of peripherally applied trophic activities across the blood brain barrier and into the brain (normally the blood brain barrier prevents penetration). A significant disadvantage of these methods is the requirement for invasive procedures or the use of direct neurotransmitter agonists which readily induce seizures and/or disrupt normal neuronal function. There have been fewer efforts designed to identify peripheral agents that can increase endogenous expression in the brain (Carswell, 1993, Exp Neurol 123:36-423; Saporito et al., 1993, Exp Neurol 123:295-302).

One member of the neurotrophin family of factors is brain-derived neurotrophic factor (BDNF). BDNF has been shown to be neuroprotective, to support neuronal survival and to have positive effects on the physiological and morphological properties of neurons. The loss, or abnormally low expression, of this protein appears to contribute to depression, anxiety, and cognitive deficits.

Positive AMPA receptor modulators, that potentiate AMPA-class glutamate receptor mediated currents, have been demonstrated to increase BDNF expression (i.e., gene transcription and protein synthesis) by hippocampal and neocortical neurons indicating that these drugs may be useful therapeutics for enhancing neurotrophin expression and, secondary to this, supporting neuronal viability and function (Lauterborn et al., 2000, J Neurosci 20:8-21; Legutko et al., 2001, Neuropharmacology 40:1019-27; Mackowiak et al., 2002, Neuropharmacology 43:1-10; Lauterborn et al., 2003, J Pharmacol Exp Ther 307, 297-305). The mechanism by which this occurs involves activation of L-type voltage sensitive calcium channels leading to increases in intracellular calcium. Increases in calcium, in turn, activate subcellular signaling to eventually increase BDNF gene transcription (Ghosh et al., 1994, Science 263:1618-23; Tao et al., 1998, Neuron 20:709-26; Lauterborn et al., 2000, J Neurosci 20:8-21).

The list of compounds that modulate AMPA-type glutamate receptors includes, for example the nootropic drug aniracetam (Ito et al., 1990, J Physiol 424:533-543), diazoxide and cyclothiazide (CTZ), two benzothiadiazides used clinically as antihypertensives or diuretics (Yamada and Rotham, 1992, J Physiol (LOnd) 458:409-423; Yamada and Tang, 1993, J Neurosci 13:3904-3915).

Positive AMPA receptor modulators also include a relatively new and still evolving class of compounds called AMPAKINE® drugs, a group of small benzamide (benzoylpiperidine) compounds that were originally derived from aniracetam (Arai et al., 2000, Mol Pharmacol 58(4):802-13). AMPAKINES® slow AMPA-type glutamate receptor deactivation (channel closing, transmitter dissociation) and desensitization rates and thereby enhance fast excitatory synaptic currents in vitro and in vivo and AMPA receptor currents in excised patches (Arai et al., 1994, Brain Res 638:343-346; Staubli et al., 1994, Proc Natl Acad Sci USA 91:777-781; Arai et al., 1996, J Pharmacol Exp Ther 278:627-638; Arai et al., 2000, Mol Pharmacol 58(4):802-813). The drugs do not have agonistics or antagonistic properties but rather modulate the receptor rate constants for transmitter binding, channel opening and desensitization (Arai et al., 1996, J Pharmacol Exp Ther 278:627-638).

AMPAKINES® are of particular interest with regard to neurotrophin regulation because they cross the blood-brain barrier (Staubli et al., 1994, Proc Natl Acad Sci USA 91:11158-11162).

AMPAKINES® have been shown to improve memory encoding in rats and possibly humans across a variety of experimental paradigms without detectably affecting performance or mood (Staubli et al., 1994, Proc Natl Acad Sci USA 91:777-78; Rogan et al., J Neurosci 17:5928-5935; Ingvar et al., 1997, Exp Neurol 146:553-559; Hampson et al., 1998, J Neurosci 18:2740-2747). Further, it has been reported that AMPAKINES®, though differing in their effects on AMPA-receptor-mediated responses, have similar effects at the behavioral level (Davis et al., 1997, Psychopharmacology (Berl) 133(2):161-7). Moreover, repeated administration of AMPAKINES® produced lasting improvements in learned behaviors without causing evident side effects (Hampson et al., 1998, J Neurosci 18:2748-2763).

CX614 (2H,3H,6aH-pyrrolidino[2″,1″-3′,2′]1,3-oxazino[6′,5′-5,4]benzo[e]1,4-dioxan-10-one; LiD37 or BDP-37) (Arai et al., 1997, Soc Neurosci Abstr 23:313; Hennegrif et al., 1997, J Neurchem 68:2424-2434; Kessler et al., 1998, Brain Res 783:121-126) is an AMPAKINE® that belongs to a benzoxazine subgroup characterized by greater structural rigidity and higher potency. This well-studied AMPAKINE® markedly and reversibly increased brain-derived neurotrophic factor (BDNF) mRNA and protein levels in cultured rat entorhinal/hippocampal slices in a dose-dependent manner over a range in which the drug increased synchronous neuronal discharges (Lauterborn et al., 2000, J Neurosci 20(1):8-21).

The structurally distinct AMPAKINE® CX546 (GR87 or BDP-17) (Rogers et al., 1988, Neurobiol Aging 9:339-349; Hoist et al., 1998, Proc Natl Acad Sci USA 95:2597-2602) gave comparable results (Lauterborn et al, 2000, J Neurosci 20(1):8-21). Further, AMPAKINE®-induced upregulation of BDNF expression was broadly suppressed by AMPA receptor antagonists, but not by NMDA receptor antagonists (Lauterborn et al., 2000, J Neurosci 20(1):8-21). While prolonged infusions of suprathreshold AMPAKINE® concentrations produced peak BDNF mRNA levels at 12 hrs and a return to baseline levels by 48 hr, BDNF protein remained elevated throughout a 48 hrs incubation with the drug (Lauterborn et al., 2000, J Neurosci 20:8-21; Lauterborn et al., 2003, J Pharmacol Exp Ther 307:297-305).

Metabotropic glutamate receptors (mGluR) are G-protein-coupled receptors that include eight subtypes and are classified into three groups according to their sequence homology, biochemical, electrophysiological and pharmacological properties (Pin and Duvoisin, 1995, Neuropharmacology 34:1-26). Receptors belonging to group 1 (mGluR1 and mGluR5) are positively linked to phospholipase C, while group II (mGluR2, mGluR3) and III (mGluR4, mGluR6, mGluR7 and mGluR8) receptors are negatively coupled to adenyl cyclase (Bordi and Ugolini, 1999, Prog Neurobiol 59:55-79). Group I mGluRs work as stimulators of Glu transmission and activate second messenger systems (Conn and Pin, 1997, Annu Rev Pharmacol Toxicol 37:205-237; Knopfel et al., 1997, J Med Chem 38:1417-1424). In particular, activation of group 1 mGluRs stimulates polyphosphoinositide hydrolysis into inositol-1,4,5-triphosphate and diacylglycerol, with ensuing release of intracellular calcium and activation of protein kinase C. While stimulation of mGluR1 resulted in a single peak of intracellular Ca²⁺ level, activation of mGluR5 produces long-term Ca²⁺ oscillations (Nakanishi et al., 1998, Brain Res Brain Res Rev 26:230-235).

Recently, mGluR5 was also implicated in mediating the reinforcing and incentive motivational properties of nicotine, cocaine and food (Paterson and Markou, 2005, Psychopharmacology (Berl) 179(1):255-61), in morphine withdrawal (Rasmussen et al., 2005, Neuropharmacology 48(2): 173-80), in modulating both the maintenance of operant ethanol self-administration and abstinence-induced increases in ethanol intake (Schroeder et al., 2005, Psychopharmacology (Berl) 179(1):262-70) and in regulation of hormone secretion in the endocrine pancreas (Brice et al., 2002, Diabetologia 45(2):242-52; Storto et al., 2006, Mol Pharmacol January 19).

Stimulation of group 1 mGluRs has been shown to facilitate Glu excitatory effects, while their blockade leads to an inhibitory action in the brain (Bruno et al., 1995, Neuropharmacology 34:1089-1098; Conn and Pin, 1997, Annu Rev Pharmacol Toxicol 37:205-237; McDonald et al., 1993, J Neurosci 13:4445-4455). In addition, group 1 mGluR agonists also have been reported to negatively regulate voltage sensitive calcium channels (Choi and Lovinger, 1996, J Neurosci 16:36-45; Sayer 1998, J Neurophysiol 80:1981-8; Lu and Rubel, 2005, J Neurophysiol 93:1418-28).

Antagonists of group 1 mGluRs, such as 2-methyl-6-(phenylethynyi)pyridine (MPEP) and (E)-2-methyl-2-styrylpyridine (SIB 1893), which are specific for mGluR5, are reported to be neuroprotective (Gasparini et al., 1999, Neuropharmacology 38:1493-1503; Chapman et al., 2000, Neuropharmacology 39:1567-1574; Barton et al., 2003, Epilepsy Res 56:17-26). Recently, MPEP was shown to have anxiolytic-like effects involving neuropeptide Y but not GABA_{A} signaling (Pilc et al., 1998, Eur J Pharmacol 349:83-87; Wiero\dot{n}ska et al., 2004, Neuropsychopharmacology 29:514-521; Ballard et al., 2005, Psychopharmacology (Berl) 179(1):218-29).

Recent studies have indicated that mGluR5 can modulate NMDA receptor function in vivo. For example, MPEP can potentiate PCP (phencyclidine)-evoked hyperactivity and PCP-induced disruptions in prepulse inhibition in rats (Henry et al., 2002, Neuropharmacology 43(8):1199-209). Campbell et al. provided further support for mGluR5 modulating NMDA receptor function by showing that MPEP had no effect when administered alone, however, potentiated the disruptions in learning induced by a low dose of PCP and potentiated the impairments in memory induced by PCP (Campbell et al., 2004, Psychopharmacology 173(3):310-8).

More recently, Turle-Lorenzo et al. investigated the effects of MPEP and NMDA receptors and in particular the synergistic effects of L-DOPA and MPEP on the akinetic syndrome observed in bilateral 6-OHDA (6-hydroxydopamine)-lesioned rats (a classical model of Parkinson's disease). They found that L-DOPA had a potent anti-akinetic effect in 6-OHDA-lesioned rats, but this effect was not potentiated by MPEP (Turle-Lorenzo et al., 2005, Psychopharmacology (Berl) 179(1):117-27). Similar results were described by Domenici et al. who reported that MPEP did not potentiate L-DOPA-induced turning in the 6-OHDA model (Dominici et al., 2005, J Neurosci Res 80(5):646-54). In another study, MPEP was shown to not affect episodes of spike- and wave rhythm elicited by low doses of pentetrazol in a rat epileptic seizure model (Lojkova and Mares, 2005, Neuropharmacology 49 Suppl 1:219-29).

Rather, the mGluR selective antagonist MPEP was shown to have a blocking effect, via effects on mGluR5, on the function of another receptor, mGluR1. Bonsi et al. reported that the group 1 non-selective agonist 3,5-DHPG induced a membrane depolarization/inward current and that this effect was prevented by co-application of MPEP (Bonsi et al., 2005, Neuropharmacology 49 Suppl 1:104-113).

Heteromeric receptor complexes comprising adenosine A2A and mGluR5 in striatum have suggested the possibility of synergistic interactions between striatal A2A and mGluR5. Kachron et al., described that locomotion acutely stimulated by MPEP was potentiated by the A2A antagonist KW-6002, both in normal and in dopamine-depleted mice (Kachroo et al., 2005, J Neurosci 25(45):10414-9).

Recently, some synergistic interactions between AMPAKINES® and antipsychiatric drugs were reported with respect to decreased methamphetamine-induced hyperactivity in rats. Interactions between the AMPAKINE® CX516 and low doses of different antipsychiatrics were generally additive and often synergistic (Johnson et al., 1999, J Pharmacol Exp Ther 289(1):392-7). In these studies the AMPAKINE® potentiated the effect of the antipsychiatric drug.

However, to the best knowledge of the applicants, group 1 mGluR5 antagonists, such as MPEP, have not been tested in combination with a positive AMPA receptor modulator, nor has MPEP or any other group 1 mGluR5 antagonist been shown to work in synergism with positive AMPA receptor modulators to further increase expression of a neurotrophic factor, such as BDNF. Nor does the current art suggest a beneficial effect of administering a positive AMPA receptor modulator and a group 1 mGluR5 antagonist in a method for increasing the level of BDNF, for treatment of a pathology characterized by an aberrant expression of a neurotrophic factor, such as BDNF, for improving a cognitive function, for treatment of a psychiatric disorder, for treatment of Fragile X syndrome, for treatment of a sexual dysfunction, or for treatment of a pathology associated with reduced expression of a growth hormone.

Heretofore, there has been no known connection between the effect of a group I mGluR5 antagonist and stimulators of AMPA receptors in the aforementioned methods.

Quite surprisingly, applicants describe studies that show that group 1 mGluR5 antagonist, such as MPEP, potentiate the effect of positive AMPA receptor modulators, such as CX614, on neurotrophin expression, and in particular expression of BDNF. Thus, the modulation of AMPA receptors described herein using both a positive AMPA receptor modulator and a group 1 mGluR5 antagonist represents a novel approach for the treatment of neurological and neuropsychiatric disorders.

### BRIEF SUMMARY OF THE INVENTION

This application discloses the surprising finding that antagonists of the group 1 metabotropic glutamate receptor subtype 5 (mGluR5) potentiate the effects of positive AMPA receptor modulators on BDNF expression in neurons with co-treatment. This is the first demonstration that antagonism of mGluR5 has an effect on activity-dependent BDNF expression.

The findings disclosed herein demonstrate that group 1 mGluR5 antagonists facilitate the effect of positive AMPA receptor modulators on neurotrophin expression, in particular BDNF, and thereby potentiate AMPA receptor modulator effects on BDNF expression. The use of the combined drug treatment (i.e., positive AMPA receptor modulator and group 1 mGluR5 antagonist) lead to greater elevations in BDNF expression than are seen following treatment with the positive AMPA receptor modulator alone. Thus, this invention is particularly useful as a therapeutic treatment where large increases of BDNF may be desired. Greater elevations in BDNF would be expected to be beneficial to synaptic plasticity and to play a role in the reversal of cognitive deficits particularly seen with mental retardation, as well as reduce depression and anxiety. Greater increase in BDNF expression may also lead to greater neuroprotection, neuronal survival and health than can be achieved by treatment with a positive AMPA receptor modulator alone. Thus, generally, methods of the present invention are useful where an increase in neurotrophic factor expression, and in particular an increase in BDNF expression, is desired.

Thus, in one aspect, the present invention provides a method for increasing the level of a neurotrophic factor in a brain of a mammal afflicted with a neurodegenerative pathology. In a preferred embodiment, of the present invention, this method comprises the steps of (a) administering to the mammal an amount of an AMPA-receptor allosteric upmodulator effective to increase the expression of the neurotrophic factor in the brain of the mammal; and (b) administering to the mammal an amount of a group 1 metabotropic glutamate receptor antagonist effective to increase the expression of the neurotrophic factor in the brain of the mammal above the level exhibited by step (a) alone. In one embodiment, the level of the neurotrophic factor is increased at least 25% above the level exhibited by step (a) alone.

Methods and compositions of the present invention are useful to improve a neurodegenerative pathology. In a preferred embodiment, the neurodegenerative pathology is selected from the group consisting of Parkinson's Disease, amyotrophic lateral sclerosis (ALS), Huntington's disease, and Down's Syndrome. In another embodiment, the neurodegenerative pathology is characterized by reduced cognitive activity. In yet another embodiment, the neurodegenerative pathology is a psychiatric disorder. In another preferred embodiment, the neurodegenerative pathology is Fragile X syndrome. The neurodegenerative pathology may also be a sexual dysfunction or characterized by reduced expression of a growth hormone.

In a preferred embodiment, the mammal afflicted with a neurodegenerative pathology is a human.

Methods of the invention are useful to increase the level of a neurotrophic factor in the brain of a mammal afflicted with a neurodegenerative pathology. In one embodiment of the present invention, the neurotrophic factor is selected from the group consisting of brain derived neurotrophic factor, nerve growth factor, glial cell line derived neurotrophic factor, ciliary neurotrophic factor, fibroblast growth factor, and insulin-like growth factor. A preferred neurotrophic factor is brain derived neurotrophic factor.

Preferred are AMPA-receptor allosteric upmodulators and group 1 metabotropic glutamate receptor antagonists that are blood-brain barrier permeant.

Methods and compositions of the present invention comprise various group 1 metabotropic glutanate receptor antagonists. In one embodiment of the present invention, the group 1 metabotropic glutamate receptor antagonist is selected from the group consisting of 2-methyl-6-(phenylethynyl)pyridine (MPEP), 3-[(2-methyl-1,3-thiazol-4-yl)ethynyl]pyridine (MTEP), (E)-2-methyl-6-styryl-pyridine (SIB 1893), N-(3-chlorophenyl)-N′-(4,5-dihyfro-1-methyl-4-oxo-1H-imidazole-2-yl)urea (fenobam), and structural analogs thereof. A preferred group 1 metabotropic glutamate receptor antagonist is MPEP. Another preferred group 1 metabotropic glutamate receptor antagonist is fenobam.

Methods and compositions of the present invention comprise various AMPA-receptor allosteric upmodulators. In one embodiment of the present invention, the AMPA-receptor allosteric upmodulator is selected from the group consisting of CX516, CX546, CX614, CX691, CX929, and structural analogs thereof. A preferred AMPA-receptor allosteric upmodulator is CX614. Another preferred AMPA-receptor allosteric upmodulator is CX516.

In another preferred embodiment of the present invention, the AMPA-receptor allosteric upmodulator is selected from the group consisting of 1, compound 2, compound 3, compound 4, compound 5, compound 6, compound 7, compound 8, compound 9, compound 10, compound II, compound 12, compound 13, compound 14, compound 15, compound 16, compound 17, compound 18, compound 19, compound 20, compound 21, compound 22, compound 23, compound 24, compound 25, compound 26, compound 27, compound 28, compound 29, compound 30, compound 31, compound 32, compound 33, compound 34, compound 35 compound 36, compound 37, compound 38, compound 39, compound 40, compound 41, compound 42, compound 43, compound 44, compound 45 compound 46, compound 47, compound 48, compound 49, compound 50, compound 51, compound 52, compound 53, compound 54, and structural analogs thereof.

In another aspect, the present invention provides a method for increasing in a brain of a mammal afflicted with a neurodegenerative pathology the level of a neurotrophic factor above the level of neurotrophic factor induced by an AMPA-receptor allosteric upmodulator.

In a preferred embodiment, this method comprises the step of administering to the mammal an amount of a group 1 metabotropic glutamate receptor antagonist effective to increase the level of the neurotrophic factor in the brain of the mammal.

This invention also provides pharmaceutical compositions comprising (i) an AMPA-receptor allosteric upmodulator, (ii) a group 1 metabotropic glutamate receptor antagonist, and (iii) a pharmaceutically acceptable carrier.

Further, this invention provides the use of (i) an AMPA-receptor allosteric upmodulator, and (ii) a group 1 metabotropic glutamate receptor antagonist in the manufacture of a medicament. The medicament can be used for increasing in a brain of a mammal afflicted with a neurodegenerative pathology the level of a neurotrophic factor.

In another aspect, the present invention provides kits useful for practicing a method of the present invention. In a preferred embodiment, a kit comprises (i) a first container containing an AMPA-receptor allosteric upmodulator, (ii) a second container containing a group 1 metabotropic glutamate receptor 5 antagonist, and (iii) an instruction for using the AMPA-receptor allosteric upmodulator and the group 1 metabotropic glutamate receptor 5 antagonist for increasing the level of a neurotrophic factor above the level of neurotrophic factor induced by the AMPA-receptor allosteric upmodulator alone.

### BRIEF DESCRIPTION OF THE DRAWINGS

FIG. 1 is a diagram showing that stimulation of group 1 mGluRs leads to internalization of AMPA receptors. Antagonists block this effect. Stimulation of group I mGluRs also leads to (i) activation of protein kinase C (PKC) and release of intracellular calcium stores ([Ca²⁺]) that contributes to down-stream signaling (indicated by dashed lines) and effects on gene expression, and (ii) local protein synthesis in dendritic spines. Glu, glutamine; NMDAR, N-methyl-D-aspartic acid (NMDA) receptor; AMPAR, α-amino-3-hydroxy-5-methyl-4-isoxazolepropionic acid (AMPA) receptor, mGluR, metabotropic glutamate receptor.

FIG. 2 shows that AMPAKINES® increase hippocampal BDNF mRNA expression in vitro. A supra-threshold CX614 dose elevates levels through 24 h. The dark-field photomicrographs show in situ hybridization to BDNF mRNA in sections from control hippocampal organotypic cultures and cultures chronically treated with the AMPAKINE® CX614 for 6-24 hours. As shown, BDNF mRNA levels are markedly elevated by 6 h and begin to decline by 24 h of continuous treatment.

FIG. 3 shows that treatment with GluR5 antagonist MPEP potentiates CX614-induced increases in hippocampal BDNF mRNA. A. BDNF in situ hybridization. B. Quantification of in situ hybridization. Cultured rat hippocampal slices were treated for 3 h with CX614 (50 μM) with or without the group 1 mGluR antagonist MPEP (50 μM) present. In hippocampal stratum granulosum (sg), analysis of BDNF mRNA levels revealed a 6.5-fold increase in cultures treated with the CX614 alone (p<0.001 vs control group). Co-treatment with CX614+MPEP increased BDNF mRNA levels 10.5-fold above control levels (p<0.001), and levels were significantly greater than in CX614 alone group (p<0.01). In CA1 stratum pyramidale, CX614 alone lead to a small but non-significant increased in BDNF mRNA levels. However, co-treatment with CX614+MPEP resulted in a marked increase in expression (p<0.01 vs control group). Treatment with MPEP alone had no effect in any field.

FIG. 4 shows that the effect of CX614 on BDNF expression is dose-dependent. Bar graphs show the effect of a 3 h treatment with various concentrations of CX614 on BDNF cRNA labeling in the dentate gyrus stratum granulosum (SG), CA3 stratum pyramidale (CA3), and CA1 stratum pyramidale (CA1). Graphs show mean density values for each subfield (±SEM; left y-axis applies to SG and right y-axis applies to CA3 and CA1). For the granule cells, a modest increase was seen with 10 μM CX614, and more dramatic increases were seen at higher doses. For the pyramidal cells, only 50 μM CX614 elicited significant increases with 3 h treatment.

FIG. 5 shows that a treatment with a low dose of CX614 is potentiated by mGluR5 antagonist. A. BDNF in situ hybridization. B. Quantification of in situ hybridization. Cultured rat hippocampal slices were treated for 24 h with CX614 (20 μM) with or without the group 1 mGluR5 antagonist MPEP (50 μM) present and analyzed for changes in BDNF expression. In stratum granulosum, there were slightly greater mRNA levels in the CX614+MPEP group than in the CX614 alone group (p<0.05, p<0.01 vs control group). In CA1 stratum pyramidale, 24 h treatment with CX614 alone lead to a small but non-significant increase in BDNF mRNA content. In cultures co-treated with CX614+MPEP, BDNF mRNA levels in CA1 were markedly increased above control levels (p<0.01) and greater than in the CX614 alone group (p<0.05).

FIG. 6 shows that treatment with MPEP attenuates the CX614-induced decline in AMPAR subunit GluR expression. A. Photomicrographs of film autoradiograms showing GluR1 mRNA in a control hippocampal slice culture and following 48 h CX614 (20 μM) treatment. As shown, CX614 treatment reduced GluR1 mRNA levels. Co-treatment with CX614+MPEP blocked the decrease in GluR1 expression in all fields. B. Bar graph showing quantification of GluR1 mRNA levels in CA1 stratum pyramidale (CA1) of cultures treated 48 h with CX614 (20 μM), MPEP (50 μM) or a combination of both (n=12/group). Treatment with CX614 reduced GluR1 mRNA levels by 40% (p<0.01). However, in cultures co-treated with CX614+MPEP the decrease was blocked (p<0.01 for CX614+MPEP versus CX614 alone group). C. Bar graph showing quantification of GluR2 mRNA levels in CA1 stratum pyramidale (CA1) of cultures treated 48 h with CX614 (20 μM), MPEP (50 μM) or a combination of both (n=12/group). Treatment with CX614 reduced GluR2 mRNA levels nearly 50% (p<0.01). In cultures co-treated with CX614+MPEP the decrease was attenuated (p<0.05 for CX614+MPEP versus CX614 alone group). There was a small but non-significant increase with MPEP alone.

FIG. 7 shows that MPEP co-administration increases CX614-induced mature BDNF protein levels in organotypic hippocampal cultures. A. Western Blot analysis for mature BDNF protein in samples from control rat hippocampal slice cultures (“Con”) and cultures treated for 24 hours either with 50 μM CX614 (“CX614”), with 50 μM CX614 and 50 μM MPEP (“CX614+MPEP”) or with 50 μM MPEP. B. Quantification of optical densities from Western blots similar to those shown in panel A (n=5/group). Coadministration of CX614+MPEP leads to greater increase (25%) in total mature BDNF levels than CX614 alone. ***, p<0.0001 versus control group; *, p<0.05 for CX614 group versus CX614+MPEP group.

FIG. 8 shows the effect of CX929, an allosteric upmodulator of the AMPA receptor, on hippocampal total BNDF protein in vivo. Details are described in Example 8.

FIGS. 9A-9F show allosteric upmodulators of the AMPA receptor useful in the practice of this invention. Preferred compounds are indicated by numbers 1-54.

### DETAILED DESCRIPTION OF THE INVENTION

### I. Definitions

Unless defined otherwise, all technical and scientific terms used herein have the meaning commonly understood by a person skilled in the art to which this invention belongs.

The following references provide one of skill with a general definition of many of the terms used in this invention. Singleton et al., Dictionary of Microbiology and Molecular Biology (2nd ed. 1994); The Cambridge Dictionary of science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

As used herein “age-related sexual dysfunctions” are sexual dysfunctions that are manifested in aging subjects and that often worsen with increasing age. They are common to both human and animal species (Davidson et al., 1983, J Clin Endocrinol Metab 57(1):71-7; Smith and Davidson, 1990, Physiol Behav 47(4):631-4).

As used herein, the term “alkyl” refers to a straight or branched chain hydrocarbon radical, and can include di- and multivalent radicals, having the number of carbon atoms designated (i.e. C₁-C₁₀ means one to ten carbons). Examples of saturated hydrocarbon radicals include groups such as methyl, ethyl, n-propyl, isopropyl, n-butyl, t-butyl, isobutyl, sec-butyl, homologs and isomers of, for example, n-pentyl, n-hexyl, n-heptyl, n-octyl, and the like.

As used herein, the term “alkenyl” refers to an unsaturated alkyl group one having one or more double bonds. Examples of alkenyl groups include vinyl, 2-propenyl, crotyl, 2-isopentenyl, 2-(butadienyl), 2,4-pentadienyl and 3-(1,4-pentadienyl), and the higher homologs and isomers.

As used herein, the term “alkynyl” refers to an unsaturated alkyl group one having one or more triple bonds. Examples of alkynyl groups include ethynyl (acetylenyl), 1-propynyl, 1- and 2-butynyl, and the higher homologs and isomers.

As used herein, “allosteric upmodulator” means a compound which acts upon and increases the activity of an enzyme or receptor. The allosteric upmodulator does not act by directly stimulating neural activation, but by upmodulating (“allosteric modulation”) neural activation and transmission in neurons that contain glutamatergic receptors. For example, an allosteric upmodulator of an AMPA receptor increases ligand (glutamate) induced current flow (ion flux) through the receptor but has no effect on ion influx until the receptor's ligand is bound. Increased ion flux is typically measured as one or more of the following non-limiting parameters: at least a 10% increase in decay time, amplitude of the waveform and/or the area under the curve of the waveform and/or a decrease of at least 10% in rise time of the waveform, for example in preparations treated to block NMDA and GABA components. The increase or decrease is preferably at least 25-50%; most preferably it is at least 100%. How the increased ion flux is accomplished (for example, increased amplitude or increased decay time) is of secondary importance; up-modulation is reflective of increased ion fluxes through the AMPA channels, however achieved.

As used herein, “AMPA” refers to α-amino-3-hydroxy-5-methyl-4-isoxazolepropionic acid.

As used herein, “AMPAKINE®” refers to a group of benzamide type (benzoylpiperidine) drugs that enhance AMPA-receptor-gated currents. AMPAKINES® typically slow deactivation and/or desensitization of AMPA-type glutamate receptors and thereby increase ligand-gated current flow through the receptors (Arai et al., 1996, J Pharmacol Exp Ther 278:627-638; Arai et al., 2000, Mol Pharmacol 58:802-813). For example, an AMPAKINE® can function as an allosteric upmodulator for an AMP receptor.

As used herein, “α-amino-3-hydroxy-5-methyl-4-isoxazolepropionic acid receptor” or “AMPA receptor” refers to the class of glutamatergic receptors which are present in cells, particularly neurons, usually at their surface membrane that recognize and bind to glutamate or AMPA. AMPA receptors also bind kainite with moderate affinity. Typically, these receptors are oligomers composed of four homologous subunits (Boulter et al., 1990, Science 249:1033-1036; Keinanen et al., 1990, Science 249:556-560), each of which occurs as alternatively spliced isoforms “flip” or “flop” (Sommer et al., 1990, Science 249:1580-1585). Functional AMPA receptors can be built from each of the subunits alone and from virtually any combination of them. As each subunit imparts distinct biophysical properties to the receptors (Boulter et al., 1990, Science 249:1033-1036; Mosbacher et al., 1994, Science 266, 1059-1062) heterogeneity of AMPA receptor composition is likely to result in regional variations in the size and duration of excitatory postsynaptic currents (Bochet et al., 1994 Neuron 12:383-388; Geiger et al., 1995, Neuron 15:193-204; Arai and Lynch, 1996, Brain Res 716:202-206). The binding of AMPA or glutamate to an AMPA receptor normally gives rise to a series of molecular events or reactions that result in a biological response. The biological response may be the activation or potentiation of a nervous impulse, changes in cellular secretion or metabolism, causing the cells to undergo differentiation or movement, or increasing the level of a nucleic acid coding for a neurotrophic factor or a neurotrophic factor receptor.

As used herein, “antagonist” means a chemical substance that diminishes, abolishes or interferes with the physiological action of a ligand (agonist) that activates a receptor. Thus, the antagonist may be, for example, a chemical antagonist, a pharmacokinetic antagonist, an antagonist by receptor block, a non-competitive antagonist, or a physiological antagonist, such as a biomolecule, e.g., a polypeptide.

Specifically, a mGluR5 antagonist may act at the level of the ligand-mGluR5 interactions, such as by competitively or non-competitively (e.g., allosterically) inhibiting ligand binding. The antagonist may also act downstream of the mGluR5, such as by inhibiting mGluR5 interaction with a G protein. A “pharmacokinetic antagonist” effectively reduces the concentration of the active drug at its site of action, e.g., by increasing the rate of metabolic degradation of the active ligand. Antagonism by receptor-block involves two important mechanisms: (1) reversible competitive antagonism and (2) irreversible, or non-equilibrium, competitive antagonism. Reversible competitive antagonism occurs when the rate of dissociation of the antagonist molecule from the receptor is sufficiently high that, on addition of the ligand, the antagonist molecules binding the receptors are effectively replaced by the ligand. Irreversible or non-equilibrium competitive antagonism occurs when the antagonist dissociates very slowly or not at all from the receptor, with the result that no change in the antagonist occupancy takes place when the ligand is applied. Thus, the antagonism is insurmountable. A “competitive antagonist” is a molecule which binds directly to the receptor or ligand in a manner that sterically interferes with the interaction of the ligand with the receptor. Non-competitive antagonism describes a situation where the antagonist does not compete directly with ligand binding at the receptor, but instead blocks a point in the signal transduction pathway subsequent to receptor activation by the ligand. Physiological antagonism loosely describes the interaction of two substances whose opposing actions in the body tend to cancel each other out. An antagonist can also be a substance that diminishes or abolishes expression of functional mGluR. Thus, a mGluR5 antagonist can be, for example, a substance that diminishes or abolishes: (i) the expression of the gene encoding mGluR5, (ii) the translation of mGluR5 RNA, (iii) the post-translational modification of mGluR5 protein, or (iv) the insertion of mGluR5 into the cell membrane.

As used herein, a “selective mGluR5 antagonist” is an antagonist that antagonizes mGluR5, but antagonizes other mGluRs only weakly or substantially not at all, or at least antagonizes other mGluRs with an EC₅₀ at least 10 or even 100 or 1000 times greater than the EC₅₀ at which it antagonizes mGluR5. EC₅₀ means the effective concentration for 50% inhibition.

As used herein, “BDNF” means brain derived neurotrophic factor. Preferred is a BDNF from a human, BDNF may be from other mammals, not limited to, a non-human primate; a rodent, e.g., a mouse, a rat or hamster; cow, a pig, a horse, a sheep, or other mammal.

A “BDNF polypeptide” or “BDNF protein” includes both naturally occurring or recombinant forms. Therefore, in some embodiments, a BDNF polypeptide can comprise a sequence that corresponds to a human BDNF sequence. Exemplary BDNF polypeptide sequences are known in the art, for example, human BDNF (e.g., GenBank Accession Nos. CAA62632, P23560, AAO15434, AAL23571, and AAL23565), chimpanzee BDNF (e.g., GenBank Accession Nos. NP_{—}001012443 and AAV74288), mouse BDNF (e.g., GenBank Accession Nos. NP_{—}031566 and AAO74603), and rat BDNF (e.g., GenBank Accession Nos. NP_{—}036645 and AAH87634). A “BDNF” polypeptide includes BDNF variant polypeptides, e.g., translation products of an alternatively spliced BDNF nucleic acid.

A “BDNF nucleic acid” or “BDNF polynucleotide” refers to a vertebrate gene encoding a BDNF protein. A “BDNF nucleic acid” includes both naturally occurring or recombinant forms that can be either DNA or RNA. BDNF nucleic acids useful for practicing the present invention, have been cloned and characterized, for example, human BDNF (e.g., GenBank Accession Nos. X91251, AF411339, AT054406, and AY054400), chimpanzee BDNF (e.g., GenBank Accession Nos. NM_{—}001012441 and AY665250), mouse BDNF (e.g., GenBank Accession Nos. NM_{—}007540 and AY231132), and rat BDNF (e.g., GenBank Accession Nos. NM_{—}012513 and BC087634). A BDNF polynucleotide may be a full-length BDNF polynucleotide, i.e., encoding a complete BDNF protein or it may be a partial BDNF polynucleotide encoding a subdomain of a BDNF protein or it may be an alternatively spliced transcript encoding a variant polypeptide of BDNF.

As used herein, “biological sample” means a sample of biological tissue or fluid that contains nucleic acids or polypeptides. Such samples are typically from humans, but include tissues isolated from non-human primates, or rodents, e.g., mice, and rats. Biological samples may also include sections of tissues such as biopsy and autopsy samples, frozen sections taken for histological purposes, cerebral spinal fluid, blood, plasma, serum, sputum, stool, tears, mucus, hair, skin, etc. Biological samples also include explants and primary and/or transformed cell cultures derived from patient tissues. A “biological sample” also refers to a cell or population of cells or a quantity of tissue or fluid from an animal. Most often, the biological sample has been removed from an animal, but the term “biological sample” can also refer to cells or tissue analyzed in vivo, i.e., without removal from the animal. Typically, a “biological sample” will contain cells from the animal, but the term can also refer to noncellular biological material, such as noncellular fractions of cerebral spinal fluid, blood, saliva, or urine, that can be used to measure expression level of a polynucleotide or polypeptide. Numerous types of biological samples can be used in the present invention, including, but not limited to, a tissue biopsy or a blood sample. As used herein, a “tissue biopsy” refers to an amount of tissue removed from an animal, preferably a human, for diagnostic analysis. “Tissue biopsy” can refer to any type of biopsy, such as needle biopsy, fine needle biopsy, surgical biopsy, etc.

“Providing a biological sample” means to obtain a biological sample for use in methods described in this invention. Most often, this will be done by removing a sample of cells from a subject, but can also be accomplished by using previously isolated cells (e.g., isolated by another person, at another time, and/or for another purpose), or by performing the methods of the invention in vivo. Archival tissues, having treatment or outcome history, will be particularly useful.

As used herein, “blood-brain barrier permeant” or “blood-brain barrier permeable” means that at equilibrium the ratio of a compound's distribution in the cerebro-spinal fluid (CSF) relative to its distribution in the plasma (CSF/plasma ratio) is greater than 0.01, generally at least 0.02, preferably at least 0.05, and most preferably at least 0.1.

As used herein, “brain tissue” means individual or aggregates of cells from the brain. The cells may be obtained from cell culture of brain cells or directly from the brain or may be in the brain.

As used herein, “correlating the amount” means comparing an amount of a substance, molecule, marker, or polypeptide (such as a neurotrophic factor) that has been determined in one sample to an amount of the same substance, molecule, marker or polypeptide determined in another sample. The amount of the same substance, molecule, marker or polypeptide determined in another sample may be specific for a given disease or pathology.

As used herein, the term “cycloalkyl” refers to a saturated cyclic hydrocarbon having 3 to 15 carbons, and 1 to 3 rings that can be fused or linked covalently. Cycloalkyl groups useful in the present invention include, but are not limited to, cyclopentyl, cyclohexyl, cycloheptyl and cyclooctyl. Bicycloalkyl groups useful in the present invention include, but are not limited to, [3.3.0]bicyclooctanyl, [2.2.2]bicyclooctanyl, [4.3.0]bicyclononane, [4.4.0]bicyclodecane (decalin), spiro[3.4]octanyl, spiro[2.5]octanyl, and so forth.

As used herein, the term “cycloalkenyl” refers to an unsaturated cyclic hydrocarbon having 3 to 15 carbons, and 1 to 3 rings that can be fused or linked covalently. Cycloalkenyl groups useful in the present invention include, but are not limited to, cyclopentenyl, cyclohexenyl, cycloheptenyl and cyclooctenyl. Bicycloalkenyl groups are also useful in the present invention.

As used herein, the term “decreased expression” refers to the level of a gene expression product that is lower and/or the activity of the gene expression product is lowered. Preferably, the decrease is at least 20%, more preferably, the decrease is at least 30%, at least 40%, at least 50%, at least 60%, at least 70%, at least 80%, or at least 90% and most preferably, the decrease is at least 100%, relative to a control.

Synonyms of the term, “determining the amount” are contemplated within the scope of the present invention and include, but are not limited to, detecting, measuring, testing or determining, the presence, absence, amount or concentration of a molecule, such as a neurotrophic factor or small molecule of the invention, such as an AMPAKINE® or a mGluR5 antagonist.

As used herein, “determining the functional effect” means assaying for a compound that increases or decreases a parameter that is indirectly or directly under the influence of the compound, e.g., functional, enzymatic, physical and chemical effects. Such functional effects can be measured by any means known to those skilled in the art, e.g., changes in spectroscopic characteristics (e.g., fluorescence, absorbance, refractive index), hydrodynamic (e.g., shape), chromatographic, or solubility properties for the protein, measuring inducible markers or transcriptional activation of a neurotrophic factor encoding gene; measuring binding activity, e.g., binding of a neurotrophic factor to a neurotrophic factor receptor, measuring cellular proliferation, measuring apoptosis, or the like. Determination of the functional effect of a compound on a disease, disorder, cancer or other pathology can also be performed using assays known to those of skill in the art such as an in vitro assays, e.g., cellular proliferation; growth factor or serum dependence; mRNA and protein expression in cells, and other characteristics of cells. The functional effects can be evaluated by many means known to those skilled in the art, e.g., microscopy for quantitative or qualitative measures of alterations in morphological features, measurement of changes in neurotrophic factor RNA or protein levels, measurement of RNA stability, identification of downstream or reporter gene expression (CAT, luciferase, β-gal, GFP and the like), e.g., via chemiluminescence, fluorescence, colorimetric reactions, antibody binding, inducible markers, and ligand binding assays. “Functional effects” include in vitro, in vivo, and ex vivo activities.

As used herein, “diminish the symptoms of sexual dysfunction” means denotes a decrease in the inhibition of any one or more of the four phases of sexual response (appetite, excitement, orgasm, resolution) described in the DSM-IIIR. The phrase specifically encompasses increased sexual desire, the enhanced ability to sustain a penile erection, the enhanced ability to ejaculate and/or to experience orgasm. A particular example of diminished symptoms of sexual dysfunction is an increase in the number, frequency and duration of instances of sexual behavior or of subjective sexual arousal.

As used herein, “disorder” and “disease” are used inclusively and refer to any deviation from the normal structure or function of any part, organ or system of the body (or any combination thereof). A specific disease is manifested by characteristic symptoms and signs, including biological, chemical and physical changes, and is often associated with a variety of other factors including, but not limited to, demographic, environmental, employment, genetic and medically historical factors. Certain characteristic signs, symptoms, and related factors can be quantitated through a variety of methods to yield important diagnostic information.

As used herein, “endocrine system” refers in general to the hormonal cell-cell communication system of a mammal. By “modulation of the endocrine system” is meant that the hormonal cell-cell communication of the mammal is altered in some manner, usually through a modulation or change in the blood circulatory level of one or more endogenous hormones, where modulation includes both increasing and decreasing the circulatory level of one or more hormones, usually increasing the circulatory level of one or more hormones, in response to the administration of an AMPAKINE® and a mGluR5 antagonist. Usually the subject methods are employed to modulate the activity of a particular hormonal system of the endocrine system of the mammal, where hormonal systems of interest include those which comprise glutamatergic regulation, particularly AMPA receptor regulation, where the hypothalamus-pituitary hormonal system is of particular interest.

As used herein, “effective amount”, “effective dose”, sufficient amount”, “amount effective to”, “therapeutically effective amount” or grammatical equivalents thereof mean a dosage sufficient to produce a desired result, to ameliorate, or in some manner, reduce a symptom or stop or reverse progression of a condition. In some embodiments, the desired result is an increase in neurotrophic factor expression or neurotrophic factor receptor expression. Amelioration of a symptom of a particular condition by administration of a pharmaceutical composition described herein refers to any lessening, whether permanent or temporary, lasting or transit that can be associated with the administration of the pharmaceutical composition. An “effective amount” can be administered in vivo and in vitro.

As used herein, the term “halogen” refers to the elements including fluorine (F), chlorine (Cl), bromine (Br) and iodine (I).

As used herein, the term “heteroaryl” refers to a polyunsaturated, aromatic, hydrocarbon substituent having 5-12 ring members, which can be a single ring or multiple rings (up to three rings) which are fused together or linked covalently, and which has at least one heteroatom in the ring, such as N, O, or S. A heteroaryl group can be attached to the remainder of the molecule through a heteroatom. Non-limiting examples of heteroaryl groups include 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Additional heteroaryl groups useful in the present invention include pyridyl N-oxide, tetrazolyl, benzofuranyl, benzothienyl, indazolyl, or any of the radicals substituted, especially mono- or di-substituted.

As used herein, the term “heterocycloalkyl” refers to a saturated cyclic hydrocarbon having 3 to 15 ring members, and 1 to 3 rings that can be fused or linked covalently, and which has at least one heteroatom in the ring, such as N, O, or S. Additionally, a heteroatom can occupy the position at which the heterocycle is attached to the remainder of the molecule. Examples of heterocycloalkyl include 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

As used herein, “improving a cognitive function” or “improvement of a cognitive function” means increasing the capacity of the subject to perform the cognitive function. The terms also refer to an increased or improved baseline level of the cognitive function in the subject and to an increased or improved level of the cognitive function in response to a challenge or test. A “reduced cognitive activity” refers to a cognitive activity or cognitive function below a baseline level in a subject. It also refers to a cognitive function performed by a subject at a lower level than the cognitive function performed by a healthy or unaffected subject.

As used herein, “increasing the expression” or “increased expression” or similar grammatical equivalents refers to the level of a gene expression product that is made higher and/or the activity of the gene expression product is enhanced. Preferably, the increase is by at least 25%. More preferably the increase is at least 1-fold, at least 2-fold, at least 5-fold, or at least 10-fold, and most preferably, the increase is at least 20-fold, relative to a control. In reference to a particular protein the terms also mean to cause a detectable increase in the amount of an mRNA encoding the referenced protein. Typically, the transcription product assayed for is mRNA. An increase in transcription product may be caused by any number of means including increased transcription rate or decreased degradation rate.

As used herein, “increasing the level” in reference to a particular compound, means to cause a detectable increase in the amount of the referenced compound.

As used herein, “in need of increased neurotrophic factor” or “in need of increased neurotrophic factor receptor” means a clinically assessed need to inhibit, suspend, or mitigate the progression or occurrence of a pathology which produces neurodegeneration or sublethal neuronal pathology and to which end an increase in neurotrophic factor or neurotrophic factor receptor in the brain is recommended by one of skill in the art of treating the particular pathology.

As used herein, the term “isomers” refers to compounds of the present invention that possess asymmetric carbon atoms (optical centers) or double bonds. The racemates, diastereomers, geometric isomers and individual isomers are all intended to be encompassed within the scope of the present invention.

As used herein, “in vitro” means outside the body of the organism from which a cell or cells is obtained or from which a cell line is isolated.

As used herein, “in vivo” means within the body of the organism from which a cell or cells is obtained or from which a cell line is isolated.

A “label” or a “detectable moiety” is a composition detectable by spectroscopic, photochemical, biochemical, immunochemical, chemical, or other physical means. For example, useful labels include ³H, ¹²⁵I, ³²P, fluorescent dyes, electron-dense reagents, enzymes (e.g., as commonly used in an ELISA), biotin, digoxigenin, or haptens and proteins or other entities which can be made detectable, e.g., by incorporating a radiolabel into a small molecule compound. A label may be incorporated into a small molecule compound, such as an AMPAKINE® or mGluR5 antagonist, at any position.

As used herein, “level of a mRNA” in a biological sample refers to the amount of mRNA transcribed from a gene that is present in a cell or a biological sample. The mRNA generally encodes a functional protein, although mutations may be present that alter or eliminate the function of the encoded protein. A “level of mRNA” need not be quantified, but can simply be detected, e.g., a subjective, visual detection by a human, with or without comparison to a level from a control sample or a level expected of a control sample. A preferred mRNA is a BDNF mRNA.

As used herein, “level of a polypeptide” in a biological sample refers to the amount of polypeptide translated from a mRNA that is present in a cell or biological sample. The polypeptide may or may not have protein activity. A “level of a polypeptide” need not be quantified, but can simply be detected, e.g., a subjective, visual detection by a human, with or without comparison to a level from a control sample or a level expected of a control sample. A preferred polypeptide is a BDNF polypeptide.

As used herein, “mammal” or “mammalian” means or relates to the class mammalia including the orders carnivore (e.g., dogs and cats). rodentia (e.g., mice. guinea pigs, and rats), and primates (e.g., humans, chimpanzees, and monkeys).

As used herein, “metabotropic glutamate receptor” or “mGluR” refers to a group of G-protein-coupled receptors that are further subgrouped into (i) group 1 mGluR, including mGluR1 and mGluR5, (ii) group II mGluR, including mGluR2, and mGluR3, and (iii) group III mGluR, including mGluR4, mGluR6, mGluR7, and mGluR8. Thus, for example, “mGluR1” refers to metabotropic glutamate receptor 1 and “mGluR5” refers to metabotropic glutamate receptor 5.

As used herein “mood” means an individual's enduring emotional state, while “affect” refers to short-term fluctuations in emotional state. Thus, the term “mood disorder” is used in reference to conditions in which abnormalities of emotional state are the core symptoms. The most common serious mood disorders reportedly seen in general medical practice are major depression (unipolar depression), dysthymic disorder (chronic, milder form of depression), and bipolar disorder (manic-depressive illness).

As used herein, “neurotrophic factor” means a polypeptide that supports the growth, differentiation, and survival of neurons in the developing nervous system and maintains neurons and their biosynthetic activities in the mature nervous system. Exemplary neurotrophic factors include, but are not limited to, (i) neurotrophins (e.g., nerve growth factor (NGF), brain-derived neurotrophic factor (BDNF), neurotrophin-3 (NT-3), neurotrophin-4/5 (NT-4/5)), (ii) neuropoietins (e.g., ciliary neurotrophic factor (CNTF), (iii) leukemia inhibitory factor (LIF)), (iv) insulin-like growth factors (e.g. insulin-like growth factor-1 (IGF-1), insulin-like growth factor-II (IGF-II)), (v) transforming growth factor beta (e.g., transforming growth factor β (TGFβ₁, TGFβ₂, TGFβ₃)), (vi) fibroblast growth factors (e.g. acidic fibroblast growth factor (aFGF), basic fibroblast growth factor (bFGF), fibroblast growth factor-5 (FGF-5)), and (vii) others such as transforming growth factor alpha (TGF-α), platelet-derived growth factor (PDGF: AA, AB, and BB isoforms), epidermal growth factor (EGF), glial cell-derived neurotrophic factor (GDNF), and stem cell factor.

As used herein, “neurotrophic factor receptor” means a receptor which acts as a target for a neurotrophic factor including, but not limited to, the Trk family (e.g., TrkA, TrkB, and TrkC); the CNTF receptor complex (e.g., CNTFRα, gp130, LIFRβ); LIF receptor complex (e.g., gp130, LIFRP); IGF Type 1 receptor; insulin receptor; TGFβ type I, II, and III receptors; GFG receptors 1-4; epidermal growth factor receptor (EGFR); PDGF α- and β-receptors; GDNF family receptor alpha and Ret; and c-kit.

As used herein, “pathology which produces neurodegeneration” means a disease, metabolic disorder, direct physical or chemical insult, or any physiological process causing or participating in neuronal injury or death.

As used herein, “pharmaceutically acceptable” refers to compositions that are physiologically tolerable and do not typically produce an allergic or similar untoward reaction when administered to a subject, preferably a human subject. Preferably, as used herein, the term “pharmaceutically acceptable” means approved by a regulatory agency of a Federal or state government or listed in the U.S. Pharmacopeia or other generally recognized pharmacopeia for use in animals, and more particularly in humans.

As used herein, “polypeptide,” and “protein” are used interchangeably herein to refer to a polymer of amino acid residues.

As used herein, “providing a biological sample” means to obtain a biological sample for use in methods described in this invention. Most often, this will be done by removing a sample of cells from a patient, but can also be accomplished by using previously isolated cells (e.g., isolated by another person, at another time, and/or for another purpose), or by performing the methods of the invention in vivo. Archival tissues, having treatment or outcome history, will be particularly useful.

As used herein “neuropsychiatric condition” or “neuropsychiatric disorder” mean mental, emotional, or behavioral abnormalities. These include, but are not limited to, bipolar disorder, schizophrenia, schizoaffective disorder, psychosis, depression, stimulant abuse, alcoholism, panic disorder, generalized anxiety disorder, attention deficit disorder, post-traumatic stress disorder, Parkinson's disease, Alzheimer's disease, cognitive impairment, mental retardation, Fragile X, and autism.

The terms “psychotic” and “psychiatric” arte used interchangeably.

As used herein, the term “salts” refers to salts of the active compounds of the present invention, such as AMPAKINES® or mGluR5 antagonists, which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge, S. M., et al, “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

As used herein, “schizophrenia” means Schizophrenia or Schizophreniform Disorder or Schizoaffective Disorder or Delusional Disorder or Brief Psychotic Disorder or Psychotic Disorder Due to a General Medical Condition or Psychotic Disorder Not Otherwise Specified, and the symptoms of these disorders, are in large part as defined in the Diagnostic and Statistical Manual of Mental Disorder, fourth edition (DSMIV). The sections of the DSMIV that relate to these disorders are hereby incorporated by reference.

As used herein, “sexual dysfunction” means the inhibition of any one or more of the phases of sexual response (appetite, excitement, orgasm, resolution) described in the DSM-IIIR. “Sexual dysfunction” specifically encompasses decreased sexual desire (Hypoactive Sexual Desire Disorder, DSM-III-R #302.71), the inability to sustain a penile erection (Male Erectile Disorder, DSM-III-R #302.72), the inability to ejaculate and/or the inability to experience orgasm (Inhibited Male Orgasm, DSM-III-R #302.74). All may be psychogenic only, or psychogenic and biogenic, lifelong or acquired, and generalized or situational. The DSM-IIIR definitions and text relating to sexual dysfunction are hereby incorporated by reference.

As used herein, the term “solvate” refers to compounds of the present invention that are complexed to a solvent. Solvents that can form solvates with the compounds of the present invention include common organic solvents such as alcohols (methanol, ethanol, etc.), ethers, acetone, ethyl acetate, halogenated solvents (methylene chloride, chloroform, etc.), hexane and pentane. Additional solvents include water. When water is the complexing solvent, the complex is termed a “hydrate.”

As used herein, “subject” or “patient” to be treated for a condition or disease by a subject method means either a human or non-human animal in need of treatment for a condition or disease.

As used herein, “symptoms of sexual dysfunction” includes inhibition of any of the four phases of sexual response (appetite, excitement, orgasm, resolution) mentioned in the DSM-IIIR. These specifically include lack of sexual desire (Hypoactive Sexual Desire Disorder, DSM-III-R #302.71), impotence or the inability to sustain a penile erection (Male Erectile Disorder, DSM-III-R #302.72), the inability to ejaculate and/or the inability to experience orgasm (Inhibited Male Orgasm, DSM-III-R #302.74).

As used herein, the terms “treat”, “treating”, and “treatment” include: (1) preventing a condition or disease, i.e. causing the clinical symptoms of the condition or disease not to develop in a subject that may be predisposed to the condition or disease but does not yet experience any symptoms of the condition or disease; (2) inhibiting the condition or disease, i.e. arresting or reducing the development of the condition or disease or its clinical symptoms; or (3) relieving the condition or disease, i.e. causing regression of the condition or disease or its clinical symptoms. These terms encompass also prophylaxis, therapy and cure. Treatment means any manner in which the symptoms or pathology of a condition, disorder, or disease are ameliorated or otherwise beneficially altered. Preferably, the subject in need of such treatment is a mammal, more preferable a human.

### II. Small Molecule Compounds

A. Positive AMPA Receptor Modulators

Applicants describe herein novel approaches for the treatment of neurological and neuropsychiatric disorders, wherein AMPA receptors are modulated using both a positive AMPA receptor modulator, i.e., an AMPAKINE®, and a group 1 mGluR5 antagonist. As described herein, it is an objective of the present invention to provide AMPAKINES® useful to practice the methods of the present invention.

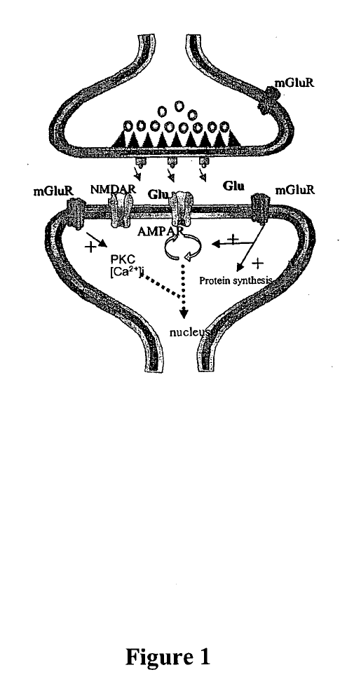

Compounds useful in the practice of this invention are generally those that amplify the activity of the natural stimulators of AMPA receptors particularly by amplifying excitatory synaptic response, as defined herein, i.e. an allosteric upmodulator of an AMPA receptor. Allosteric upmodulator of AMPA receptors that find use in the subject invention include the “AMPAKINES” described: in WO 94/02475 (PCT/US93/06916); U.S. Pat. Nos. 5,650,409, 6,329,368; as well as WO98/12185; the disclosures of which applications are expressly incorporated herein by reference. Particular compounds of interest include: aniracetam, 7-chloro-3-methyl-3-4-dihydro-2H-1,2,4 benzothiadiazine S,S, dioxide, (see Zivkovic et al., 1995, J Pharmacol Exp. Therap 272:300-309; Thompson et al., 1995, Proc Nat Acad Sci USA 92:7667-7671) and those compounds shown in FIGS. 1A-1E of U.S. Pat. No. 6,030,968, expressly incorporated herein by reference. The compounds disclosed in the literature and patents cited above can be prepared by conventional methods known to those skilled in the art of synthetic organic chemistry.

AMPAKINES® typically slow deactivation and/or desensitization of AMPA-type glutamate receptors and thereby increase ligand-gated current flow through the receptors (Arai et al., 1996, J Pharmacol Exp Ther 278:627-638; Arai et al., 2000, Mol Pharmacol 58:802-813). AMPAKINES® are of particular interest with regard to potential neurotrophin-based treatments because they (i) readily cross the blood-brain barrier (Staubli et al., 1994, Proc Natl Acad Sci USA 91:777-781); (ii) are orally active (Lynch et al., 1997, Exp Neurol 145:89-92; Goff et al., 2001, J Clin Psychopharmacol 21:484-487); (iii) have subtle and seemingly positive effects on behavior (Lynch et al., 2002, Nat Neurosci 5:1035-1038); and (iv) in preliminary studies, improved cognitive function in humans without evident side effects (Lynch et al., 1997, Exp Neurol 145:89-92; Lynch et al., 2002, Nat Neurosci 5:1035-1038).

AMPAKINES® useful for practicing the present invention are well described in the scientific and patent literature. For example, structures, synthesis, formulations and assays for the AMPAKINES® detailed herein and of additional AMPAKINES®, useful to practice the present invention, are disclosed, for example, in U.S. Pat. Nos. 5,747,492, 5,773,434, 5,852,008, 5,891,876, 6,030,968, 6,083,947, 6,166,008, 6,274,600, and 6,329,368, which are incorporated in their entirety by reference. Certain groups of these compounds fall within generic structural classes, e.g., as those described in U.S. Pat. No. 5,773,434. Heteroatom substituted benzoyl derivatives, useful to practice the present invention, are described, for example in U.S. Pat. Nos. 5,747,492, 5,852,008, 5891,876, and 6,274,600.

AMPAKINES®, R,S-α-amino-3-hydroxy-5-methyl-4-isoxazolepropionic acid (AMPA) receptor upmodulators of the benzamide type, have previously been shown to enhance excitatory synaptic transmission in vivo and in vitro and AMPA receptor currents in excised patches.

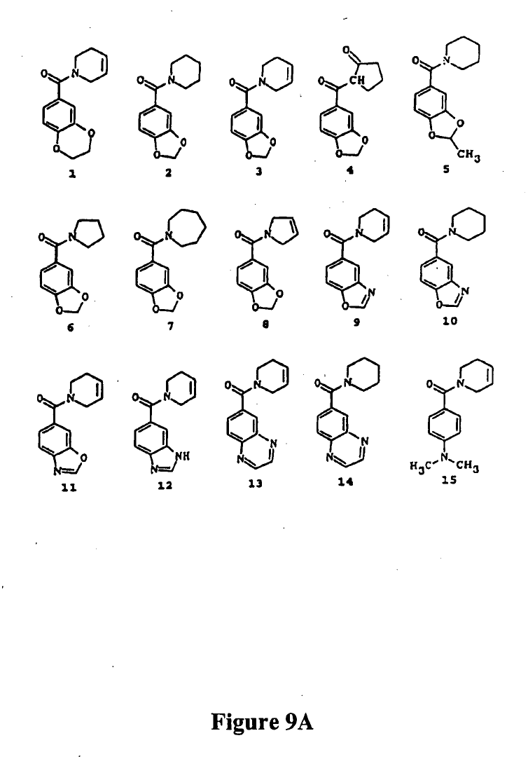

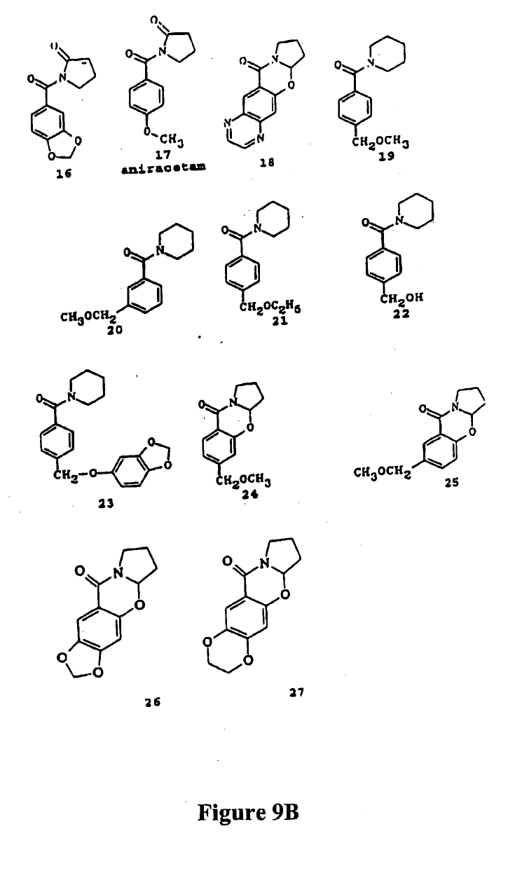

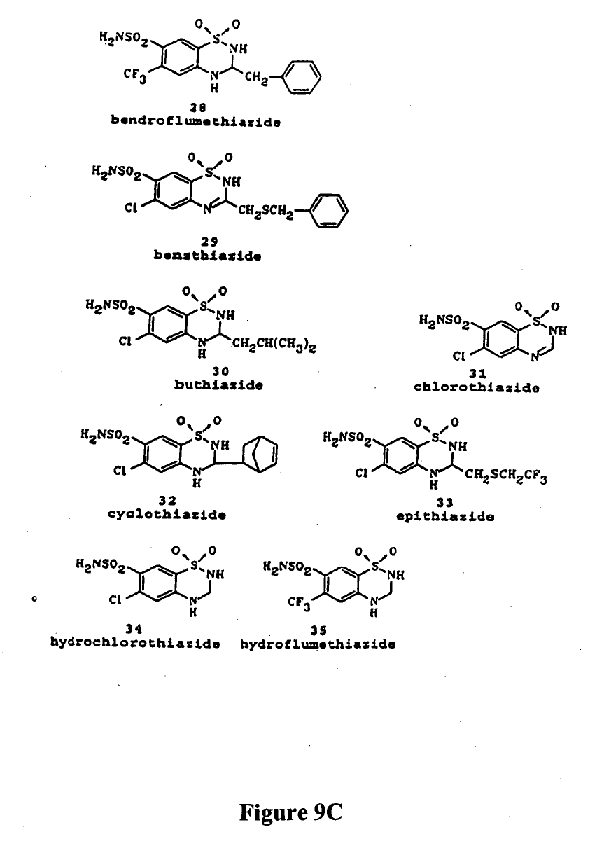

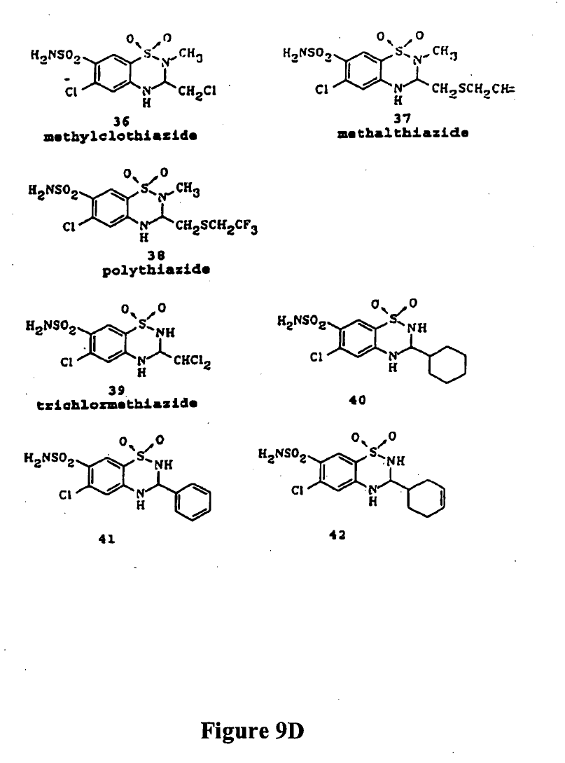

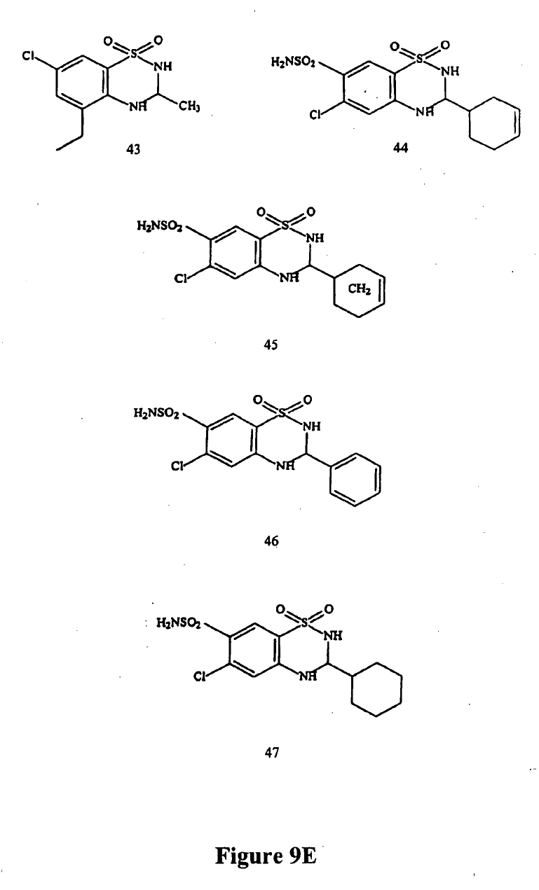

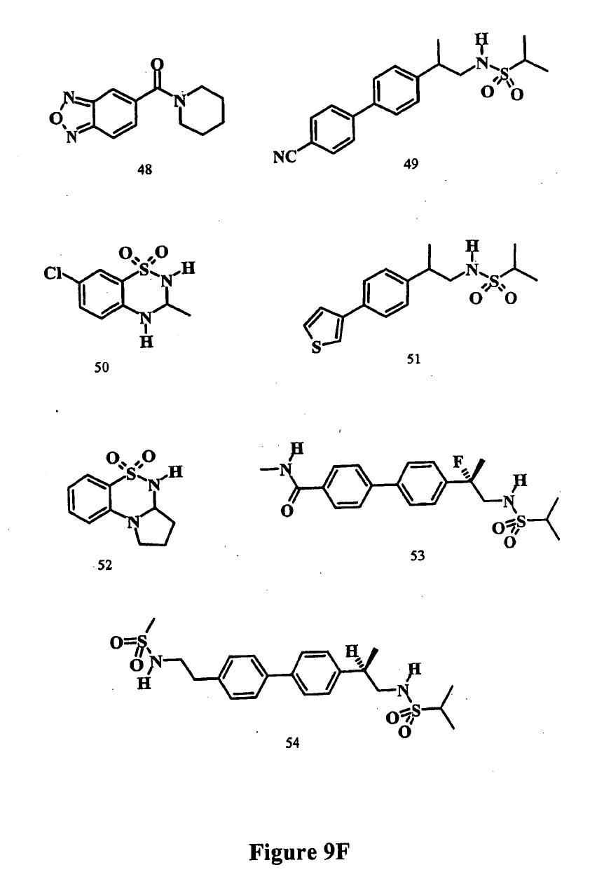

In a preferred embodiment of the present invention, the AMPA-receptor allosteric upmodulator is selected from the group of compounds 1-54 depicted in FIGS. 9A-9F.

In another preferred embodiment of the present invention, the AMPA-receptor allosteric upmodulator is a compound for which the structure is depicted in FIG. 9. Thus, a preferred AMPA-receptor allosteric upmodulator is compound 1, compound 2, compound 3, compound 4, compound 5, compound 6, compound 7, compound 8, compound 9, compound 10, compound 11, compound 12, compound 13, compound 14, compound 15, compound 16, compound 17, compound 18, compound 19, compound 20, compound 21, compound 22, compound 23, compound 24, compound 25, compound 26, compound 27, compound 28, compound 29, compound 30, compound 31, compound 32, compound 33, compound 34, compound 35 compound 36, compound 37, compound 38, compound 39, compound 40, compound 41, compound 42, compound 43, compound 44, compound 45 compound 46, compound 47, compound 48, compound 49, compound 50, compound 51, compound 52, compound 53, compound 54, or a structural analog thereof. Also, stereoisomers thereof, or pharmaceutically acceptable salts or hydrates thereof can be used to practice this invention.

In another preferred embodiment of the present invention, the AMPA-receptor allosteric upmodulator is selected from the group consisting of CX516, CX546, CX614, CX691, CX717, CX929, and structural analogs thereof.

In another preferred embodiment, the AMPA-receptor allosteric upmodulator is CX516. Thus, also preferred for use in the present invention is 1-(quinoxalin-6-ylcarbonyl)piperidine (CX516; Cortex Pharmaceuticals Inc.; Arai et al., 2004, Neuroscience 123(4):1011-24), an AMPAKINE® for the potential treatment of Alzheimer's disease, schizophrenia, mild cognitive impairment, attention deficit hyperactivity disorder, and fragile X syndrome (Goff et al., 2001, J Clin Psychopharmacol 21 (5):484-7; Danysz, 2002, Curr Opin Investig Drugs 3(7): 1062-6; Danysz, 2002, Curr Opin Investig Drugs 3(7): 1081-8). Preclinical and pilot clinical studies have shown that CX516 has the ability to enhance memory and cognition (Johnson and Simmon, 2002, J Mol Neurosci 19(1-2): 197-200). In another study, CX516 has been used as a sole agent in a limited double blind placebo-controlled study in patients with schizophrenia, however, did not appear to yield dramatic effects at the doses tested (Marenco et al., 2002, Schizophr Res 57(2-3):221-6). CX516 is currently evaluated for an Alzheimer's disease treatment (Doraiswamy and Xiong, 2006, Expert Opin Pharmacother 7(1):1-10).

In another preferred embodiment, the AMPA-receptor allosteric upmodulator is CX546. The AMPAKINE® CX546 1-(1,4-benzodioxan-6-ylcarbonyl)piperidine (CX546; Cortex Pharmaceuticals Inc.) has been reported to reduce the desensitization of AMPA receptors more potently than CX516 (Nagarajan et al., 2001, Neuropharamacology 41(6):650-63); Arai et al., 2004, Neuroscience 123(4):1011-24).

In another preferred embodiment, the AMPA-receptor allosteric upmodulator is CX614. The preferred AMPAKINE® CX614 (2H,3H,6aH-pyrrolidino[2″,1″-3′,2′]1,3-oxazino[6′,5′-5,4]benzo[e]1,4-dioxan-10-one; Cortex Pharmaceuticals Inc.) belongs to a benzoxazine subgroup characterized by great structural rigidity and high potency. CX614 is also referred to as LiD37 (listed as compound 27 in U.S. Pat. No. 6,030,968 and as compound 27 in FIG. 9).

In another preferred embodiment, the AMPA-receptor allosteric upmodulator is CX691. The structure of compound CX691 is shown as compound 48 in FIG. 9F.

In another preferred embodiment, the AMPA-receptor allosteric upmodulator is CX717. In addition to enhancing cognitive performance under normal alert conditions, the AMPAKINE® CX717 (Cortex Pharmaceuticals Inc.) also proved effective in non-human primates to alleviate the impairment of performance due to sleep deprivation (Porrino et al., 2005, PLos Biol 3(9):e299).

In another preferred embodiment, the AMPA-receptor allosteric upmodulator is CX929.

Another preferred AMPAKINE® is DP75 (see, U.S. Pat. No. 6,030,968).

In addition, the salts, hydrates, solvates, isomers and prodrugs of the AMPAKINES® described herein are also contemplated for use in the method of the present invention.

B. Group 1 Metabotropic Glutamate Receptor 5 Antagonists

Group I metabotropic glutamate receptors include the metabotropic glutamate receptor 1 (mGluR1) and the metabotropic glutamate receptor 5 (mGluR5). Antagonists to each mGluR1 and mGluR5 are known in the art. The effects of mGluR1 antagonists may be qualitatively different from those of mGluR5 antagonists and may depend on the experimental procedure (see, e.g., Pietraszek et al., 2005, Eur J Pharmacol 514(1):25-34). However, none of them has been identified to work in synergism with an AMPAKINE® as described herein.

The present invention contemplates the use of an AMPAKINE® and a group I mGluR antagonist, preferably a mGluR5 antagonist, for increasing the expression of a neurotrophic factor above the level obtained with an AMPAKINE® alone. Thus, it is an objective of the present invention to provide mGluR5 antagonists useful to practice the methods of the present invention. In a preferred embodiment of the present invention, the mGluR5 antagonist is a selective mGluR5 antagonist.

Exemplary mGluR5 antagonists include, without limitation, 2-methyl-6-(phenylethynyl)-pyridine (MPEP), (E)-2-methyl-6-styryl-pyridine (SIB 1893), LY293558, 2-methyl-6-[(1E)-2-phenylethynyl]-pyridine, 6-methyl-2-(phenylazo)-3-pyridinol, (RS)-α-methyl-4-carboxyphenylglycine (MCPG), 3S,4aR,6S,8aRS-6-((((1H-tetrazole-5-yl)methyl)oxy)methyl)-1,2,3,4,4a,5,6,7,8,8a-decahydroisoquinoline-3-carboxylic acid, 3S,4aR,6S,8aR-6-((((1H-tetrazole-5-yl)methyl)oxy)methyl)-1,2,3,4,4a,5,6,7,8,8a-decahydroisoquinoline-3-carboxylic acid, 3SR,4aRS,6SR,8aRS-6-(((4-carboxy)phenyl)methyl)-1,2,3,4,4a,5,6,7,8,8a-decahydroisoquinoline-3-carboxylic acid and 3S,4aR,6S,8aR-6-(((4-carboxy)-phenyl)methyl)-1,2,3,4,4a,5,6,7,8,8a-decahydroisoquinoline-3-carboxylic acid, and their pharmaceutically acceptable salts, analogues and derivatives thereof.

Thus, in one embodiment of the present invention, a mGluR5 antagonists is selected from the group consisting of 2-methyl-6-(phenylethynyl)-pyridine (MPEP), (E)-2-methyl-6-styryl-pyridine (SIB 1893), LY293558, 2-methyl-6-[(1E)-2-phenylethynyl]-pyridine, 6-methyl-2-(phenylazo)-3-pyridinol, (RS)-α-methyl-4-carboxyphenylglycine (MCPG), 3S,4aR,6S,8aRS-6-((((1H-tetrazole-5-yl)methyl)oxy)methyl)-1,2,3,4,4a,5,6,7,8,8a-decahydroisoquinoline-3-carboxylic acid, 3S,4aR,6S,8aR-6-((((1H-tetrazole-5-yl)methyl)oxy)methyl)-1,2,3,4,4a,5,6,7,8,8a-decahydroisoquinoline-3-carboxylic acid, 3SR,4aRS,6SR,8aRS-6-(((4-carboxy)phenyl)methyl)-1,2,3,4,4a,5,6,7,8,8a-decahydroisoquinoline-3-carboxylic acid and 3S,4aR,6S,8aR-6-(((4-carboxy)-phenyl)methyl)-1,2,3,4,4a,5,6,7,8,8a-decahydroisoquinoline-3-carboxylic acid, and their pharmaceutically acceptable salts, analogues and derivatives thereof.

A preferred mGluR5 antagonist for use in the present invention is the noncompetitive antagonist MPEP (2-methyl-6-(phenylethynyl)pyridine).

Another preferred mGluR5 antagonist for practicing the present invention is SIB-1893 [(E)-2-methyl-6-styryl-pyridine] is a structural analog of MPEP.

Recently, other close structural analogs of MPEP that bind to the MPEP site on mGluR5 were described. These compounds are also useful for practicing the present invention and include M-5MPEP [2-(2-(-methoxyphenyl)ethynyl)-5-methylpyridine], Br-5MPEPy [2-(2-(5-bromopyridin-3-yl)ethynyl)-5-methylpyridine, and 5 MPEP (5-methyl-6-(phenylethynyl)-pyridine) (Rodriguez et al., 2005, Mol Pharmacol 68(6): 1793-802). While M-5MPEP and Br-5MPEPy partially inhibited the response of mGluR5 to glutamate, no functional effect attributed to 5 MPEP alone on the mGluR5 response was described. However, 5 MPEP blocked the effect of both MPEP and potentiators (Rodriguez et al., 2005, Mol Pharmacol 68(6): 1793-802).

MTEP (3-[(2-methyl-1,3-thiazol-4-yl)ethynyl]pyridine; Varty et al., 2005, Psychopharmacology (Berl) 179(1):207-17) is another preferred mGluR5 antagonist that can be used in the method and compositions of the present invention. It has been shown to have anxiolytic activity in rats and has been reported to be 5-fold more potent than MPEP in the rat fear-potentiated startle model of anxiety (Cosford et al., 2003, J Med Chem 46(2):204-6). MTEP significantly reduced fear-potentiated startle and increased punished responding consistent with an anxiolytic-like profile (Busse et al., 2004, Neuropsychopharmacology 29(11):1971-9).

Other recently identified analogues of MTEP with high potency as mGluR5 antagonist and useful to practice the present invention have been described by Iso et al. (2006, J Med Chem 49(3): 1080-100). These compounds include (number in parentheses corresponds to compound #): 2-methyl-4-(trimethylsilylethynyl)thiazole (4), 2,5-dimethyl-4-(trimethylsilylethynyl)thiazole (5), 5-ethyl-2-methyl-4-(trimethylsilylethynyl)thiazole (6), 1-phenyl-4-trimethylsilyl-3-butyn-2-one (7),2-methyl-5-phenyl-4-(trimethylsilylethynyl)thiazole (9), 4-(3-fluorophenylethynyl)-2-methylthiazole (10), 4-(4-fluorophenylethynyl)-2-methylthiazole (11), 4-(3-methoxyphenylethynyl)-2-methylthiazole (12), 4-(2-fluorophenylethynyl)-2-methylthiazole (13), 4-2-methoxyphenylethynyl)-2-methylthiazole (14), 2-methyl-4-(m-tolylethynyl)thiazole (15), 4-(3-chlorophenylethynyl)-2-methylthiazole (16), 2-methyl-4-[[3-(trifluoromethyl)phenyl]ethynyl]thiazole (17), 2-methyl-4-[[3-(trifluoromethoxy)phenyl]ethynyl]thiazole (18), 3-[(2-methyl-4-thiazolyl)ethynyl]benzonitrile (19), N-[3-[(2-methyl-4-thiazolyl)ethynyl]phenyl]acetamide (20), 4-(3,5-difluorophenylethynyl)-2-methylthiazole (21), 3-[(2,5-dimethyl-4-thiazolyl)ethynyl]pyridine (23), 3-[(5-ethyl-2-methyl-4-thiazolyl)ethynyl]pyridine (24), 3-[(2-methyl-5-phenyl-4-thiazolyl)ethynyl]pyridine (25), 2-methoxy-5-[(2-methyl-4-thiazolyl)ethynyl]pyridine (26), 5-fluoro-2-[(2-methyl-4-thiazolyl)ethynyl]pyridine (27), 3-bromo-5-[(2-methyl-4-thiazolyl)ethynyl]pyridine (28), 2-fluoro-5-[(2-methyl-4-thiazolyl)ethynyl]pyridine (29), 5-[(2-methyl-4-thiazolyl)ethynyl]pyrimidine (30), 2-[(2-methyl-4-thiazolyl)ethynyl]pyrazine (31), 2-methyl-4-(2-thienylethynyl)thiazole (32), 2-methyl-4-(3-thienylethynyl)thiazole (33), 3-[(2-methyl-4-thiazolyl)ethynyl]quinoline (34), 6-[(2-methyl-4-thiazolyl)ethynyl]quinoxaline (35), 5-[(2-methyl-4-thiazolyl)ethynyl]-1H-indole (36), 3-[(2-methyl-4-thiazolyl)ethynyl]phenol (37), 3-[(2-methyl-4-thiazolyl)ethynyl]benzamide (38), 4-(trimethylsilylethynyl)-2-thiazolylamine (39), 4-(3-fluorophenylethynyl)-2-thiazolylamine (40), 4-(3-pyridylethynyl)-2-thiazolylamine (41), N-[4-(3-pyridylethynyl)-2-thiazolyl]acetamide (42), N-[4-(3-pyridylethynyl)-2-thiazolyl]benzamide (43), 1-(2,4-difluorophenyl)-3-[4-(3-pyridylethynyl)-2-thiazolyl]urea (44), [4-(3-pyridylethynyl)-2-thiazolyl]carbamic acid methyl ester (45), 2-bromo-4-(3-fluorophenylethynyl)thiazole (46), 2-(3,5-difluorophenyl)-4-(3-fluorophenylethynyl)thiazole (47), 3-[4-(3-fluorophenylethynyl)-2-thiazolyl]-2-propyn-1-ol (49), 2-ethynyl-4-(3-fluorophenylethynyl)thiazole (50), 3-(4-fluorophenyl)-5-[(2-methyl-4-thiazolyl)ethynyl]pyridine (52), 3-(4-methoxyphenyl)-5-[(2-methyl-4-thiazolyl)ethynyl]pyridine (53), 3-[5-[(2-methyl-4-thiazolyl)ethynyl]-3-pyridyl]-2-propyn-1-ol (55), 3-ethynyl-5-[(2-methyl-4-thiazolyl)ethynyl]pyridine (56), 3-[(2-methyl-4-thiazolyl)ethynyl]-5-[2-(tributylstannyl)vinyl]pyridine (57), 3-[(2-methyl-4-thiazolyl)ethynyl]-5-vinylpyridine (59), bromoolefin (60), 5-[(2-methyl-4-thiazolyl)ethynyl]-1H-pyridin-2-one (61), methanesulfonic acid 5-[(2-methyl-4-thiazolyl)ethynyl]-2-pyridyl ester (62), 2-chloro-5-[(2-methyl-4-thiazolyl)ethynyl]pyridine (63), trifluoromethanesulfonic acid 5-[(2-methyl-4-thiazolyl)ethynyl]-2-pyridyl ester (64), 2-(4-fluorophenyl)-5-[(2-methyl-4-thiazolyl)ethynyl]pyridine (65), 3-ethynyl-5-[(2-methyl-4-thiazolyl)ethynyl]pyridine (66), 5-[(2-methyl-4-thiazolyl)ethynyl]-2-vinylpyridine (68), 3-iodo-2-methoxypyridine (69), 2-methoxy-3-[(2-methyl-4-thiazolyl)ethynyl]pyridine (70), bromoolefin (71), 3-[(2-methyl-4-thiazolyl)ethynyl]-1H-pyridin-2-one (72), methanesulfonic acid 3-[(2-methyl-4-thiazolyl)ethynyl]-2-pyridyl sster (73), 2-chloro-3-[(2-methyl-4-thiazolyl)ethynyl]pyridine (74), trifluoromethanesulfonic acid 5-[(2-methyl-4-thiazolyl)ethynyl]-2-pyridyl ester (75), 3-[(2-methyl-4-thiazolyl)ethynyl]-2-(trimethylsilylethynyl)pyridine (76), 2-ethynyl-3-[(2-methyl-4-thiazolyl)ethynyl]pyridine (77), 2-methyl-4-[2-(tributylstannyl)vinyl]thiazole (79), (E)-3-[2-(2-methyl-4-thiazolyl)vinyl]pyridine (80), 2-methylthiazole-4-carboxylic Acid 3-fluorophenylamide (81), 2-methylthiazole-4-carboxylic acid 3-pyridylamide (82), 2-methyloxazole-4-carboxylic acid methyl ester (84), 2-methyloxazole-4-carboxaldehyde (85), 4-(2,2-dibromovinyl)-2-methyloxazole (86), 2-methyl-4-(trimethylsilylethynyl)oxazole (88), 4-[(3-fluorophenyl)ethynyl]-2-methyloxazole (89), 3-[(2-methyl-4-oxazolyl)ethynyl]pyridine (90). Particular useful are compounds 19 and 59 that have a 490 and 230 times higher antagonistic potency, respectively, than MTEP.

5-[(2-methyl-1,3-thiazol-4-yl)ethynyl]-2,3′-bipyridine, a highly potent, orally active mGluR5 antagonist with anxiolytic activity (Roppe et al. 2004, Bioorg Med Chem Lett 14(15):3993-6) also can be used to practice the present invention.

Further, Roppe et al. (2004, J Med Chem 47(19):4645-8) and Tehrani et al. (2005, Bioorg Med Chem Lett 15(22):50614) described novel heteroarylazoles, 3-[substituted]-5-(5-pyridin-2-yl-2H-tetrazol-2-yl)benzonitriles, as mGluR5 antagonists having anxiolytic activity that can be used to practice the present invention. Preferred compounds for use in the present invention are 3-(5-pyridin-2-yl-2H-tetrazol-2-yl)benzonitrile (compound 47) and 3-fluoro-5-(5-pyridin-2-yl-2H-tetrazol-2-yl)benzonitrile (compound 48) (Roppe et al. (2004, J Med Chem 47(19):4645-8). 3-fluoro-5-(5-pyridin-2-yl-2H-tetrazol-2-yl)benzonitrile shows good rat pharmacokinetics, brain penetration, and in vivo receptor occupancy (Tehrani et al. 2005, Bioorg Med Chem Lett 15(22):5061-4). Structure-activity relationship (SAR) studies on 3-(5-pyridin-2-yl-2H-tetrazol-2-yl)benzonitrile led to the discovery of 2-(2-[3-(pyridine-3-yloxy)phenyl]-2H-tetrazol-5-yl)pyridine, a highly potent and selective mGluR5 receptor antagonist with good brain penetration and in vivo receptor occupancy in rat and cross-species oral bioavailability and useful to practice the present invention. In addition, SAR studies performed around 3-fluoro-5-(5-pyridin-2-yl-2H-tetrazol-2-yl)benzonitrile led to the synthesis of four-ring tetrazoles and to the discovery of 3-[3-fluoro-5-(5-pyridin-2-yl-2H-tetrazol-2-yl)phenyl]-4-methylpyridine, a highly potent, brain penetrant, azole-based mGluR5 antagonist (Poon et al., 2004, Bioorg Med Chem Lett 14(22):5477-80), which can also be used in the present invention.

Using high throughput screening (HTS), Hammerland et al. identified thiopyrimidine derivatives as potent mGluR5 antagonists (February 2006, Bioorg Med Chem Lett). Some of the compounds described by Hammerland show sub-micromolar activity.

Another preferred mGluR5 antagonist is fenobam [N-(3-chlorophenyl)-N′-(4,5-dihyfro-1-methyl-4-oxo-1H-imidazole-2-yl)urea], known to exert anxiolytic activity both in rodents and human. Fenobam has been reported to be a selective and potent mGluR5 antagonist acting at an allosteric modulatory site shared with MPEP (Porter et al., 2005, J Pharmacol Exp Ther 315(2):711-21). Additional functional analogues of fenobam are described by Wallberg et al. (2006, Bioorg Med Chem Lett 16(5):1142-5).

Other antagonists of mGluR5 and their preparation are also described in WO 01/66113, WO 01/32632, WO 01/14390, WO 01/08705, WO 01/05963, WO 01/02367, WO 01/02342, WO 01/02340, WO 00/20001, WO 00/73283, WO 00/69816, WO 00/63166, WO 00/26199, WO 00/26198, EP-A-0807621, WO 99/54280, WO 99/44639, WO 99/26927, WO 99/08678, WO 99/02497, WO 98/45270, WO 98/34907, WO 97/48399, WO 97/48400, WO 97/48409, WO 98/53812, WO 96/15100, WO 95/25110, WO 98/06724, WO 96/15099 WO 97/05109, WO 97/05137, U.S. Pat. Nos. 6,413,948, 6,288,046, 6,218,385, 6,071,965, 6,017,903, 6,054,444, 5,977,090, 5,968,915, 5,962,521, 5,672,592, 5,795,877, 5,863,536, 5,880,112, 5,902,817, all of which are hereby incorporated by reference.

For example, different classes of mGluR5 antagonists are described in WO 01/08705 (pp. 3-7), WO 99/44639 (pp. 3-11), and WO 98/34907 (pp. 3-20).

Another class of mGluR5 antagonists for use in the present invention is described in WO 01/02367 and WO 98/45270. Such compounds generally have the formula:

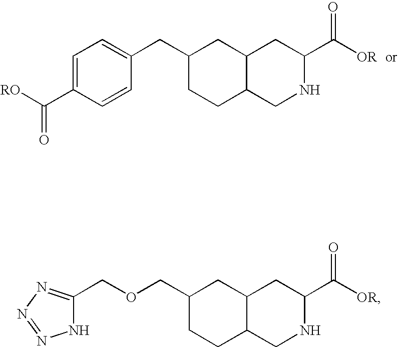

wherein R represents H or a hydrolyzable hydrocarbon moiety such as an alkyl, heteroalkyl, alkenyl, or aralkyl moiety.

In certain such embodiments, the isoquinoline system has the stereochemical array

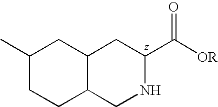

(wherein, as is known in the art, a dark spot on a carbon indicates hydrogen coming out of the page, and a pair of dashes indicates a hydrogen extending below the plane of the page), the enantiomer thereof, of a racemic mixture of the two.

Another class of antagonists, described in WO 01/66113, has the formula:

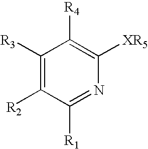

wherein
R₁ denotes hydrogen, lower alkyl, hydroxyl-lower alkyl, lower alkyl-amino, piperidino, carboxy, esterified carboxy, amidated carboxy, unsubstituted or lower alkyl-, lower alkoxy-, halo- and/or trifluoromethyl-substituted N-lower-alkyl-N-phenylcarbamoyl, lower alkoxy, halo-lower alkyl or halo-lower alkoxy;
R₂ denotes hydrogen, lower alkyl, carboxy, esterified carboxy, amidated carboxy, hydroxyl-lower alkyl, hydroxyl, lower alkoxy or lower alkanoyloxy, 4-(4-fluoro-benzoyl-piperidin-1-yl-carboxy, 4-t.butyloxycarbonyl-piperazin-1-yl-carboxy, 4-(4-azido-2-hydroxybenzoyl)-piperazin-1-yl-carboxy or 4-(4-azido-2-hydroxy-3-iodo-benzoyl)-piperazin-1-yl-carboxy;
R₃ represents hydrogen, lower alkyl, carboxy, lower alkoxy-carbonyl, lower alkyl-carbamoyl, hydroxy-lower alkyl, di-lower alkyl-aminomethyl, morpholinocarbonyl or 4-(4-fluoro-benzoyl)-piperazin-1-yl-carboxy;
R₄ represents hydrogen, lower alkyl, hydroxy, hydroxy-lower alkyl, amino-lower alkyl, lower alkylamino-lower alkyl, di-lower alkylamino-lower alkyl, unsubstituted or hydroxy-substituted lower alkyleneamino-lower alkyl, lower alkoxy, lower alkanoyloxy, amino-lower alkoxy, lower alkylamino-lower alkoxy, di-lower alkylamino-lower alkoxy, phthalimido-lower alkoxy, unsubstituted or hydroxy-or-2-oxo-imidazolidin-1-yl-substituted lower alkyleneamino-lower alkoxy, carboxy, esterified or amidated carboxy, carboxy-lower alkoxy or esterified carboxy-lower alkoxy; and
X represents an optionally halo-substituted lower alkenylene or alkynylene group bonded via vicinal saturated carbon atoms or an azo (—N═N—) group, and R₅ denotes an aromatic or heteroaromatic group which is unsubstituted or substituted by one or more substituents selected from lower alkyl, halo, halo-lower alkyl, halo-lower alkoxy, lower alkenyl, lower alkynyl, unsubstituted or lower alkyl-, lower alkoxy-, halo- and/or trifluoromethyl-substituted phenyl, unsubstituted or lower alkyl-, lower alkoxy-, halo and/or trifluoromethyl-substituted phenyl-lower alkynyl, hydroxy, hydroxy-lower alkyl, lower alkanoyloxy-lower alkyl, lower alkoxy, lower alkenyloxy, lower alkylenedioxy, lower alkanoyloxy, amino-, lower alkylamino-, lower alkanoylamino- or N-lower alkyl-N-lower alkanoylamino-lower alkoxy, unsubstituted or lower alkyl-, lower alkoxy-, halo- and/or trifluoromethyl-substituted phenoxy, unsubstituted or lower alkyl-, lower alkoxy-, halo and/or trifluoromethyl-substituted phenyl-lower alkoxy, acyl, carboxy, esterified carboxy, amidated carboxy, cyano, carboxy-lower alkylamino, esterified carboxy-lower alkylamino, amidated carboxy-lower alkylamino, phosphono-lower alkylamino-esterified phosphono-lower alkylamino, nitro, amino, lower alkylamino, di-lower alkylamino-acylamino, N-acyl-N-lower alkylamino, phenylamino, phenyl-lower alkylamino, cycloalkyl-lower alkylamino or heteroaryl-lower alkylamino each of which may be unsubstituted or lower alkyl-, lower alkoxy-, halo- and/or trifluoromethyl-substituted; their N-oxides and their pharmaceutically acceptable salts.

In certain such embodiments, as disclosed in WO 01/66113 and WO 00/20001, a mGluR5 antagonist has the formula:

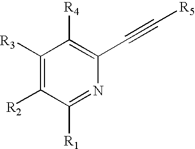

wherein
R₁ is hydrogen, (C₁₋₄)alkyl, (C₁₋₄)alkoxy, cyano, ethynyl or di(C₁₋₄)alkylamino;
R₂ is hydrogen, hydroxy, carboxy, (C₁₋₄) alkoxycarbonyl, di(C₁₋₄)alkylaminomethyl, 4-(4-fluoro-benzoyl)-piperidin-1-yl-carboxy, 4-t-butyloxycarbonyl-piperazin-1-yl-carboxy, 4-(4-azido-2-hydroxybenzoyl)-piperazin-1-yl-carboxy, or 4-(4-azido-2-hydroxy-3-iodo-benzoyl)-piperazin-1-yl-carboxy;
R₃ is hydrogen, (C₁₋₄)alkyl, carboxy, (C₁₋₄)alkoxycarbonyl, (C₁₋₄)alkylcarbamoyl, hydroxy(C₁₋₄)alkyl, di(C₁₋₄)alkylaminomethyl, morpholinocarbonyl or 4-(4-fluoro-benzoyl)-piperazin-1-yl-carboxy;
R₄ is hydrogen, hydroxyl, carboxy, C(₂₋₅)alkanoyloxy, (C₁₋₄)alkoxycarbonyl, amino (C₁₋₄)alkoxy, di(C₁₋₄)alkylamino(C₁₋₄)alkoxy, di(C₁₋₄)alkylamino(C₁₋₄)alkyl or hydroxy(C₁₋₄)alkyl; and
R₅ is a group of formula:

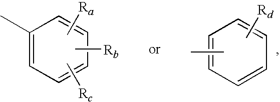

wherein
Rₐ and R_{b} independently are hydrogen, halogen, nitro, cyano, (C₁₋₄)alkyl, (C₁₋₄)alkoxy, trifluoromethyl, trifluoromethoxy or (C₂₋₅)alkynyl;
and R_{c} is hydrogen, fluorine, chlorine bromine, hydroxy-(C₁₋₄)alkyl, (C₂₋₅)alkanoyloxy, (C₁₋₄)alkoxy, or cyano; and
R_{d} is hydrogen, halogen or (C₁₋₄)alkyl;
in free form or in the form of pharmaceutically acceptable salts.

In certain other embodiments, as disclosed in WO 01/66113, mGluR5 antagonists have structures of the formula:

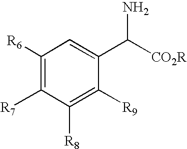

wherein
R₆ is hydrogen, hydroxy, or (C₁₋₆)alkoxy;
R₇ is hydrogen, carboxy, tetrazolyl, —SO₂H, —SO₃H, OSO₃H, —CONHOH, or —P(OH)OR′, —PO(OH)OR′, —OP(OH)OR′ or PO(OH)OR′ where R′ is hydrogen, (C₁₋₆)alkyl, (C₂₋₆)alkenyl, or aryl (C₁₋₆)aryl;
R₈ is hydrogen, hydroxy or (C₁₋₄)alkoxy; and
R₉ is fluoro, trifluoromethyl, nitro, (C₁₋₆)alkyl, (C₃₋₇)cycloalkyl, (C₂₋₆)alkenyl, (C₂₋₆)alkynyl, (C₁₋₆)alkylthio, heteroaryl, optionally substituted aryl, optionally substituted aryl (C₁₋₆)alkyl, optionally substituted aryl (C₂₋₆)alkenyl, optionally substituted aryl (C₂₋₆)alkynyl, optionally substituted aryloxy, optionally substituted (C₁₋₆)alkoxy, optionally substituted arythio, optionally substituted aryl (C₁₋₆)alkylthio, —CONR″; R′″, —NR″R′″, —OCONR″R′″ or —SONR″R′″, where R″; and R′″; are each hydrogen, (C₁₋₆)alkyl or aryl (C₁₋₆)alkyl, or R″ and R′″ together form a (C₃₋₇)alkylene ring;
or a salt or ester thereof.

Yet another class of mGluR5 antagonists useful to practice the invention is described in WO 00/63166. These compounds have the formula:

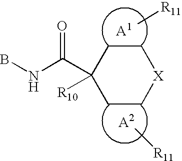

wherein
R₁₀ signifies hydrogen or lower alkyl;
R₁₁ signifies, independently for each occurrence, hydrogen, lower alkyl, lower alkoxy, halogen or trifluoromethyl;
X signifies O, S, or two hydrogen atoms not forming a bridge;
A¹/A² signify, independently from each other, phenyl or a 6-membered heterocycle containing 1 or 2 nitrogen atoms;
B is a group of formula:

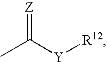

wherein
R¹² signifies lower alkyl, lower alkenyl, lower alkynyl, benzyl, lower alkyl-cycloalkyl, lower alkyl-cyano, lower alkyl-pyridinyl, lower alkyl-lower alkoxy-phenyl, lower alkyl-phenyl (optionally substituted by lower alkoxy), phenyl (optionally substituted by lower alkoxy), lower alkyl-thienyl, cycloalkyl, lower alkyl-trifluoromethyl, or lower alkyl-morpholinyl;
Y signifies —O—, —S— or bond;
Z signifies —O— or —S—;
or B is a 5-membered heterocyclic group of formulae

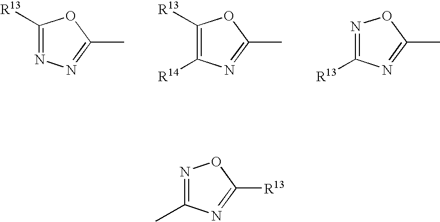

wherein
R¹³ and R¹⁴ independently signify hydrogen, lower alkyl, lower alkoxy, cyclohexyl, lower alkyl-cyclohexyl or trifluoromethyl, with the proviso that at least one of R¹³ or R¹⁴ is hydrogen;
as well as with their pharmaceutically acceptable salts.

Another class of mGluR5 antagonists useful to practice the invention is described in WO 01/14390. These compounds have the formula:

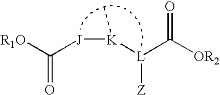

wherein
either J and K are taken together with one or more additional atoms independently selected from the group consisting of C, O, S, and N in chemically reasonable substitution patterns to form a 3-7 membered saturated or unsaturated heterocyclic or carbocyclic ring, and L is —CH,
or J, K, and L are taken together with one or more additional atoms independently selected from the group consisting of C, O, S, and N in chemically reasonable substitution patterns to form a 4-8 membered saturated or unsaturated, mono-, bi-, or tricyclic, hetero- or carbocyclic ring structure;
Z is a metal chelating group;
R₁ and R₂ are independently hydrogen, (C₁-C₉)alkyl, (C₂-C₈)alkenyl, (C₃-C₈) cycloalkyl, (C₅-C₇)cycloalkenyl, or Ar, wherein each said alkyl, alkenyl, cycloalkyl, cycloalkenyl, or Ar is independently unsubstituted or substituted with one or more substituent(s); and
Ar is a carbocyclic or heterocyclic moiety which is unsubstituted or substituted with one or more substituent(s);
or a pharmaceutically acceptable equivalent thereof.

Still another class of mGluR5 antagonists useful to practice the invention is described in U.S. Pat. No. 6,218,385. These compounds have the formula:

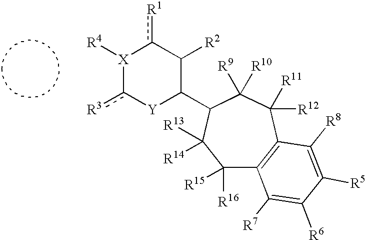

wherein
R¹ signifies hydrogen, hydroxy, lower alkyl, oxygen, halogen, or —OR, —O(C₃-C₆)cycloalkyl, —O(CHR)ₙ—(C₃-C₆)cycloalkyl, —O(CHR)ₙCN, —O(CHR)ₙCF₃, —O(CHR)(CHR)ₙNR₂, —O(CHR)(CHR)ₙOR, —O(CHR)ₙ-lower alkenyl, —OCF₃, —CF₂—R, —OCF₂-lower alkenyl, —OCHRF, —OCHF-lower alkenyl, —OCF₂CRF₂, —OCF₂Br, —O(CHR)ₙCF₂Br, —O(CHR)ₙ-phenyl, wherein the phenyl group may be optionally substituted independently from each other by one to three lower alkyl, lower alkoxy, halogen, nitro or cyano groups,
—O(CHR)(CHR)ₙ-morpholino, —O(CHR)(CHR)ₙ-pyrrolidino, —O(CHR)(CHR)ₙ-piperidino, —O(CHR)(CHR)ₙ-imidazolo, —O(CHR)(CHR)ₙ-triazolo, —O(CHR)ₙ-pyridino, —O(CHR)(CHR)ₙ—OSi-lower alkyl, —O(CHR)(CHR)ₙOS(O)₂-lower alkyl, —(CH₂)ₙCH═CF₂, —O(CHR)ₙ-2,2-dimethyl-[1,3]dioxolane, —O(CHR)ₙ—CHOR—CH₂OR, —O(CHR)ₙ—CHOR—(CHR)ₙ—CH₂OR or

### —SR or —S(CHR)ₙCOOR, or

—NR₂, —N(R)(CHR)(CHR)ₙOR, —N(R)(CHR)CF₃, —N(R)(CHR)(CHR)ₙ-morpholino, —N(R)(CHR)(CHR)ₙ-imidazolo, —N(R)(CHR)(CHR)ₙ-pyrrolidino, —N(R)(CHR)(CHR)ₙ-pyrrolidin-2-one, —N(R)(CHR)(CHR)ₙ-piperidino, —N(R)(CHR)(CHR)ₙ-triazolo, —N(R)(CHR)ₙ-pyridino, or
R¹ and R⁴ are interconnected to the groups —(CH₂)₃₋₅, —(CH₂)₂—N═, —CH═N—N═—, —CH═CH—N═, —NH—CH═CH— or —NR—CH₂—CH₂— and form together with any N or C atoms to which they are attached an additional ring;
n is 1-6;
R signifies hydrogen, lower alkyl or lower alkenyl, independently from each other, if more than one R is present;
R² signifies nitro or cyano;
R³ signifies hydrogen, lower alkyl, ═O, —S, —SR, —S(O)₂-lower alkyl, —C₃-C₆)cycloalkyl or piperazino, optionally substituted by lower alkyl, or
—CONR₂, —(CHR)ₙCONR₂, —CHR)ₙOR, —(CH₂)ₙ—CF₃, —CF₃, —CHR)ₙOC(O)CF₃, —(CHR)ₙCOOR, —(CHR)ₙSC₆H₅, wherein the phenyl group may be optionally substituted independently from each other by one to three lower alkyl, lower alkoxy, halogen, nitro or cyano groups,
—CHR)ₙ-1,3-dioxo-1,3-dihydro-isoindol, —(CHR)ₙ-tetrahydro-pyran-2-yloxy or —(CHR)ₙ—S-lower alkyl, or
—NR₂, —NRCO-lower alkyl, —NRCHO, —N(R)(CHR)ₙCN, —N(R)(CHR)ₙCF₃, —N(R)(CHR)(CHR)ₙ—OR, —N(R)C(O)(CHR)ₙO-lower alkyl, —NR(CHR)ₙ-lower alkyl, —NR(CHR)(CHR)ₙ—OR, —N(R)(CHR)(CHR)ₙ—O-phenyl, wherein the phenyl group may be optionally substituted independently from each other by one to three lower alkyl, lower alkoxy, halogen, nitro or cyano groups,
—N(R)(CHR)ₙ-lower alkenyl, —N(R)(CHR)(CHR)ₙ—O—(CHR)ₙOR, —N(R)(CHR)ₙC(O)O-lower alkyl, —N(R)(CHR)ₙC(O)NR-lower alkyl, —N(R)(CH₂)ₙ-2,2-dimethyl-[1,3]dioxolane, —N(R)(CHR)(CHR)ₙ-morpholino, —N(R)(CHR)ₙ-pyridino, —N(R)(CHR)(CHR)ₙ-piperidino, —N(R)(CHR)(CHR)ₙ-pyrrolidino, —N(R)(CHR)(CHR)ₙ—O-pyridino, —N(R)(CHR)(CHR)ₙ-imidazolo, —N(R)(CHR)ₙ—CR₂—(CHR)ₙ—OR, —N(R)(CHR)ₙ—CR₂—OR, —N(R)(CHR)ₙ—CHOR—CH₂OR, —N(R)(CHR)ₙ—CHOR—(CHR)ₙ—CH₂OR, or
—OR, —O(CHR)ₙCF₃, —OCF₃, —O(CHR)(CHR)ₙ—O-phenyl, wherein the phenyl group maybe optionally substituted independently from each other by one to three lower alkyl, lower alkoxy, halogen, nitro or cyano groups,
—O(CHR)(CHR)ₙ—O-lower alkyl, —O(CHR)ₙ-pyridino or —O(CHR)(CHR)ₙ-morpholino;
or R³ and R⁴ are interconnected to the groups —(CH₂)₃₋₅—, —(CH₂)₂—N═, —CH═N—N═—, —CH═CH—N═, —NH—CH═CH — or —NR—CH₂—CH₂— and form together with any N or C atoms to which they are attached an additional ring; and
R⁴ signifies hydrogen, lower alkyl, lower alkenyl or nitro, or —OR, —OCF₃, —OCF₂—R, —OCF2-lower alkenyl, —OCHRF, —OCHF-lower alkenyl, —O(CHR)ₙCF₃, or —(CHR)ₙCHRF, —(CHR)ₙCF₂R, —(CHR)ₙCF₃, —(C₃-C₆)cycloalkyl, —CHR)ₙ(C₃-C₆)cycloalkyl, —(CHR)ₙCN, —(CHR)ₙ-phenyl, wherein the phenyl group may be optionally substituted independently from each other by one, to three lower alkyl, lower alkoxy, halogen, nitro or cyano groups,
—(CHR)(CHR)ₙOR, —(CHR)ₙCHORCH₂OR, —(CHR)(CHR)ₙNR₂, —(CHR)ₙCOOR, —(CHR)(CHR)ₙOSi-lower alkyl, —(CHR)(CHR)ₙ—OS(O)₂-lower alkyl, —(CH₂)ₙ—CH═CF₂, —CF₃, —CF₂—R, —CF₂-lower alkenyl, —CHRF, —CHF-lower alkenyl, —(CHR)ₙ-2,2-dimethyl-[1,3]dioxolane, —(CH₂)ₙ-2-oxo-azepan-1-yl, —(CHR)(CHR)ₙ-morpholino, —(CHR)ₙ-pyridino, —(CHR)(CHR)ₙ-imidazolo, —(CHR)(CHR)ₙ-triazolo, —(CHR)(CHR)ₙ-pyrrolidino, optionally substituted by —(CH₂)ₙOH, —(CHR)(CHR)ₙ-3-hydroxy-pyrrolidino or —(CHR)(CHR)ₙ-piperidino, or
—NR₂, —N(R)(CHR)ₙ-pyridino, —N(R)C(O)O-lower alkyl, —N(CH₂CF₃)C(O)O-lower alkyl, —N[C(O)O-lower alkyl]₂, —NR—NR—C(O)O-lower alkyl or —N(R)(CHR)ₙCF₃, —NRCF₃, —NRCF₂—R, —NRCF₂-lower alkenyl, —NRCHRF, —NRCHP-lower alkenyl;
or is absent if X is —N═ or ═N—;
R⁵, R⁶ signify hydrogen, lower alkyl, lower alkoxy, amino, nitro, —SO₂NH₂ or halogen; or
R⁵ and R⁶ are interconnected to the group —O—CH₂—O— and form together with the C atoms to which they are attached an additional 5-membered ring;
R⁷, R⁸ signify hydrogen, lower alkyl, lower alkoxy, amino, nitro or halogen;
R⁹, R¹⁰ signify hydrogen or lower alkyl;
R¹¹, R¹² signify hydrogen, lower alkyl, hydroxy, lower alkoxy, lower alkoxycarbonyloxy or lower alkanoyloxy;
R¹³, R¹⁴ signify hydrogen, tritium or lower alkyl;
R¹⁵, R¹⁶ signify hydrogen, tritium, lower alkyl, hydroxy, lower alkoxy or are together an oxo group; or
X signifies —N═, ═N—, —N<, >C═ or ═C<;
Y signifies —N═, ═N—, —NH—, —CH═ or ═CH—; and
the dotted line may be a bond when R¹, R³ or R⁴ represent a bivalent atom, as well as with the pharmaceutically acceptable salts of each compound of the above formula and the racemic and optically active forms of each compound of the above formula.

Yet other classes of mGluR5 antagonists useful to practice the invention are described in WO 01/02342 and WO 01/02340. These compounds have the formulas, respectively:

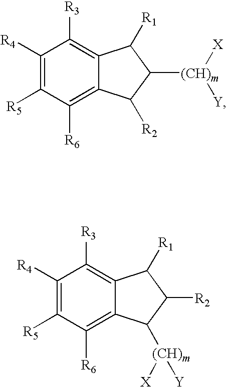

stereoisomers thereof, or pharmaceutically acceptable salts or hydrates thereof, wherein:
R₁ and R₂ are either:

### 1) H; or

2) an acidic group selected from the group consisting of carboxy, phosphono, phosphino, sulfono, suloino, borono, tetrazol, isoxazol, —(CH₂)ₙ-carboxy, —(CH₂)ₙ-phosphono, —(CH₂)ₙ-phosphino, —(CH₂)ₙ-sulfono, —(CH₂)ₙ-sulfino, —(CH₂)ₙ-borono, —(CH₂)ₙ-tetrazol, and —(CH₂)ₙ-isoxazol, where n=1, 2, 3, 4, 5, or 6;
X is an acidic group selected from the group consisting of carboxy, phosphono, phosphino, sulfono, sulfino, borono, tetrazol, and isoxazol;
Y is a basic group selected from the group consisting of 1° amino, 2° amino, 3° amino, quaternary ammonium salts, aliphatic 1° amino, aliphatic 2° amino, aliphatic 3° amino, aliphatic quaternary ammonium salts, aromatic 1° amino, aromatic 2° amino, aromatic 3° amino, aromatic quaternary ammonium salts, imidazol, guanidino, boronoamino, allyl, urea, and thiourea;
m is 0, 1; and
R₃, R₄, R₅, R₆ are independently H, nitro, amino; halogen, tritium, trifluoromethyl, trifluoroacetyl, sulfo, carboxy, carbamoyl, sulfamoyl or acceptable esters thereof;
or a salt thereof with a pharmaceutically acceptable acid or base.

Further classes of mGluR5 antagonists are described in WO 00/73283 and WO 99/26927. These compounds have the formula: R-[Linker]-Ar, wherein R is an optionally substituted straight or branched chain alkyl, arylalkyl, cycloalkyl, or alkylcycloalkyl group preferably containing 5-12 carbon atoms; Ar is an optionally substituted aromatic, heteroaromatic, arylalkyl, or heteroaralkyl moiety containing up to 10 carbon atoms and up to 4 heteroatoms; and [linker] is —(CH₂)ₙ—, where n is 2-6, and wherein up to 4 CH₂ groups may independently be substituted with groups selected from the group consisting of C₁-C₃ alkyl, CHOH, CO, O, S, SO, SO₂, N, NH, and NO. Two heteroatoms in the [linker] may not be adjacent except when those atoms are both N (as in —N═N— of —NH—NH—) or are N and S as in a sulfonamide. Two adjacent CH₂ groups in [linker] also may be replaced by a substituted or unsubstituted alkene or alkyne group. Pharmaceutically acceptable salts of the compounds also are provided.

Another class of mGluR5 antagonists useful to practice the invention is described in WO 00/69816. These compounds have the formula:

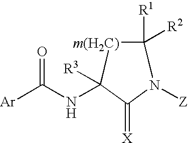

wherein
m is 0, 1 or 2;

### X is O, S, NH, or NOH;

R¹ and R² are each independently H, CN, COOR, CONHR, (C₁-C₆)alkyl, tetrazole, or R and R² together represent “═O”;
R is H or (C₁-C₆)alkyl;
R³ is (C₁-C₆)alkyl, (C₂-C₆)alkenyl, (C₃-C₆)cycloalkyl, —CH₂OH, —CH₂O-alkyl, —COOH;
Ar is an unsubstituted or substituted aromatic or heteroaromatic group;
Z represents a group of the formulae:

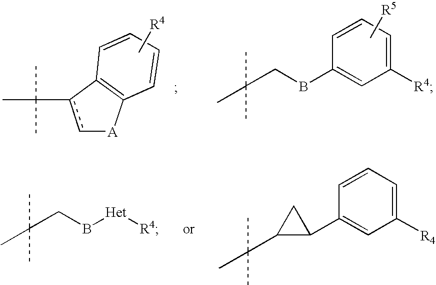

wherein
R⁴ and R⁵ are each independently H, halogen, (C₁-C₆)alkoxy, —OAr, (C₁-C₆)alkyl, —CF₃, —COOR, —CONHR, —CN, —OH, —COR, —S(C₁-C₆)alkyl), —SO₂((C₁-C₆)alkyl);
A is CH₂, O, NH, NR, S, SO, SO₂, CH₂—CH₂, CH₂O, CHOH, C(O); wherein R is as defined above;
B is CHR, CR₂, (C₁-C₆)alkyl, C(O), —CHOH, —CH₂—O, —CH═CH, CH₂—C(O), CH₂—S, CH₂—S(O), CH₂—SO₂; —CHCO₂R; or —CH—NR₂, wherein R is as defined above;
Het is a heterocycle such as furan, thiophene, or pyridine;
or a pharmaceutically acceptable salt thereof

Another class of mGluR5 antagonists useful to practice the invention is described in WO 99/54280. These compounds have the formula:

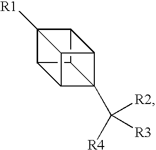

wherein
R1 can be an acidic group selected from the group consisting of carboxyl, phosphono, phosphino, sulfono, sulfino, borono, tetrazol, isoxazol, —CH₂-carboxyl, —CH₂-phosphono, —CH₂-phosphino, CH₂-sulfono, —CH₂-sulfino, —CH₂-borono, —CH₂-tetrazol, —CH₂-isoxazol and higher homologues thereof;
R2 can be a basic group selected from the group consisting of 1° amino, 2° amino, 3° amino, quaternary ammonium salts, aliphatic 1° amino, aliphatic 2° amino, aliphatic 3° amino, aliphatic quaternary ammonium salts, aromatic 1° amino, aromatic 2° amino, aromatic 3° amino, aromatic quaternary ammonium salts, imidazol, guanidino, boronoamino, allyl, urea, and thiourea;
R3 can be H, aliphatic, aromatic or heterocyclic;
R4 can be an acidic group selected from the group consisting of carboxyl, phosphono, phosphino, sulfono, sulfino, borono, tetrazol, and isoxazol; stereoisomers thereof; and pharmaceutically acceptable salts thereof.

Yet another class of mGluR5 antagonists useful to practice the invention is described in WO 99/08678. These compounds have the formula:

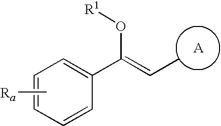

wherein
R signifies halogen or lower alkyl; n signifies 0-3;
R¹ signifies lower alkyl; cycloalkyl; benzyl optionally substituted by hydroxy, halogen, lower alkoxy or lower alkyl; benzoyl optionally substituted by amino, lower alkylamino or di-lower alkylamino; acetyl or cycloalkyl-carbonyl; and

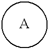

signifies an aromatic 5-membered residue which is bonded via a N-atom and which contains further 1-3 N atoms in addition to the linking N atom, as well as their pharmaceutically acceptable salts.

Preferred mGluR5 antagonists are those that provide an increase of expression of a neurotrophic factor above an expression level of the neurotrophic factor achieved by administration of an AMPAKINE® alone. The expression level of a neurotrophic factor by an AMPAKINE® alone may be predetermined prior to administration of an mGluR5 antagonist. Preferably, the increase of a neurotrophic factor expression upon administration of a mGluR5 antagonist is at least about 20%, and more preferably at least about 30%, at least about 40%, at least about 50%, at least about 60%, at least about 70%, at least about 80%, at least about 90%, at least about 100%, more even preferably at about 150-200% and more at a concentration of the antagonist, for example, of 1 μg/ml, 10 μg/ml, 100 μg/ml, 500 μg/ml, 1 mg/ml, 10 mg/ml or 30 mg/ml.

The percentage increase of neurotrophic factor expression can be determined as described herein, i.e., by determining expression level of the neurotrophic factor mRNA or of the neurotrophic factor polypeptide.

C. Identifying and Testing AMPAKINES® and Group 1 Metabotropic Glutamate Receptor Antagonists

Methods for identifying and assaying compounds, AMPAKINES® and mGluR5 antagonists, other than those disclosed herein and useful to practice the present invention are routine. They involve a variety of accepted tests to determine whether a given candidate compound is an allosteric upmodulator (such as AMPAKINES® described herein), or a mGluR5 antagonist.

1. Assays for AMPAKINES®

Because any positive AMPA receptor modulator can be used for practicing the methods of the present invention, in addition to the compounds and compositions described herein, additional useful positive AMPA receptor modulators can be determined by the skilled artisan. A variety of such routine, well-known methods can be used and are described in the scientific and patent literature. They include in vitro and in vivo assays for the identification of additional positive AMPA receptor modulators as described herein and for example, in U.S. Pat. Nos. 5,747,492, 5,773,434, 5,852,008, 5,891,876, 6,030,968, 6,083,947, 6,166,008, and 6,274,600, which are incorporated in their entirety by reference.

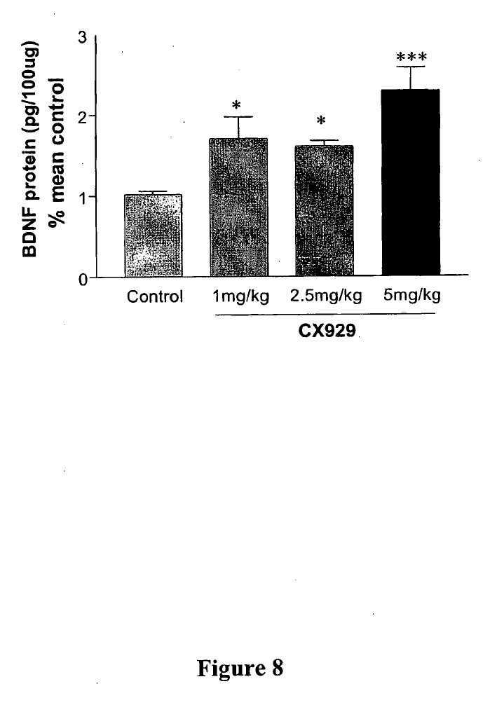

AMPAKINES® described herein and novel AMPAKINES® can be screened for activity in vitro and in vivo. For in vitro assays, this invention provides cell-based assays, as described herein (e.g., see Examples 2-7). For in vivo assays, this invention provides mouse/rat assays as described herein (e.g., see FIG. 8 for measuring in vivo BDNF protein levels following treatment with CX929 using ELISA).

A primary assay for testing the activity of an AMPAKINE® is measurement of enlargement of the excitatory postsynaptic potential (EPSP) in in vitro brain slices, such as rat hippocampal brain slices. In this assay, slices of hippocampus from a mammal such as rat are prepared and maintained in an interface chamber using conventional methods. Field EPSPs are recorded in the stratum radiatum of region CA1b and elicited by single stimulation pulses delivered once per 20 seconds to a bipolar electrode positioned in the Schaffer-commissural projections (see Granger et al., 1993, Synapse 15:326-329; Staubli et al., 1994a, Proc. Natl. Acad. Sci. USA 91:777-781; Staubli et al., 1994b, Proc. Natl. Acad. Sci. USA 91:11158-11162; Arai et al., 1994, Brain Res 638:343-346; Arai et al., 1996a, Neuroscience 75:573-585, and Arai et al., 1996, J Pharm Exp Ther 278:627-638). This assay can also be used to determine if a mGluR antagonist, and in particular an mGluR5 antagonist, potentiates the effect of an AMPAKINE®, as described herein.

Compounds of the present invention, such as AMPAKINES® and mGluR5 antagonists may also comprise a label. In one embodiment of the present invention, a compound contains unnatural proportions of atomic isotopes at one or more of the atoms that constitute such compound. For example, a compound may be radiolabeled with radioactive isotopes, such as for example tritium (³H) or carbon-14 (¹⁴C). All isotopic variations of the compounds of the present invention, whether radioactive or not, are intended to be encompassed within the scope of the present invention.

2. Assays for mGluR5 Antagonists

Because any mGluR5 antagonist can be used for practicing the methods of the present invention, in addition to the compounds and compositions described herein, additional useful mGluR5 antagonists can be determined by the skilled artisan. A variety of such routine, well-known methods can be used and are described in the scientific and patent literature. They include in vitro and in vivo assays for the identification of additional mGluR5 antagonists as described herein and for example, in WO 01/66113, WO 01/32632, WO 01/14390, WO 01/08705, WO 01/05963, WO 01/02367, WO 01/02342, WO 01/02340, WO 00/20001, WO 00/73283, WO 00/69816, WO 00/63166, WO 00/26199, WO 00/26198, EP-A-0807621, WO 99/54280, WO 99/44639, WO 99/26927, WO 99/08678, WO 99/02497, WO 98/45270, WO 98/34907, WO 97/48399, WO 97/48400, WO 97/48409, WO 98/53812, WO 96/15100, WO 95/25110, WO 98/06724, WO 96/15099 WO 97/05109, WO 97/05137, U.S. Pat. Nos. 6,413,948, 6,288,046, 6,218,385, 6,071,965, 6,017,903, 6,054,444, 5,977,090, 5,968,915, 5,962,521, 5,672,592, 5,795,877, 5,863,536, 5,880,112, 5,902,817, all of which are hereby incorporated by reference.

Methods for identifying mGluR antagonists, and in particular mGluR5 antagonists, which may be used in a method described herein, are known in the art. One example of an assay for determining the activity of a test compound as an antagonist of mGluR5 comprises expressing mGluR5 in CHO cells which have been transformed with cDNAs encoding the mGluR5 receptor protein (Daggett et al., 1995, Neuropharmacology 34:871-86). The mGluR5 is then activated by the addition of quisqualate and/or glutamate and can be assessed by, for example the measurement of: (i) phosphoinositol hydrolysis (Litschig et al., 1999, Mol Pharmacol 55:453-61); (ii) accumulation of [³H] cytidinephosphate-diacylglycerol (Cavanni et al., 1999, Neuropharmacology 38:A10); or fluorescent detection of calcium influx into cells Kawabata et al., 1996, Nature 383:89-92; Nakahara et al., 1997, J Neurochemistry 69:1467-74). This assay is amenable to high throughput screening.

Further, GluR5 receptor antagonists may also be identified by radiolabeled ligand binding studies at the cloned and expressed human GluR5 receptor (Korczak et al., 1994, Recept Channels 3:41-49), by whole cell voltage clamp electro-physiological recordings of functional activity at the human GluR5 receptor (Korczak et al., 1994, Recept Channels 3:41-49) and by whole cell voltage clamp electro-physiological recordings of currents in acutely isolated rat dorsal-root ganglion neurons (Bleakman et al., 1996, Mol Pharmacol 49:581-585).

### III. Synergistic Effects of Positive AMPA Receptor Modulators and Group 1 Metabotropic Glutamate Receptor Antagonists

The compounds of the present invention find use in a variety of ways. Methods of present invention used to treat a condition or disorder are based on the surprising discovery that synaptic responses mediated by AMPA receptors are increased by co-administration of an AMPAKINE® and a mGluR5 antagonist (compared to administration of the AMPAKINE® alone) and further that co-administration of an AMPAKINE® and a mGluR5 antagonist leads to increased expression of neurotrophic factors (compared to administration of the AMPAKINE® alone).

Downregulation of or reduced expression of a neurotrophic factor, for example, reduced BDNF expression, is indicative of and can be correlated with various conditions or diseases described. Thus, a BDNF polypeptide or a BDNF polynucleotide can be used as a biomarker in the diagnosis of a condition or disease. In one preferred embodiment of the present invention, the amount of BDNF in a biological sample is determined. Typically, the amount of BDNF in a biological sample provided from a normal, healthy subject is correlated with the amount of BDNF in a biological sample provided from a subject having a condition or disease as described herein or being suspected of having such a condition or disease. The amount of BDNF detected in the biological sample from a subject having a condition or disease or from the subject suspected of having a condition or disease may be specific for a given condition or disease.

Recently it was shown that AMPAKINES®, such as CX 614 (2H,3H,6aH-pyrrolidino[2″,1″-3′,2′]1,3-oxazino[6′,5′-5,4]benzo[e]1,4-dioxan-10-one) or CX546 markedly and reversibly increased brain-derived neurotrophic factor (e.g., BDNF and NGF) mRNA and protein levels in cultured rat entorhinal/hippocampal slices in a dose-dependent manner (Lauterborn et al., 2000, J Neurosci 20(1):8-21). These results suggested that neuroprotective treatments based on elevated levels of endogenous BDNF and NGF are feasible. Further studies using CX614 and LY392098 showed that these compounds rapidly increased BDNF expression, but with time, mRNA levels fell despite the continued presence of the drug (Lauterborn et al., 2003, J Pharmacol Exp Ther 307(1):297-305; Legutko et al., 2001, Neuropharmacology 40:1019-1027). Thus, although effective, AMPAKINES® may not sustain a high level expression of a neurotrophic factor expression, such as BDNF. To applicants' knowledge, no means to further sustain or increase neurotrophic factor expression above the level induced by AMPAKINES® alone were reported prior to this invention.

A. Method for Increasing the Level Of A Neurotrophic Factor

The present invention discloses the surprising finding that a mGluR5 antagonist, which typically has no substantial or no effect on the expression of a neurotrophic factor, can potentiate the expression of a neurotrophic factor above an expression level obtained by administration of an AMPA-receptor allosteric upmodulator (AMPAKINE®) alone. In one embodiment of the present invention, the mGluR5 antagonist potentiates the expression of a neurotrophic factor mRNA. In another preferred embodiment, the mGluR5 antagonist potentiates the expression of a neurotrophic factor protein.

In one aspect of the present invention, administration of a mGLuR5 antagonist, such as MPEP, potentiates the expression of a neurotrophic factor above an expression level obtained with an AMPA-receptor allosteric upmodulator (AMPAKINE®) alone. In another embodiment of the present invention, administration of more than one mGluR5 antagonist, for example, MPEP and SIB 1893, potentiate the expression of a neurotrophic factor.

In one aspect of the present invention, the method of increasing the expression of a neurotrophic factor is performed in vivo. The method can also be performed in vitro, for example, in cell culture or in hippocampal slices as described herein.

1. Detection of Neurotrophic Factor mRNA

In a preferred embodiment the present invention provides a method for increasing the level of a neurotrophic factor mRNA in the brain of a mammal afflicted with a pathology, the method comprising the steps of the (a) administering to the mammal an amount of an AMPA-receptor allosteric upmodulator effective to increase the expression of the neurotrophic factor mRNA in the brain of the mammal; and (b) administering to the mammal an amount of a group 1 metabotropic glutamate receptor antagonist, preferably a mGluR5 antagonist, effective to increase the expression of the neurotrophic factor mRNA in the brain of the mammal above the level exhibited by step (a) alone; wherein the level of the neurotrophic factor mRNA in the brain of a mammal is increased.

A preferred neurotrophic factor mRNA is a BDNF mRNA. Thus, expression levels of a neurotrophic factor mRNA, preferably a BDNF mRNA, may be determined. Detecting a reduced expression level of the BDNF mRNA relative to normal indicates the presence of a condition or disease in the subject. In one embodiment, the step of determining the level of the BDNF mRNA comprises an amplification reaction. Methods of evaluating RNA expression of a particular gene are well known to those of skill in the art, and include, inter alia, hybridization and amplification based assays.

a) Direct Hybridization-Based Assays

Methods of detecting and/or quantifying the level of a gene transcript (mRNA or cDNA made therefrom) using nucleic acid hybridization techniques are known to those of skill in the art. For example, one method for evaluating the presence, absence, or quantity of BDNF polynucleotides involves a Northern blot. Gene expression levels can also be analyzed by techniques known in the art, e.g., dot blotting, in situ hybridization, RNase protection, probing DNA microchip arrays, and the like. In situ hybridization and quantification of in situ hybridization, are described herein and in the art, for example, to determine BDNF and NGF mRNA expression (Lauterborn et al., 2000, J Neurosci 20(1):8-21; Lauterborn et al., 2003, J Pharmacol Exp Ther 307(1):297-305).

b) Amplification-Based Assays

In another embodiment, amplification-based assays are used to measure the expression level of a neurotrophic factor gene, preferably the expression level of a BDNF gene. In such an assay, the neurotrophic factor nucleic acid sequences act as a template in an amplification reaction (e.g., Polymerase Chain Reaction, or PCR). In a quantitative amplification, the amount of amplification product will be proportional to the amount of template in the original sample. Comparison to appropriate controls provides a measure of the level of neurotrophic factor mRNA in the sample. Methods of quantitative amplification are well known to those of skill in the art. Detailed protocols for quantitative PCR are provided, e.g., in Innis et al. (1990) PCR Protocols, A Guide to Methods and Applications, Academic Press, Inc. N.Y.). The known nucleic acid sequences for neurotrophic factor, such as BDNF (see, e.g., GenBank Accession Nos. herein) is sufficient to enable one of skill to routinely select primers to amplify any portion of the gene.

In one embodiment, a TaqMan based assay is used to quantify the neurotrophic factor polynucleotides. TaqMan based assays use a fluorogenic oligonucleotide probe that contains a 5′ fluorescent dye and a 3′ quenching agent. The probe hybridizes to a PCR product, but cannot itself be extended due to a blocking agent at the 3′ end. When the PCR product is amplified in subsequent cycles, the 5′ nuclease activity of the polymerase, e.g., AmpliTaq, results in the cleavage of the TaqMan probe. This cleavage separates the 5′ fluorescent dye and the 3′ quenching agent, thereby resulting in an increase in fluorescence as a function of amplification (see, for example, Heid et al., 1996, Genome Res 6(10):986-94; Morris et al., 1996, J Clin Microbiol 34(12):2933-6).

Other suitable amplification methods include, but are not limited to, ligase chain reaction (LCR) (see, Wu and Wallace, 1989, Genomics 4:560; Landegren et al., 1988, Science 241:1077; and Barringer et al., 1990, Gene 89:117), transcription amplification (Kwoh et al., 1989, Proc Natl Acad Sci USA 86:1173), self-sustained sequence replication (Guatelli et al., 1990, Proc Nat Acad Sci USA 87: 1874), dot PCR, and linker adapter PCR, etc.

2. Detection of Neurotrophic Factor Protein

In another preferred embodiment the present invention provides a method for increasing the level of a neurotrophic factor protein in the brain of a mammal afflicted with a pathology, the method comprising the steps of the (a) administering to the mammal an amount of an AMPA-receptor allosteric upmodulator effective to increase the expression of the neurotrophic factor protein in the brain of the mammal; and (b) administering to the mammal an amount of a group 1 metabotropic glutamate receptor antagonist, preferably a mGluR5 antagonist, effective to increase the expression of the neurotrophic factor protein in the brain of the mammal above the level exhibited by step (a) alone; wherein the level of the neurotrophic factor protein in the brain of a mammal is increased.

Expression of neurotrophic factors or receptors thereof can be detected by any of a number of methods known to those of skill in the art. Thus, expression can be assayed using antibodies specific to neurotrophic factors or neurotrophic factor receptors as measured or determined by standard antibody-antigen or ligand-receptor assays, for example, competitive assays, saturation assays, or standard immunoassays such as ELISA or RIA.

A preferred neurotrophic factor protein is a BDNF protein. Thus, expression level of a BDNF protein may be determined. Expression of a neurotrophic factor protein, preferably a BDNF protein, can be detected by several methods, including, but not limited to, affinity capture, mass spectrometry, traditional immunoassays directed to BDNF, PAGE, Western Blotting, or HPLC as further described herein or as known by one of skill in the art. Immunoassays and immunocytochemistry, are described herein and in the art, for example, to determine BDNF protein expression (Lauterborn et al., 2000, J Neurosci 20(1):8-21; Lauterborn et al., 2003, J Pharmacol Exp Ther 307(1):297-305).

Detection paradigms that can be employed to this end include optical methods, electrochemical methods (voltametry and amperometry techniques), atomic force microscopy, and radio frequency methods, e.g., multipolar resonance spectroscopy. Illustrative of optical methods, in addition to microscopy, both confocal and non-confocal, are detection of fluorescence, luminescence, chemiluminescence, absorbance, reflectance, transmittance, and birefringence or refractive index (e.g., surface plasmon resonance, ellipsometry, a resonant mirror method, a grating coupler waveguide method or interferometry).

B. Method for Increasing the Level of a Neurotrophic Factor Receptor

In an additional aspect, the present invention is directed to a method for increasing the expression of a neurotrophic factor receptor in a mammalian brain in a mammal in need of an increased expression of the neurotrophic factor receptor. In a preferred embodiment of the present invention this method comprises the steps of (a) administering to the mammal an amount of an AMPA-receptor allosteric upmodulator effective to increase the expression of the neurotrophic factor in the brain of the mammal; and (b) administering to the mammal an amount of a group 1 metabotropic glutamate receptor antagonist effective to increase the expression of the neurotrophic factor in the brain of the mammal above the level exhibited by step (a) alone; wherein the expression of the neurotrophic factor receptor is increased.

In one embodiment, the mammal is afflicted with a pathology which produces neurodegeneration without significant loss of memory or learning.

In yet another embodiment, the neurotrophic factor receptor is the TrkB receptor.

Determining expression levels of a neurotrophic factor receptor can be performed similarly to the methods for determining expression levels of a neurotrophic factor described above.

C. Method for Increasing TrkB Receptor Phosphorylation or Signaling

BDNF binds to TrkB receptor and stimulates TrkB receptor autophosphorylation and signaling. Thus, in an additional aspect, the present invention is directed to a method for increasing TrkB receptor phosphorylation or signaling in a mammalian brain in a mammal in need of an increased expression of the neurotrophic factor receptor. In a preferred embodiment of the present invention this method comprises the steps of (a) administering to the mammal an amount of an AMPA-receptor allosteric upmodulator effective to increase the expression of the neurotrophic factor in the brain of the mammal; and (b) administering to the mammal an amount of a group 1 metabotropic glutamate receptor antagonist effective to increase the expression of the neurotrophic factor in the brain of the mammal above the level exhibited by step (a) alone; wherein TrkB receptor phosphorylation or signaling is increased.

An increase of TrkB receptor phosphorylation or signaling is measured by comparing TrkB receptor phosphorylation or signaling in a cell or a mammalian brain treated with an AMPA-receptor allosteric upmodulator and a group 1 metabotropic glutamate receptor antagonist to TrkB receptor phosphorylation or signaling in an untreated cell or in an untreated mammalian brain. Assays for measuring phosphorylation of receptors, and in particular phosphorylation of a TrkB receptor, are well known in the art (e.g., Ibanez et al., 1993, EMBO J. 12(6):2281-93).

D. Method for Treating a Neurodegenerative Pathology

The present invention provides for an increase in the levels of neurotrophic factors and their receptors in mammalian brains. Thus, the methods disclosed herein provide therapeutic benefit to mammals afflicted with, or diagnosed as having, a neurodegenerative pathology characterized at least in part by a lower expression of a neurotrophic factor, when compared to the expression of the neurotrophic factor in a healthy mammal. In particular, the present invention is beneficial in the treatment of neurodegenerative pathologies including, but not limited to those, arising from a disease state and/or having an excitotoxic/ischemic mechanism.

Neurodegenerative pathologies that would benefit from this invention include conditions (diseases and insults) leading to neuronal cell death and/or sub-lethal neuronal pathology including, for example: (i) diseases of central motor systems including degenerative conditions affecting the basal ganglia (Huntington's disease, Wilson's disease, Striatonigral degeneration, corticobasal ganglionic degeneration), Tourettes syndrome, Parkinson's disease, progressive supranuclear palsy, progressive bulbar palsy, familial spastic paraplegia, spinomuscular atrophy, ALS and variants thereof, dentatorubral atrophy, olivo-pontocerebellar atrophy, paraneoplastic cerebellar degeneration; (ii) diseases affecting sensory neurons such as Friedreich's ataxia, diabetes, peripheral neuropathy, retinal neuronal degeneration; (iii) diseases of limbic and cortical systems such as cerebral amyloidosis, Pick's atrophy, Retts syndrome; (iv) neurodegenerative pathologies not causing significant loss of memory or learning involving multiple neuronal systems and/or brainstem including Leigh's disease, diffuse Lewy body disease, epilepsy, Multiple system atrophy, Guillain-Barre syndrome, lysosomal storage disorders such as lipofuscinosis, degenerative stages of Down's syndrome, Alper's disease, vertigo as result of CNS degeneration; (v) pathologies arising with aging and chronic alcohol or drug abuse including, for example, with alcoholism the degeneration of neurons in locus coeruleus and cerebellum; with aging degeneration of cerebellar neurons and cortical neurons leading to cognitive and motor impairments; and with chronic amphetamine abuse degeneration of basal ganglia neurons leading to motor impairments; (vi) pathological changes resulting from focal trauma such as stroke, focal ischemia, vascular insufficiency, hypoxic-ischemic encephalopathy, hyperglycemia, hypoglycemia or direct trauma; and (vii) pathologies arising as a negative side-effect of therapeutic drugs and treatments (e.g., degeneration of cingulate and entorhinal cortex neurons in response to anticonvulsant doses of antagonists of the NMDA class of glutamate receptor).

Mammals displaying clinical manifestations of a neurodegenerative pathology and in need of the therapeutic benefit derived from an increase in neurotrophic factors or neurotrophic factor receptors can be administered allosteric modulators and a mGluR5 antagonist according to the methods provided herein. Thus, in a preferred aspect, the present invention provides a method for increasing the level of a neurotrophic factor in a brain of a mammal afflicted with a neurodegenerative pathology. In a preferred embodiment, this method comprises the steps (a) administering to the mammal an amount of an AMPA-receptor allosteric upmodulator effective to increase the expression of the neurotrophic factor in the brain of the mammal; and (b) administering to the mammal an amount of a group 1 metabotropic glutamate receptor antagonist effective to increase the expression of the neurotrophic factor in the brain of the mammal above the level exhibited by step (a) alone; whereby the level of a neurotrophic factor in the brain of the mammal afflicted with the neurodegenerative pathology is increased and wherein the neurodegenerative pathology is improved.

Methods of evaluating the effects of the invention can be used which may be invasive or noninvasive. For example, therapeutic benefit includes any of a number of subjective or objective factors indicating a response of the condition being treated. This includes measures of increased neuronal survival or more normal function of surviving brain areas. For instance, some subjective symptoms of neurodegenerative disorders include pain, change in sensation including decreased sensation, muscle weakness, coordination problems, imbalance, neurasthenia, malaise, decreased reaction times, tremors, confusion, uncontrollable movement, lack of affect, obsessive/compulsive behavior, aphasia, agnosia, and visual neglect. Frequently objective signs, or signs observable by the physician or the health care provider, overlap with subjective signs. Examples include the physician's observation of signs such as decreased reaction time, muscle faciculations, tremors, rigidity, spasticity, muscle weakness, poor coordination, disorientation, dysphasia, dysarthria, and imbalance. Additionally, objective signs can include laboratory parameters such as the assessment of neural tissue loss and function by Positron Emission Tomography (PET) or functional Magnetic Resonance Imaging (MRI), blood tests, biopsies and electrical studies such as electromyographic data.

E. Method for Improving a Cognitive Function

AMPA receptors mediate transmission in brain networks responsible for a host of cognitive functions (e.g., see, U.S. Pat. No. 6,274,600). Additional applications contemplated for the compounds of the present invention include improving the performance of subjects with sensory-motor problems dependent upon brain networks utilizing AMPA receptors; improving the performance of subjects impaired in cognitive tasks dependent upon brain networks utilizing AMPA receptors; improving the performance of subjects with memory deficiencies; and the like.

Thus, in another aspect, the present invention provides methods for improving a cognitive function. In a preferred embodiment, this method comprises the steps of (a) administering to the mammal an amount of an AMPA-receptor allosteric upmodulator effective to increase the expression of the neurotrophic factor in the brain of the mammal; and (b) administering to the mammal an amount of a group 1 metabotropic glutamate receptor antagonist effective to increase the expression of the neurotrophic factor in the brain of the mammal above the level exhibited by step (a) alone; wherein the cognitive function in the mammal is improved.

In one embodiment, improving a cognitive function refers to effecting an at least about 10% improvement thereof. In other embodiments, improving a cognitive function refers to effecting an at least about 20%, an at least about 30%, an at least about 40%, an at least about 50%, an at least about 60%, an at least about 70%, an at least about 80%, an at least about 90% or an at least about 100% improvement thereof.

An improvement of a cognitive function is assessed, for example, by comparing the cognitive function before treatment to the cognitive function after treatment or by a standardized criterion.

In one embodiment, improving the cognitive function comprises decreasing the amount of time needed for a mammal to learn a cognitive, motor or perceptual task.

In one embodiment, the cognitive function is learning, for example, cognitive learning, affective learning, or psychomotor learning.

In another preferred embodiment, the cognitive function is intelligence, for example, linguistic intelligence, musical intelligence, spatial intelligence, bodily intelligence, interpersonal intelligence, intrapersonal intelligence, or logico-mathematical intelligence.

Alternatively, invention compounds, in suitable formulations, can be employed for increasing the time for which a mammal retains a cognitive, motor or perceptual task. As another alternative, invention compounds, in suitable formulations, can be employed for decreasing the quantity and/or severity of errors made in recalling a cognitive, motor or perceptual task. Such treatment may prove especially advantageous in individuals who have suffered injury to the nervous system, or who have endured disease of the nervous system, especially injury or disease which affects the number of AMPA receptors in the nervous system. Invention compounds are administered to the affected individual, and thereafter, the individual is presented with a cognitive, motor or perceptual task.

In another preferred embodiment of the present invention, an AMPAKINE® and a mGluR5 antagonist, as described herein, are used in a method of treating or ameliorating a decline in a cognitive function or a neurological function in a mammal. The decline of cognitive function can result from a neurological disorder, such as, a memory disorder (e.g., memory decline that can be associated with aging, Pick's Disease, Lewy Body Disease or a dementia associated, e.g., with Huntington's Disease or Alzheimer's Disease); a cognitive dysfunction (e.g., dyslexia, lack of attention, lack of alertness, lack of concentration, or lack of focus); an emotional disorder (e.g., manic, depression, stress, panic, anxiety, dysthemia, psychosis, a bipolar disorder); ataxia; Friedrich's ataxia; a movement disorder (e.g., tardive dyskinesia); a cerebro-vascular disease resulting from e.g., hypoxia; a behavioral syndrome or a neurological syndrome that may follow brain trauma, spinal cord injury or anoxia; a peripheral nervous system disorder; or a neuromuscular disorder. Memory can be spatial memory, working memory, reference memory, short-term memory, medium-term memory, or long-term memory.

Further examples of evidence of a therapeutic benefit include clinical evaluations of cognitive functions including, object identification, increased performance speed of defined tasks as compared to pretreatment performance speeds, and nerve conduction velocity studies.

F. Method for Treating a Neuropsychiatric Disorder

This invention also relates to treatment of psychiatic disorders by enhancement of receptor functioning in synapses in brain networks responsible for higher order behaviors. In particular, the invention provides methods for the use of AMPA receptor up-modulators and mGluR5 antagonists for the treatment of a neuropsychiatric disorder and/or syndrome, such as schizophrenia, depression, and anxiety.

1. Treatment of Schizophrenia

Schizophrenia is a chronic mental disease in which affected individuals show a range of symptoms, including positive (hallucinations, delusions, formal thought disorder), negative (social withdrawal, flattened affect) and cognitive (formal thought disorder, executive memory dysfunction) symptoms. The estimated prevalence of schizophrenia among humans is 0.2-2% worldwide.

Recently it was shown that in addition to a dopamine imbalance (Carlsson & Lindqvist, 1967, Acta Pharmacol Toxicol 20:140-144; Creese et al., 1976, Science 192:481-482), a reduced excitatory activity of the glutamatergic system could underlie some, if not many, symptoms displayed by the pathophysiology of a schizophrenic brain (Coyle, 1996, Harv Rev Psychiatry 3:241-253; Tamminga, 1998, Crit. Rev Neurobiol 12:21-36). In addition, abnormalities in a number of brain regions that are connected by glutamatergic circuits were found in schizophrenic brains (Andreasen et al., 1992, Arch Gen Psychiatry 49:943-958; Carpenter et al., 1993 Arch Gen Psychiatry 509:825-831; Weinberger and Berman, 1996, Philos Trans R Soc Lond B Biol Sci 351:1495-503). A possible beneficial effect of AMPAKINES® and antipsychiatric drugs in the treatment of schizophrenia has been reported by Johnson et al. (1999, J Pharmacol Exp Ther 289(1):392-7) and in U.S. Pat. No. 6,166,008. However, no effect on expression of neurotrophic factors, such as BDNF, was reported.

For the reasons set forth herein, drugs that enhance the functioning of AMPA receptors have significant benefits for the treatment of schizophrenia (see also, e.g., U.S. Pat. No. 5,773,434, incorporated by reference in its entirety). Such drugs should also ameliorate the cognitive symptoms that are not addressed by currently-used antipsychiatrics.

The present invention provides a method for the treatment of schizophrenia in a subject in need of such treatment. In a preferred embodiment, this method comprises the steps of: (a) administering to the mammal an amount of an AMPA-receptor allosteric upmodulator effective to increase the expression of the neurotrophic factor in the brain of the mammal; and (b) administering to the mammal an amount of a group 1 metabotropic glutamate receptor antagonist effective to increase the expression of the neurotrophic factor in the brain of the mammal above the level exhibited by step (a) alone; wherein the subject is treated. Administrating to the subject a therapeutically effective amount of an AMPAKINE® and a mGluR5 antagonist is effective to increase the expression of a neurotrophic factor in the brain of the subject, wherein the mGluR5 antagonist potentiates the effect of the AMPAKINE® on the expression of the neurotrophic factor, thereby treating the subject.

2. Treatment of Depression and Anxiety

Depression affects a large percentage of the general population and can produce devastating consequences to affected individuals, families, and society. Depression is generally characterized by the presence of major depressive episodes which are defined as being a period of at least two weeks during which, for most of the day and nearly every day, there is either depressed mood or the loss of interest or pleasure in all, or nearly all activities. The individual may also experience changes in appetite or weight, sleep and psychomotor activity; decreased energy; feelings of worthlessness or guilt; difficulty thinking, concentrating or making decisions; and recurrent thoughts of death or suicidal ideation, plans or attempts. One or more major depressive episodes may give rise to a diagnosis of major depressive disorder (Diagnostic and Statistical Manual of Mental Disorders, Fourth Edition, American Psychiatric Association, 1994).

Anxiety is an emotional condition characterized by feelings such as apprehension and fear accompanied by physical symptoms such as tachycardia, increased respiration, sweating and tremor. It is a normal emotion but when it is severe and disabling it becomes pathological.

Antidepressants, such as selective serotonin reuptake inhibitors (hereinafter referred to as SSRIs) and have become first choice therapeutics in the treatment of depression, certain forms of anxiety, and social phobias, because they are effective, well tolerated, and have a favorable safety profile compared to the classic tricyclic antidepressants. However, clinical studies on depression and anxiety disorders indicate that non-response to SSRIs is substantial, up to 30%. Further, antidepressants can induce or increase suicidal tendencies (Tsai et al., 2005, Med Hypotheses 65(5):942-6). Another, often neglected, factor in antidepressant treatment is compliance, which has a rather profound effect on the patient's motivation to continue pharmacotherapy.

Recently, some evidence linking BDNF to major depression disorder (MDD) and bipolar disorder (BD), and that BDNF exerts antidepressant activity in animal models of depression, has been reported (Hashimoto et al., 2004, Brain Res Brain Res Rev 45(2):104-14; Schumacher et al., 2005, Diol Psychiatry 58(4):307-14). For example, it has been reported that antidepressants increase central BDNF levels and activate the BDNF-tyrosine kinase receptor B (TrkB) pathway (Tsai et al., 2005, Med Hypotheses 65(5):942-6). Furthermore, clinical studies have demonstrated that serum levels of BDNF in drug-naïve patients with MDD are significantly decreased as compared with normal controls, and that BDNF might be an important agent for therapeutic recovery from MDD. Moreover, recent findings from family-based association studies have suggested that the BDNF gene is a potential risk locus for the development of BD (Hashimoto et al., 2004, Brain Res Brain Res Rev 45(2): 104-14).

Although the treatment of depression has been advanced by traditional antidepressant, improvements are needed. Accordingly, the present invention provides pharmaceutical compositions and methods for the treatment of depression and anxiety. Specifically, the compounds of the present invention (AMPAKINES® and mGluR5 antagonists) which have been shown to increase BDNF expression, provide novel therapeutic drugs for patients with mood disorders, such as depression and anxiety.

The present invention provides a method for the treatment of depression in a subject in need of such treatment. In a preferred embodiment, this method comprises the steps of: (a) administering to the mammal an amount of an AMPA-receptor allosteric upmodulator effective to increase the expression of the neurotrophic factor in the brain of the mammal; and (b) administering to the mammal an amount of a group 1 metabotropic glutamate receptor antagonist effective to increase the expression of the neurotrophic factor in the brain of the mammal above the level exhibited by step (a) alone; wherein the subject is treated. Administrating to the subject a therapeutically effective amount of an AMPAKINE® and a mGluR5 antagonist is effective to increase the expression of a neurotrophic factor in the brain of the subject, wherein the mGluR5 antagonist potentiates the effect of the AMPAKINE® on the expression of the neurotrophic factor, thereby treating the subject. Such subject is preferably a human, such as male or female human, child, adult or elderly.

G. Method For Treating Fragile X Syndrome

Fragile X syndrome is the most common form of inherited mental retardation worldwide, affecting 1 in 1500 men and 1 in 2500 women. The fragile X mental retardation syndrome is caused by unstable expansion of a CGG repeat in the fragile X mental retardation (FMR-1) gene and clinical expression is associated with a large expansion of the CGG repeat (de Vries et al., 1993, Eur J Hum Genet. 1(1):72-9). Most patients exhibit several neurological deficits, including moderate to severe mental retardation, seizures during childhood, visual spatial defects, learning difficulties, characteristics of autism and stress-related behaviors.

A Fragile X mouse model, fmr (tm1Cgr), with a disruption in the X-linked Fmr1 gene, shows three substantial deficits observed in several strains: (i) sensitivity to audiogenic seizures (AGS), (ii) tendency to spend significantly more time in the center of an open field, and (iii) enlarged testes (Yan et al., 2005, Neuropharmacology 49(7): 1053-66). Alterations in group 1 mGluR signaling were identified in the fmr1 (tm1Cgr) mouse. Subsequently, modulation of mGluR5 signaling by MPEP was shown to ameliorate some symptoms of the Fragile X Syndrome (Yan et al., 2005, Neuropharmacology 49(7):1053-66). Further stress-induced changes in BDNF and c-fos mRNA were reported to be altered in the cortical area in fragile X mutant mice supporting the hypothesis of a dysregulated hypothalamic-pituitary-adrenal axis in the fragile X syndrome (Ramirez et al., 2003, Soc Neurosci Abst 318.21; Lauterborn, 2004, Brain Res Mol Brain Res 131(1-2):101-9).

Thus, in another preferred aspect of the present invention, a method for the treatment of fragile X syndrome is provided. In one embodiment, this method comprises the steps of: (a) administering to the mammal an amount of an AMPA-receptor allosteric upmodulator effective to increase the expression of the neurotrophic factor in the brain of the mammal; and (b) administering to the mammal an amount of a group 1 metabotropic glutamate receptor antagonist effective to increase the expression of the neurotrophic factor in the brain of the mammal above the level exhibited by step (a) alone; wherein the subject is treated. Administrating to the subject a therapeutically effective amount of an AMPAKINE® and a mGluR5 antagonist effective to increase the expression of a neurotrophic factor in the brain of the subject, wherein the mGluR5 antagonist potentiates the effect of the AMPAKINE® on the expression of the neurotrophic factor, thereby treating the subject.

H. Method for Treating a Sexual Dysfunction

The present invention also provides methods, compositions, and kits for treating sexual dysfunction in mammalian subjects, particularly human males.

Male sexual dysfunction can be due to one or more causes, for example, male erectile disorder (associated with atherosclerosis of the arteries supplying blood to the penis; “arteriogenic” or “atherosclerotic” dysfunction); neurological sexual dysfunction (associated with neuropathy); psychological or “psychogenic” dysfunction (resulting, e.g., from anxiety or depression with no apparent substantial somatic or organic impairment); and erectile insufficiency (sometimes a side effect of certain drugs, such as beta-blockers) (see, e.g., U.S. Pat. No. 6,083,947, incorporated herein by reference in its entirety).

The present invention is based on the discovery that male sexual dysfunction can be treated with compounds that enhance the activity of AMPA receptors (see, U.S. Pat. No. 6,083,947). Thus, according to the present invention provides a method for treating a sexual dysfunction in a subject. The compounds of the present invention can also be used in a method of increasing sexual activity in males suffering from age-related sexual dysfunctions that may be treated with AMPAKINES® and mGluR5 antagonists. Further the compounds of the present invention can also be used in a method of diminishing the symptoms of sexual dysfunction.

In a preferred embodiment, these methods comprise the steps of: (a) administering to the mammal an amount of an AMPA-receptor allosteric upmodulator effective to increase the expression of the neurotrophic factor in the brain of the mammal; and (b) administering to the mammal an amount of a group 1 metabotropic glutamate receptor antagonist effective to increase the expression of the neurotrophic factor in the brain of the mammal above the level exhibited by step (a) alone; wherein the sexual dysfunction in the subject is treated or wherein the sexual activity in males is increased or wherein the symptoms of sexual dysfunction are diminished.

I. Method for Treating a Pathology Associated with Reduced Expression of Growth Hormone

Recently, age dependent dysfunction of hormonal systems has been postulated to be associated with the mammalian aging process (Crew et al., 1987, Endocrinology 121:1251-1255; Martinoli et al., 1991, Neuroendocrinology 57:607-615). For example, growth hormone (GH) blood levels in the elderly are lower than GH blood levels in younger populations, where lower GH blood levels have been theorized to be associated with symptoms of the aging process, such as decreases in lean body mass, muscle and bone. Current methods of treating diseases associated with endocrine system dysfunction involving the hyposecretion of one or more particular hormones have centered on direct hormonal replacement, e.g. synthetic or recombinant growth hormone for GH deficient youths. While such approaches can be successful, hormone replacement therapy can be associated with a number of different disadvantages, such as risk of pathogen transmission, delivery, over compensation of replacement hormone, and the like. As such, there continues to be an interest in the development of new methods of treating diseases characterized by endocrine system dysfunction.

Recently, the presence and distribution of AMPA-type glutamate receptors in the hypothalamus was reported (Aubry et al., 1996, Neurosci Lett 205(2):95-8; van den Pol et al., 1994, J Comp Neurol 343(3):428-44) supporting the hypothesis that glutamate may directly influence neurons in the hypothalamus through AMPA receptors. Further, effects of AMPA receptor agonists on the excitation of hypothalamic neurons and on the release of neuropeptides has been documented (e.g., Lopez et al., 1992, Endocrinology 130(4):1986-92; Parker and Crowley, 1993, Endocrinology 133(6):2847-54; Nissen et al., 1995, J Physiol 484(Pt2):415-24; As described herein, this invention provides compounds useful for the stimulation of AMPA receptors. Stimulation of AMPA receptors is believed to lead to an increase in the circulatory level of neuropeptides secreted by the hypothalamus. These neuropeptides include oxytocin (OT), vasopressin (arginine vasopressin, AVP), growth hormone releasing hormone (GHRH), growth hormone release-inhibiting hormone (somatostatin), prolactin release inhibitory factor (dopamine), gonadotropin-releasing hormone (GnRH), corticotropin-releasing hormone (CRH), and thyrotropin-releasing hormone (TRH). Hormones released by the pituitary in response to hypothalamic neuropeptide influence include growth hormone (GH), prolactin (PRL), follicle-stimulating hormone (FSH), luteinizing hormone (LH), luteinizing hormone-releasing hormone (LHRH), adrenocorticotropic hormone (ACTH, corticotropin), and thyrotropin (thyroid stimulating hormone, TSH.

Although the use of AMPAKINES® for increasing the circulatory level of neuropeptides and growth hormone has been disclosed (U.S. Pat. No. 6,620,808), the co-administration of an AMPAKINE and a mGluR5 antagonist to further increase this circulatory level, has not been reported in the art. Thus, in one aspect of the present invention, a method for modulating a mammalian endocrine system is provided, and in particular, a method for increasing the circulatory level of a neuropeptide in a mammalian host is provided. In a preferred embodiment, this method comprises the steps of (a) administering to the mammal an amount of an AMPA-receptor allosteric upmodulator effective to increase the expression of the neurotrophic factor in the brain of the mammal; and (b) administering to the mammal an amount of a group 1 metabotropic glutamate receptor antagonist effective to increase the expression of the neurotrophic factor in the brain of the mammal above the level exhibited by step (a) alone; wherein the circulatory level of the neuropeptide in the mammalian is increased. Preferred neuropeptides are described above.

Of particular interest is use of the subject compounds to treat diseases associated with dysfunction of the hypothalamus-pituitary hormonal system, where the dysfunction of this particular system results in the hyposecretion of one or more pituitary hormones, where the pituitary hormones are usually under the regulatory control of a neuropeptide secreted by the hypothalamus, particularly a neuropeptide secreted in response to binding of glutamate to an AMPA receptor of the hypothalamus.

Of particular interest is use of the subject methods to upregulate the production of endogenous hormone by the pituitary, where disease has resulted in a down regulation of hormone production by down regulating the production of the requisite hypothalamic stimulatory hormone. Thus, in this class of diseases, by administering an AMPAKINE® and a mGluR5 antagonist comprising pharmaceutical compositions to the host, one upregulates the production of the stimulatory hypothalamic neuropeptide, which in turn upregulates the production of endogenous hormone (e.g., growth hormone) by the pituitary, thereby increasing the circulatory levels of the hormone in the host.

Accordingly, one class of diseases which may be treated using the compounds of the present invention are diseases associated with hyposecretion of growth hormone, resulting in abnormally low circulatory levels of growth hormone in the mammal, where the hyposecretion is not the result of substantially complete failure in the capability of the pituitary to produce growth hormone. The subject method then results in an elevated circulatory level of growth hormone in the mammal compared to the level prior to treatment.

In one embodiment of the present invention, the mammalian host suffers from a disease associated with an abnormally low circulatory level of a neuropeptide. The disease can be associated with an age related decrease in the circulatory level of the neuropeptide. Alternatively, the disease is associated with down regulation in endogenous hormonal production.

J. Additional Uses of the Compounds of the Present Invention

The methods of the invention facilitate the effects of positive AMPA receptor modulators on increasing neurotrophin (e.g., BDNF) expression, thus promoting even greater increases in BDNF expression than would be accomplished by just the positive AMPA receptor modulator. As demonstrated above, this is accomplished by co-administration of an mGluR5 antagonist and a positive AMPA receptor modulator. Thus, this invention is particularly useful as a therapeutic treatment where larger increases in BDNF induction are desired. In addition to the above describe methods, the co-administration of an AMPAKINE® and a mGluR5 antagonist might also be useful in other instances of impaired brain function that might occur with aging and brain damage including damage arising from an untoward events such as stroke, heart attack, a period of anoxia or those that might occur with open heart surgery and other medical procedures.

### IV. Pharmaceutical Compositions

In one aspect the present invention provides a pharmaceutical composition or a medicament comprising at least an AMPAKINE® and a mGluR5 antagonist of the present invention and optionally a pharmaceutically acceptable carrier. A pharmaceutical composition or medicament can be administered to a subject for the treatment of, for example, a condition or disease as described herein.

A. Formulation and Administration

Compounds of the present invention, such as the AMPAKINES® and mGluR5 antagonists described herein, are useful in the manufacture of a pharmaceutical composition or a medicament comprising an effective amount thereof in conjunction or mixture with excipients or carriers suitable for either enteral or parenteral application.

Pharmaceutical compositions or medicaments for use in the present invention can be formulated by standard techniques using one or more physiologically acceptable carriers or excipients. Suitable pharmaceutical carriers are described herein and in “Remington's Pharmaceutical Sciences” by E. W. Martin. The small molecule compounds of the present invention and their physiologically acceptable salts and solvates can be formulated for administration by any suitable route, including via inhalation, topically, nasally, orally, parenterally, or rectally. Thus, the administration of the pharmaceutical composition may be made by intradermal, subdermal, intravenous, intramuscular, intranasal, intracerebral, intratracheal, intraarterial, intraperitoneal, intravesical, intrapleural, intracoronary or intratumoral injection, with a syringe or other devices. Transdermal administration is also contemplated, as are inhalation or aerosol administration. Tablets and capsules can be administered orally, rectally or vaginally.

For oral administration, a pharmaceutical composition or a medicament can take the form of, for example, a tablet or a capsule prepared by conventional means with a pharmaceutically acceptable excipient. Preferred are tablets and gelatin capsules comprising the active ingredient, i.e., a small molecule compound of the present invention, together with (a) diluents or fillers, e.g., lactose, dextrose, sucrose, mannitol, sorbitol, cellulose (e.g., ethyl cellulose, microcrystalline cellulose), glycine, pectin, polyacrylates and/or calcium hydrogen phosphate, calcium sulfate; (b) lubricants, e.g., silica, talcum, stearic acid, its magnesium or calcium salt, metallic stearates, colloidal silicon dioxide, hydrogenated vegetable oil, corn starch, sodium benzoate, sodium acetate and/or polyethyleneglycol; for tablets also (c) binders, e.g., magnesium aluminum silicate, starch paste, gelatin, tragacanth, methylcellulose, sodium carboxymethylcellulose, polyvinylpyrrolidone and/or hydroxypropyl methylcellulose; if desired (d) disintegrants, e.g., starches (e.g., potato starch or sodium starch), glycolate, agar, alginic acid or its sodium salt, or effervescent mixtures; (e) wetting agents, e.g., sodium lauryl sulphate, and/or (f) absorbents, colorants, flavors and sweeteners.

Tablets may be either film coated or enteric coated according to methods known in the art. Liquid preparations for oral administration can take the form of, for example, solutions, syrups, or suspensions, or they can be presented as a dry product for constitution with water or other suitable vehicle before use. Such liquid preparations can be prepared by conventional means with pharmaceutically acceptable additives, for example, suspending agents, for example, sorbitol syrup, cellulose derivatives, or hydrogenated edible fats; emulsifying agents, for example, lecithin or acacia; non-aqueous vehicles, for example, almond oil, oily esters, ethyl alcohol, or fractionated vegetable oils; and preservatives, for example, methyl or propyl-p-hydroxybenzoates or sorbic acid. The preparations can also contain buffer salts, flavoring, coloring, and/or sweetening agents as appropriate. If desired, preparations for oral administration can be suitably formulated to give controlled release of the active compound.

Compounds of the present invention can be formulated for parenteral administration by injection, for example by bolus injection or continuous infusion. Formulations for injection can be presented in unit dosage form, for example, in ampoules or in multi-dose containers, with an added preservative. Injectable compositions are preferably aqueous isotonic solutions or suspensions, and suppositories are preferably prepared from fatty emulsions or suspensions. The compositions may be sterilized and/or contain adjuvants, such as preserving, stabilizing, wetting or emulsifying agents, solution promoters, salts for regulating the osmotic pressure and/or buffers. Alternatively, the active ingredient can be in powder form for constitution with a suitable vehicle, for example, sterile pyrogen-free water, before use. In addition, they may also contain other therapeutically valuable substances. The compositions are prepared according to conventional mixing, granulating or coating methods, respectively, and contain about 0.1 to 75%, preferably about 1 to 50%, of the active ingredient.

For administration by inhalation, the compounds may be conveniently delivered in the form of an aerosol spray presentation from pressurized packs or a nebulizer, with the use of a suitable propellant, for example, dichlorodifluoromethane, trichlorofluoromethane, dichlorotetrafluoroethane, carbon dioxide, or other suitable gas. In the case of a pressurized aerosol, the dosage unit can be determined by providing a valve to deliver a metered amount. Capsules and cartridges of, for example, gelatin for use in an inhaler or insufflator can be formulated containing a powder mix of the compound and a suitable powder base, for example, lactose or starch.

Suitable formulations for transdermal application include an effective amount of a compound of the present invention with carrier. Preferred carriers include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin. Matrix transdermal formulations may also be used.

Suitable formulations for topical application, e.g., to the skin and eyes, are preferably aqueous solutions, ointments, creams or gels well-known in the art. Such may contain solubilizers, stabilizers, tonicity enhancing agents, buffers and preservatives.

The compounds can also be formulated in rectal compositions, for example, suppositories or retention enemas, for example, containing conventional suppository bases, for example, cocoa butter or other glycerides.

Furthermore, the compounds can be formulated as a depot preparation. Such long-acting formulations can be administered by implantation (for example, subcutaneously or intramuscularly) or by intramuscular injection. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

The compositions can, if desired, be presented in a pack or dispenser device that can contain one or more unit dosage forms containing the active ingredient. The pack can, for example, comprise metal or plastic foil, for example, a blister pack. The pack or dispenser device can be accompanied by instructions for administration.

In one embodiment of the present invention, a pharmaceutical composition or medicament comprises an effective amount of an AMPAKINE® and a mGluR5 antagonist of the present invention as defined above, and another therapeutic agent, such as an antidepressant, anti-psychotic, anti-epileptric, acetyl cholinesterase inhibitor, phosphodiesterase inhibitor (e.g., Type5), and adenosine A2A receptor inhibitors. When used with compounds of the invention, such therapeutic agent may be used individually (e.g., an antidepressant and compounds of the present invention), sequentially (e.g., an antidepressant and compounds of the present invention for a period of time followed by e.g., a second therapeutic agent and compounds of the present invention), or in combination with one or more other such therapeutic agents (e.g., an antidepressant, a second therapeutic agent, and compounds of the present invention). Administration may be by the same or different route of administration or together in the same pharmaceutical formulation.

B. Therapeutically Effective Amount and Dosing

In one embodiment of the present invention, a pharmaceutical composition or medicament is administered to a subject, preferably a human, at a therapeutically effective dose to prevent, treat, or control a condition or disease as described herein. The pharmaceutical composition or medicament is administered to a subject in an amount sufficient to elicit an effective therapeutic response in the subject. An effective therapeutic response is a response that at least partially arrests or slows the symptoms or complications of the condition or disease. An amount adequate to accomplish this is defined as “therapeutically effective dose.”

The dosage of active compounds administered is dependent on the species of warm-blooded animal (mammal), the body weight, age, individual condition, surface area or volume of the area to be treated and on the form of administration. The size of the dose also will be determined by the existence, nature, and extent of any adverse effects that accompany the administration of a particular small molecule compound in a particular subject. A unit dosage for oral administration to a mammal of about 50 to 70 kg may contain between about 5 and 500 mg of the active ingredient. Typically, a dosage of the active compounds of the present invention, is a dosage that is sufficient to achieve the desired effect. Optimal dosing schedules can be calculated from measurements of compound accumulation in the body of a subject. In general, dosage may be given once or more daily, weekly, or monthly. Persons of ordinary skill in the art can easily determine optimum dosages, dosing methodologies and repetition rates.

In one embodiment of the present invention, a pharmaceutical composition or medicament comprising compounds of the present invention is administered in a daily dose in the range from about 1 mg of each compound per kg of subject weight (1 mg/kg) to about 1 g/kg for multiple days. In another embodiment, the daily dose is a dose in the range of about 5 mg/kg to about 500 mg/kg. In yet another embodiment, the daily dose is about 10 mg/kg to about 250 mg/kg. In another embodiment, the daily dose is about 25 mg/kg to about 150 mg/kg. A preferred dose is about 10 mg/kg. The daily dose can be administered once per day or divided into subdoses and administered in multiple doses, e.g., twice, three times, or four times per day. However, as will be appreciated by a skilled artisan, AMPAKINES® and mGluR5 antagonists may be administered in different amounts and at different times.

To achieve the desired therapeutic effect, compounds may be administered for multiple days at the therapeutically effective daily dose. Thus, therapeutically-effective administration of compounds to treat a condition or disease described herein in a subject requires periodic (e.g., daily) administration that continues for a period ranging from three days to two weeks or longer. Typically, compounds will be administered for at least three consecutive days, often for at least five consecutive days, more often for at least ten, and sometimes for 20, 30, 40 or more consecutive days. While consecutive daily doses are a preferred route to achieve a therapeutically effective dose, a therapeutically beneficial effect can be achieved even if the compounds are not administered daily, so long as the administration is repeated frequently enough to maintain a therapeutically effective concentration of the compounds in the subject. For example, one can administer the compounds every other day, every third day, or, if higher dose ranges are employed and tolerated by the subject, once a week.

In a preferred treatment regimen, a therapeutically effective concentration of BDNF is maintained while treating a subject.

Optimum dosages, toxicity, and therapeutic efficacy of such compounds may vary depending on the relative potency of individual compounds and can be determined by standard pharmaceutical procedures in cell cultures or experimental animals, for example, by determining the LD₅₀ (the dose lethal to 50% of the population) and the ED₅₀ (the dose therapeutically effective in 50% of the population). The dose ratio between toxic and therapeutic effects is the therapeutic index and can be expressed as the ratio, LD₅₀/ED₅₀. Compounds that exhibit large therapeutic indices are preferred. While compounds that exhibit toxic side effects can be used, care should be taken to design a delivery system that targets such compounds to the site of affected tissue to minimize potential damage to normal cells and, thereby, reduce side effects.

The data obtained from, for example, cell culture assays and animal studies can be used to formulate a dosage range for use in humans. The dosage of such small molecule compounds lies preferably within a range of circulating concentrations that include the ED₅₀ with little or no toxicity. The dosage can vary within this range depending upon the dosage form employed and the route of administration. For any compounds used in the methods of the invention, the therapeutically effective dose can be estimated initially from cell culture assays. A dose can be formulated in animal models to achieve a circulating plasma concentration range that includes the IC₅₀ (the concentration of the test compound that achieves a half-maximal inhibition of symptoms) as determined in cell culture. Such information can be used to more accurately determine useful doses in humans. Levels in plasma can be measured, for example, by high performance liquid chromatography (HPLC). In general, the dose equivalent of compounds is from about 1 ng/kg to 100 mg/kg for a typical subject.

Following successful treatment, it may be desirable to have the subject undergo maintenance therapy to prevent the recurrence of the condition or disease treated.

### V. Kits

For use in diagnostic, research, and therapeutic applications suggested above, kits are also provided by the invention. In the diagnostic and research applications such kits may include any or all of the following: assay reagents, buffers, a compounds of the present invention, a neurotrophic factor polypeptide, a neurotrophic factor nucleic acid, an anti-neurotrophic factor antibody, hybridization probes and/or primers, neurotrophic factor expression constructs, etc. A therapeutic product may include sterile saline or another pharmaceutically acceptable emulsion and suspension base.

In a preferred embodiment of the present invention, a kit comprises one or more AMPA-receptor allosteric upmodulator (e.g., an AMPAKINE®) and one or more mGluR5 antagonists.

In addition, a kit may include instructional materials containing directions (i.e., protocols) for the practice of the methods of this invention. The instructions may be present in the subject kits in a variety of forms, one or more of which may be present in the kit. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

In a preferred embodiment of the present invention, the kit comprises an instruction for using an AMPA-receptor allosteric upmodulator and a group 1 metabotropic glutamate receptor 5 antagonist for increasing the level of a neurotrophic factor above the level of neurotrophic factor induced by the AMPA-receptor allosteric upmodulator alone.

Optionally, the instruction comprises warnings of possible side effects and drug-drug or drug-food interactions.

A wide variety of kits and components can be prepared according to the present invention, depending upon the intended user of the kit and the particular needs of the user.

In a preferred embodiment of the present invention, the kit is a pharmaceutical kit and comprises a pharmaceutical composition comprising (i) an AMPAKINE®, (ii), a mGluR5 antagonist, and (iii) a pharmaceutical acceptable carrier. Pharmaceutical kits optionally comprise an instruction stating that the pharmaceutical composition can or should be used for treating a condition or disease described herein.

Additional kit embodiments of the present invention include optional functional components that would allow one of ordinary skill in the art to perform any of the method variations described herein.

Although the forgoing invention has been described in some detail by way of illustration and example for clarity and understanding, it will be readily apparent to one of ordinary skill in the art in light of the teachings of this invention that certain variations, changes, modifications and substitution of equivalents may be made thereto without necessarily departing from the spirit and scope of this invention. As a result, the embodiments described herein are subject to various modifications, changes and the like, with the scope of this invention being determined solely by reference to the claims appended hereto. Those of skill in the art will readily recognize a variety of non-critical parameters that could be changed, altered or modified to yield essentially similar results.

While each of the elements of the present invention is described herein as containing multiple embodiments, it should be understood that, unless indicated otherwise, each of the embodiments of a given element of the present invention is capable of being used with each of the embodiments of the other elements of the present invention and each such use is intended to form a distinct embodiment of the present invention.

The referenced patents, patent applications, and scientific literature, including accession numbers to GenBank database sequences, referred to herein are hereby incorporated by reference in their entirety as if each individual publication, patent or patent application were specifically and individually indicated to be incorporated by reference. Any conflict between any reference cited herein and the specific teachings of this specification shall be resolved in favor of the latter. Likewise, any conflict between an art-understood definition of a word or phrase and a definition of the word or phrase as specifically taught in this specification shall be resolved in favor of the latter.

As can be appreciated from the disclosure above, the present invention has a wide variety of applications. The invention is further illustrated by the following examples, which are only illustrative and are not intended to limit the definition and scope of the invention in any way.

### VI. EXAMPLES

### Example 1

### General Methods

1. Tissue Samples

Cultured hippocampal slices were prepared from rat pups (9 d postnatal) essentially as described by Lauterborn et al. (Lauterborn et al., 2000, J Neurosci 20(1):8-21). Slices were explanted onto Millicel-CM biomembrane inserts (Millipore, Bedford, Mass.; 6 slices/membrane) in a 6-well culture cluster plate (Corning, Cambridge, Mass.) containing sterile media (1 ml/well) consisting of minimum essential media, 30 mM dextrose, 30 mM HEPES, 5 mM Na₂HCO₃, 3 mM glutamine, 0.5 mM ascorbic acid, 2 mM CaCl₂, 2.5 mM MgSO₄, 1 mg/l insulin and 20% horse serum (pH 7.2; all reagents from Sigma, St. Louis, Mo.) and maintained for 10-18 d in a humidified incubator at 37° C. in 5% CO₂. Media was changed three times/week.

2. Treatment with AMPAKINES® and mGluR5 Antagonists

All experiments with the AMPAKINE® (Cortex Pharmaceuticals) and mGluR5 antagonist (gift from FRAXA Research Foundation) began on days 11-12 in culture and were performed essentially as described by Lauterborn et al. (Lauterborn et al., 2000, J Neurosci 20(1):8-21) and Huber et al. (Huber et al., 2002, Proc Natl Acad Sci USA 99(11):7746-50). AMPAKINES® were dissolved in 100% dimethylsulfoxide (DMSO; Sigma) and stored at −20° C. MPEP was dissolved in 100% DMSO. Briefly, CX614 (LiD37 or BDP-37) (Arai et al., 1997, Soc Neurosci Abstr 23:313; Hennegrif et al., 1997, J Neurchem 68:2424-2434; Kessler et al., 1998, Brain Res 783:121-126) was used at either 20 or 50 μM, and MPEP was used at 50 μM. For controls, cultures were either untreated or treated with equivalent concentrations of vehicle (i.e., DMSO at final concentrations of 1:2,000-1:10,000). The control experiments demonstrated that treatment with DMSO vehicle alone had no significant effect on BDNF mRNA expression.

3. cRNA Probe Preparation and In Situ Hybridization

cRNA probes were transcribed in the presence of ³⁵S-labeled UTP (DuPont NEN, Boston, Mass.). The cRNA to BDNF exon V was generated from PvuII-digested recombinant plasmid pR1112-8 (Isackson et al., 1991, Neuron 6:937-948), yielding a 540 base length probe with 384 bases complementary to BDNF exon V-containing mRNA (Timmusk et al., 1993, Neuron 10:475-489).

In situ hybridization was performed essentially as described by Lauterborn et al. (Lauterborn et al., 2000, J Neurosci 20(1):8-21; Lauterborn et al., 1994, Mol Cell Neurosci 5:46-62). Briefly, for in situ hybridization analyses, treatments were terminated by slice fixation with 4% paraformaldehyde in 0.1 M phosphate buffer, pH 7.2 (PPB). Cultures were re-sectioned parallel to the broad explant surface, slide-mounted, and processed for the in situ hybridization localization of BDNF mRNA using the ³⁵S-labeled BDNF cRNA probe described above. Following hybridization, the tissue was processed for film (Kodak Biomax) autoradiography.

Quantification of in situ hybridization was performed essentially as described by Lauterborn et al. (Lauterborn et al., 2000, J Neurosci 20(1):8-21). Briefly, for quantification of in situ hybridization, hybridization densities were measured from film autoradiograms, with labeling densities calibrated relative to film images of ¹⁴C-labeled standards (μCi/g), using the AIS system (Imaging Research Inc.). Significance was determined using the two-way ANOVA followed by Student-Newman-Keuls (SNK) or Student's t tests for individual comparisons.

4. BDNF Immunoassay

BDNF immunoassay was performed essentially as described by Lauterborn et al. (Lauterborn et al., 2000, J Neurosci 20(1):8-21). Cultures were collected into 100 μl of cold lysis buffer (137 mM NaCl, 20 mM Tris, 10% glycerol, 1 mM PMSF, 10 μg/ml aprotinin, 1 μg/ml leupeptin, 0.5 mM Na vanadate, and 1% NP-40). Four hippocampal slices from one insert were pooled for each “sample” assayed; each time point included three to four separate samples. Tissue was manually homogenized in lysis buffer, acidified to pH 2.5 with 1N HCl, and incubated for 15 min on ice. The pH was neutralized to pH 8.0 with 1N NaOH, and samples were frozen (−70° C.) until assayed. Total BDNF protein content for each sample was measured using the BDNF Emax Immunassay System (Promega, Madison, Wis.) according to kit instructions, with the absorbance at 450 nm determined using a plate reader. Data from two separate immunoassay experiments were pooled for statistical analyses using ANOVA followed by the Student-Newman-Keuls test for individual comparisons.

5. Western Blotting

For protein determinations, drug-treated and vehicle-treated hippocampal slice cultures were homogenized in RIPA (Radio-Immunoprecipitation Assay) buffer containing 10 mM Tris, pH 7.2, 158 mM NaCl, 1 mM EDTA, 0.1% SDS, 1% sodium deoxycholate, 1% triton-X, Complete Protease Inhibitor Cocktail (Roche Diagnostics; Indianapolis, Ind.), and Phosphatase Inhibitor Cocktails 1 and 2 (P2850 and 5726, Sigma), normalized for protein content using the Bio-Rad protein assay, and analyzed by Western blot analysis. Following addition of reducing SDS-polyacrylamide gel electrophoresis sample buffer, protein samples were separated on 4-20% gradient gels, transferred to polyvinylidene difluoride membranes, and incubated with antibodies specific for BDNF (1:2000, Santa Cruz Biotechnology). Binding of anti-BDNF antibodies to BDNF was detected by enhanced chemiluminescence. Band densities were quantified using ImageQuant software (Molecular Dynamics, Sunnyvale, Calif.).

### Example 2

### AMPAKINES® Increase Hippocampal BDNF mRNA Expression In Vitro: Supra-Threshold CX614 Dose Elevates Levels Through 24 h

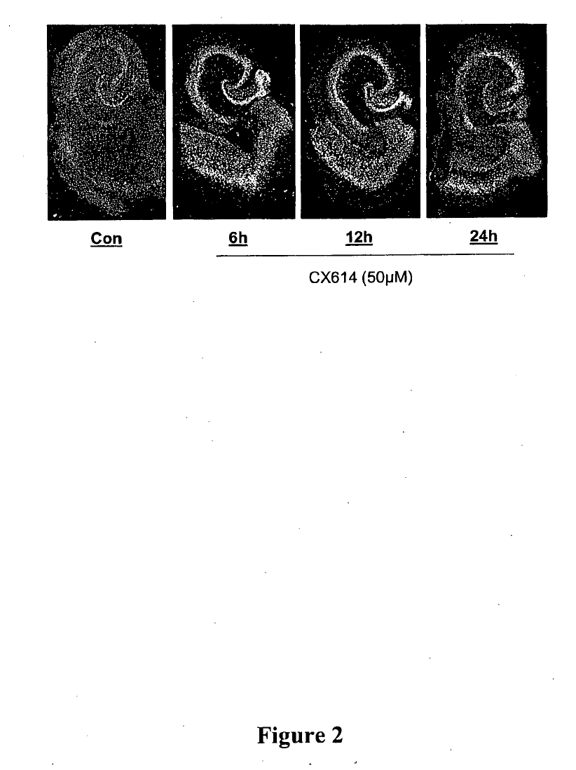

Cultured rat hippocampal slices were treated for 6 h, 12 h or 24 h with the positive AMPA receptor modulator CX614 (50 μM). Control (vehicle-treated) and CX614-treated cultures were processed for the in situ hybridization localization of BDNF mRNA. Photomicrographs (dark-field) show BDNF cRNA labeling (FIG. 2). Hybridization to BDNF mRNA was increased by CX614 treatment throughout the principal hippocampal cell layers, entorhinal cortex, and neocortex by 6 h. With 24 h treatment, levels were beginning to decline although they were still elevated above control densities.

### Example 3

### Treatment with mGluR5 Antagonist MPEP Potentiates CX614-Induced Increases in Hippocampal BDNF mRNA

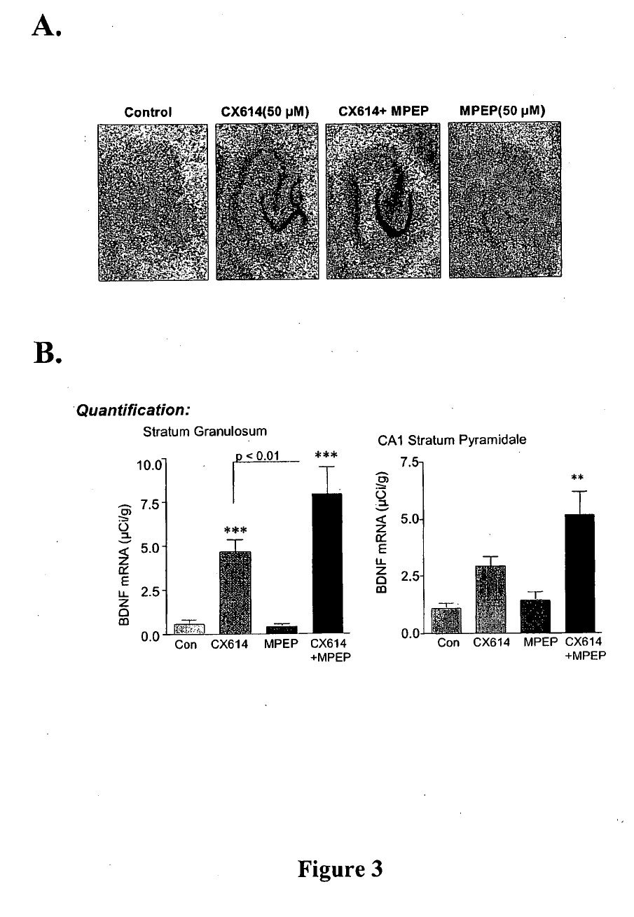

Cultured rat hippocampal slices were treated for 3 h with the positive AMPA receptor modulator CX614 (50 μM) with and without the mGluR5 antagonist MPEP (50 μM) as described herein. In situ hybridization analysis of BDNF mRNA in the hippocampal granule cells revealed a 6.5-fold increase in BDNF mRNA in cultures treated with CX614 alone (p<0.001 vs control group). In cultures co-treated with CX614+MPEP, BDNF mRNA levels were increased 10.5-fold above control levels (p<0.001) and were significantly greater than levels in the CX614 alone group (p<0.01). In cultures treated with MPEP alone BDNF mRNA levels in the granule cell layer were unaffected. Similar effects were seen in the pyramidal cell layer of hippocampal region CA1, where CX614+MPEP lead to greater increases (p<0.01) in BDNF mRNA levels than CX614 alone. A representative result is shown in FIG. 3.

### Example 4

### Effect of CX614 on BDNF Expression is Dose-Dependent

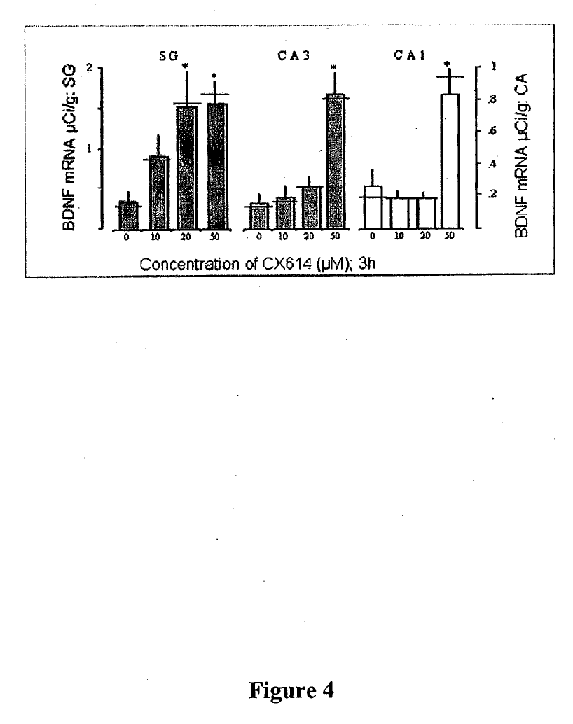

Cultures were treated with various concentrations of CX614 (10, 20, or 50 μM) for 3 h. In situ hybridization analysis of BDNF mRNA in the hippocampal granule cells and pyramidal cell layers of regions CA1 and CA3 revealed differences in the dose-response between these fields. Whereas BDNF mRNA was only increased with the 50 μM concentration of CX614 in the pyramidal cells (p<0.05 versus control group), it was increased by all three doses in the granule cells; increases above control levels were significant at the two highest concentrations (p<0.05). A representative result is shown in FIG. 4.

### Example 5

### Treatment with a Low Dose of CX614 is Potentiated by mGluR5 Antagonist

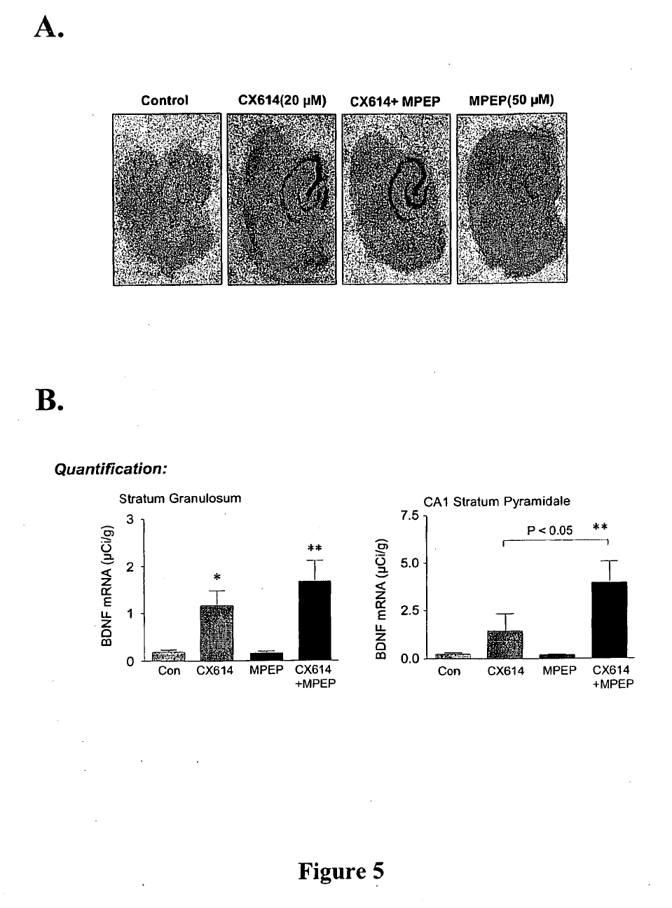

Cultures were treated with CX614 (20 μM) for 24 h with and without MPEP co-application as described herein. For the granule cell layer, there were slightly greater BDNF mRNA levels in the CX614+MPEP group than the CX614 alone group. In the CA1 pyramidal cells, treatment with the low dose of CX614 alone for 24 h lead to a small but non-significant increase in BDNF mRNA levels. However, in cultures co-treated for 24 h with CX614+MPEP, BDNF mRNA levels in CA1 were markedly increased above control levels (p<0.01) and above the CX614 alone group (p<0.05). These data demonstrate that the mGluR5 antagonist MPEP enhances the effective dose of a positive AMPA receptor modulator on BDNF expression within hippocampus. A representative result is shown in FIG. 5.

### Example 6

### Treatment with MPEP Attenuates CX614-Induced Decline in GluR Expression

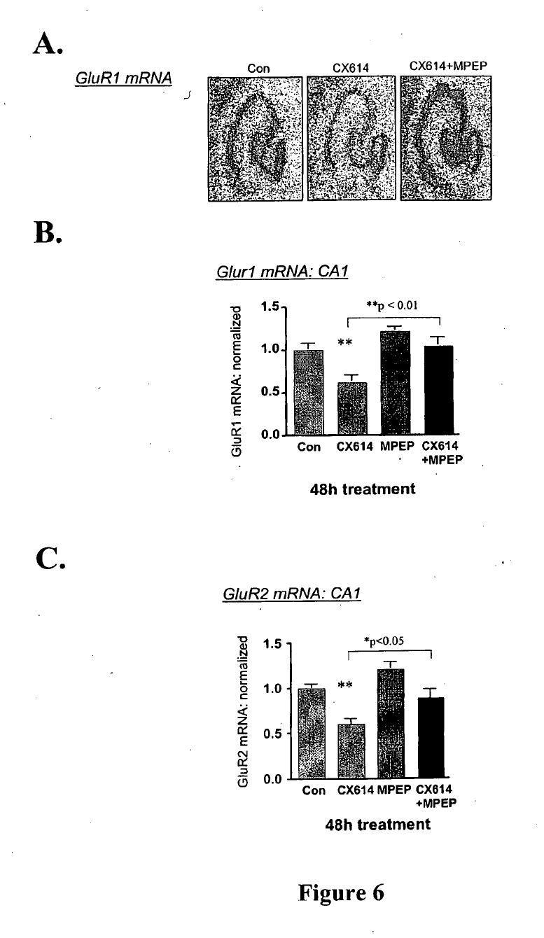

Cultures were treated with CX614 (50 μM) for 48 h with and without MPEP co-application as described herein. In situ hybridization analysis revealed that treatment with CX614 alone reduced hippocampal region CA1 pyramidal cell layer GluR1 and GluR2 mRNA levels (p<0.01) by 40-45% as compared to control levels. However, in cultures co-treated for 48 h with CX614+MPEP, the decrease in GluR1 and GluR2 mRNA levels was attenuated (i.e., GluR2 mRNA; p<0.05) or blocked (i.e., GluR1 mRNA; p<0.01). Treatment with MPEP alone had no significant effect on GluR mRNA levels. A representative result is shown in FIG. 6. An alternative method to maintain appropriate GluR levels would be to use an AMPAKINE® regimen of “pulsing” for short periods of time followed by drug removal (or metabolization).

Thus, administration of MPEP may show two benefits: (i) in the short-term it potentiates BDNF levels via effects on AMPA receptor surface expression or on calcium-mediated processes and (ii) in the long-term it potentiates BDNF levels by maintaining GluR levels (i.e., blocking AMPAKINE®-induced decreases in GluR mRNA); thus, allowing for the AMPAKINE® to have effects for a longer period of time.

### Example 7

### MPEP Co-Administration Increases CX614-Induced Mature BDNF Protein Levels in Organotypic Hippocampal Cultures

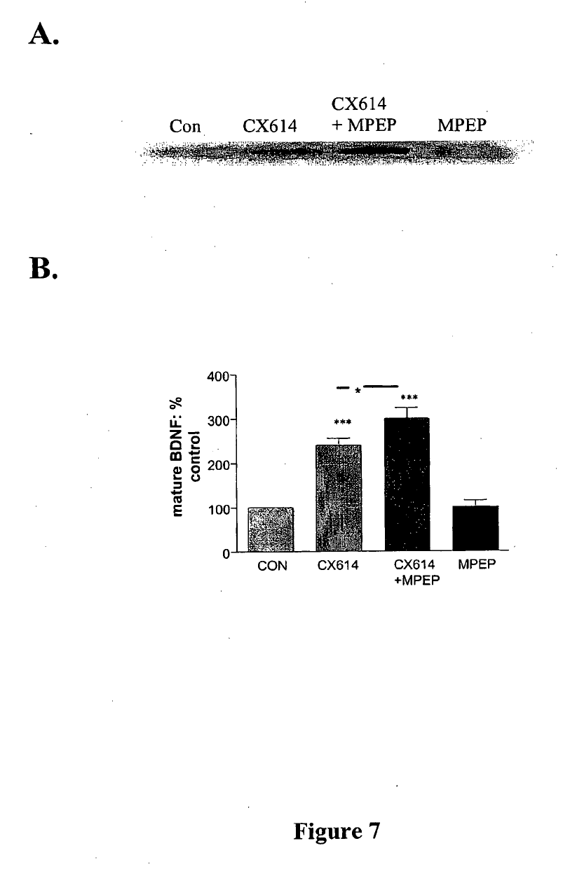

Cultures were treated with CX614 (50 μM) for 24 h with and without MPEP co-application as described herein. Four hippocampal cultures were pooled for each sample assayed. Western blot analysis for mature BDNF protein levels in the cultures revealed that CX614 increased BDNF protein levels to 133% above control-levels (p<0.001). Co-administration of MPEP+CX614 lead to a greater (25%) increase in total mature BDNF levels than CX614 alone (p<0.05 for MPEP+CX614 versus CX614 alone groups). A representative result is shown in FIG. 7.

### Example 8

### In Vivo CX929 Treatment Increases BDNF Protein Levels in Hippocampus

Adult male rats were injected intraperitoneally twice per day, 6 h apart, for 4 days with CX929 (1, 2.5, and 5 mg/kg). Immediately after AMPAKINE® or vehicle injections, animals, were placed, as groups, in an enriched environment consisting of a wedge-shaped box with partitions and platforms for exploration and social interaction. Eighteen hours after the last injection, animals were killed and hippocampal samples were collected and processed for BDNF ELISA as described herein. In rats receiving CX929 injections, BDNF protein levels were significantly increased by all three doses, with the 1 mg/kg and 2.5 mg/kg doses resulting in nearly the same increase in BDNF protein levels to 55-65% above control levels (p<0.05). The highest dose (5 mg/kg) showed the greatest effect as compared to control levels with increases at 125% above control levels (p<0.001). A representative result is shown in FIG. 8.

What is claimed is:

1. A method for increasing the level of a neurotrophic factor in a brain of a mammal afflicted with a neurodegenerative pathology, the method comprising the steps of:
(a) administering to the mammal an amount of an AMPA-receptor allosteric upmodulator effective to increase the expression of the neurotrophic factor in the brain of the mammal; and
(b) administering to the mammal an amount of a group 1 metabotropic glutamate receptor antagonist effective to increase the expression of the neurotrophic factor in the brain of the mammal above the level exhibited by step (a) alone.

12. The method according to claim 1, wherein administering the group 1 metabotropic glutamate receptor antagonist increases the level of the neurotrophic factor at least 25% above the level exhibited by step (a) alone.

13. The method according to claim 1, wherein the neurodegenerative pathology is selected from the group consisting of Parkinson's Disease, amyotrophic lateral sclerosis (ALS), Huntington's disease, and Down's Syndrome.

4. The method according to claim 1, wherein the neurodegenerative pathology is characterized by reduced cognitive activity.

5. The method according to claim 1, wherein the neurodegenerative pathology is a psychiatric disorder.

6. The method according to claim 1, wherein the neurodegenerative pathology is Fragile X syndrome.

7. The method according to claim 1, wherein the neurodegenerative pathology is a sexual dysfunction.

8. The method according to claim 1, wherein the neurodegenerative pathology is characterized by reduced expression of a growth hormone.

9. The method according to claim 1, wherein the mammal is a human.

10. The method according to claim 1, wherein the neurotrophic factor is selected from the group consisting of brain derived neurotrophic factor, nerve growth factor, glial cell line derived neurotrophic factor, ciliary neurotrophic factor, fibroblast growth factor, and insulin-like growth factor.

11. The method according to claim 10, wherein the neurotrophic factor is brain derived neurotrophic factor.

12. The method according to claim 1, wherein the AMPA-receptor allosteric upmodulator is blood-brain barrier permeant.

13. The method according to claim 1, wherein the group 1 metabotropic glutamate receptor antagonist is blood-brain barrier permeant.

14. The method according to claim 1, wherein the group 1 metabotropic glutamate receptor antagonist is selected from the group consisting of 2-methyl-6-(phenylethynyl)pyridine (MPEP), 3-[(2-methyl-1,3-thiazol-4-yl)ethynyl]pyridine (MTEP), (E)-2-methyl-6-styryl-pyridine (SIB 1893), N-(3-chlorophenyl)-N′-(4,5-dihyfro-1-methyl-4-oxo-1H-imidazole-2-yl)urea (fenobam), and structural analogs thereof.

15. The method according to claim 14, wherein the group 1 metabotropic glutamate receptor antagonist is MPEP.

16. The method according to claim 14, wherein the group 1 metabotropic glutamate receptor antagonist is fenobam.

17. The method according to claim 1, wherein the AMPA-receptor allosteric upmodulator is selected from the group consisting of CX516, CX546, CX614, CX691, CX717, CX929, and structural analogs thereof.

18. The method according to claim 17, wherein the AMPA-receptor allosteric upmodulator is CX614.

19. The method according to claim 1, wherein the AMPA-receptor allosteric upmodulator is selected from the group consisting of 1, compound 2, compound 3, compound 4, compound 5, compound 6, compound 7, compound 8, compound 9, compound 10, compound 11, compound 12, compound 13, compound 14, compound 15, compound 16, compound 17, compound 18, compound 19, compound 20, compound 21, compound 22, compound 23, compound 24, compound 25, compound 26, compound 27, compound 28, compound 29, compound 30, compound 31, compound 32, compound 33, compound 34, compound 35 compound 36, compound 37, compound 38, compound 39, compound 40, compound 41, compound 42, compound 43, compound 44, compound 45 compound 46, compound 47, compound 48, compound 49, compound 50, compound 51, compound 52, compound 53, compound 54, and structural analogs thereof.

20. A method for increasing in a brain of a mammal afflicted with a neurodegenerative pathology the level of a neurotrophic factor above the level of neurotrophic factor induced by an AMPA-receptor allosteric upmodulator, the method comprising the step of:
(a) administering to the mammal an amount of a group 1 metabotropic glutamate receptor antagonist effective to increase the level of the neurotrophic factor in the brain of the mammal.

21. A pharmaceutical composition comprising:
(i) an AMPA-receptor allosteric upmodulator;
(ii) a group 1 metabotropic glutamate receptor antagonist; and
(iii) a pharmaceutically acceptable carrier.

22. Use of
(i) an AMPA-receptor allosteric upmodulator; and
(ii) a group 1 metabotropic glutamate receptor antagonist
in the manufacture of a medicament for increasing in a brain of a mammal afflicted with a neurodegenerative pathology the level of a neurotrophic factor.

23. A kit comprising:
(i) a first container containing an AMPA-receptor allosteric upmodulator;
(ii) a second container containing a group 1 metabotropic glutamate receptor 5 antagonist; and
(iii) an instruction for using the AMPA-receptor allosteric upmodulator and the group 1 metabotropic glutamate receptor 5 antagonist for increasing the level of a neurotrophic factor above the level of neurotrophic factor induced by the AMPA-receptor allosteric upmodulator alone.
